(19) United States
(12) Patent Application Publication
(10) Pub. No.: US 2026/0356967 A1
(43) Pub. Date: Nov. 12, 2026

(54) MULTISPECIFIC ANTIBODIES TO CD3 CD19 AND BCMA

(71) Applicant: Kali Therapeutics Inc., San Mateo, CA (US)

(72) Inventors: Zhong Wang, Foster City, CA (US); Weihao Xu, Belmont, CA (US)

(73) Assignee: Kali Therapeutics Inc., San Mateo, CA (US)

(21) Appl. No.: 19/659,638

(22) Filed: Apr. 27, 2026

Related U.S. Application Data
(60) Provisional application No. 63/796,249, filed on Apr. 28, 2025.

(51) Int. Cl. C07K 16/28; A61K 39/00; A61P 35/00; A61P 37/04

(52) U.S. Cl. CPC C07K 16/2878; A61P 35/00; A61P 37/04; C07K 16/2803; C07K 16/2809; A61K 2039/505; C07K 2317/31; C07K 2317/515; C07K 2317/522; C07K 2317/524; C07K 2317/526; C07K 2317/565; C07K 2317/569; C07K 2317/92

(57) ABSTRACT

Multispecific antibodies having specificity to CD3, CD19 and BCMA are provided. These multispecific antibodies have a significantly improved therapeutic index and are useful for treating cancers, autoimmune diseases and/or conditions with reduced toxicities.

### CROSS-REFERENCE TO RELATED APPLICATIONS

This application claims priority to U.S. Application No. 63/796,249, filed Apr. 28, 2025, the entire contents of which are incorporated by reference herein for all purposes.

### REFERENCE TO ELECTRONIC SEQUENCE LISTING

This application contains a Sequence Listing which has been submitted electronically in .XML format and is hereby incorporated by reference in its entirety. Said .XML copy, created on Apr. 14, 2026, is named “01183-0367-00PCT-ST26.xml” and is 81,664 bytes in size. The sequence listing contained in this .XML file is part of the specification and is hereby incorporated by reference herein in its entirety.

### BACKGROUND

Antibodies having binding specificity to CD3 and one or more tumor antigens are believed to have improved therapeutic efficacy as compared to conventional therapies. Such bispecific antibodies ensure direct connection of the T-cell to the cancer cell and thus can enable T-cell activation, which results in cytotoxic activity directed to the cancer cell.

Currently, there are many bispecific or trispecific antibodies with an anti-CD3 portion. These bispecific or trispecific antibodies come with different molecular designs and binding properties, and are tested in preclinical and clinical development for the treatment of liquid or solid tumors.

Although many of them showed potent cancer cell killing in vitro, their developments are often accompanied with safety concerns. These antibodies tend to cause excessive release of cytokines, which may lead to potentially life-threating cytokine release syndrome (CRS), target organ toxicity due to redirection of T-cells to normal tissues expressing the tumor-associated antigen (TAA) (off-tumor/on-target cytotoxicity), and, in some instances, neurotoxicity.

To expand the therapeutic index and bring more treatment options to patients, there is a need of CD3-based multispecific antibodies with reduced toxicity concerns.

### SUMMARY

The present disclosure provides both (1) a new format of multispecific antibodies and (2) combined antigen-binding polypeptides to three particular targets. As illustrated in antibody Ab101 (and Ab101-1) of FIG. 1, the new format includes a monovalent anti-CD3 portion that includes an anti-CD3 Fab fused to one of the two chains of an Fc fragment. Three single domain antibodies (VHH) are individually fused to the N-termini of the three peptide chains of the monovalent anti-CD3 portion. The presence of the two VHH on the N-terminal ends of the Fab, in particular, provides moderate inhibition to the anti-CD3 Fab. As needed, the distance of the VHH to the VH/VL of the Fab can be fine-tuned to achieve desired anti-CD3 activities. The three VHH, on the other hand, provide additional binding specificities to targets such as tumor antigens and immune cell markers.

With such reduced anti-CD3 activity, the multispecific antibodies have improved therapeutic index and can be more safely used for treating diseases such as cancer and autoimmune diseases. For autoimmune diseases, in particular, these multispecific antibodies can be used to deplete B cells without causing severe side effects such as cytokine release syndrome.

One aspect of the present disclosure provides a multispecific antibody comprising: (a) a first chain comprising an anti-BCMA single domain antibody (VHH), an anti-CD3 heavy chain variable region (VH), a CH1, a CH2, and a CH3; (b) a second chain comprising a first anti-CD19 VHH, an anti-CD3 light chain variable region (VL), and a light chain constant domain (CL); and (c) a third chain comprising a second anti-CD19 VHH, a CH2, and a CH3, wherein (i) the anti-BCMA VHH comprises a CDR1, CDR2, and CDR3 comprising, respectively, the amino acid sequences of SEQ ID NOs:8, 9 and 10; (ii) the first and second anti-CD19 VHH each independently comprising a CDR1, CDR2, and CDR3 comprising, respectively, the amino acid sequences of SEQ ID NOs:3, 4 and 5; (iii) the anti-CD3 VH comprises a H-CDR1, H-CDR2, and H-CDR3, the anti-CD3 VL comprises a L-CDR1, L-CDR2, and L-CDR3, and the H-CDR1, H-CDR2, H-CDR3, L-CDR1, L-CDR2, and L-CDR3 comprise, respectively, the amino acid sequences of SEQ ID NOs:16, 17, 18, 22, 23, and 24.

In some embodiments, the anti-BCMA VHH is N-terminal to the anti-CD3 VH, the first anti-CD19 VHH is N-terminal to the anti-CD3 VL, and the second anti-CD19 VHH is N-terminal to the CH2 of the third chain. In some embodiments, the anti-BCMA VHH comprises the amino acid sequence of SEQ ID NO:6 or 7. In some embodiments, the anti-BCMA VHH comprises an amino acid sequence having at least 90%, 91%, 92%, 93%, 94%, 95%, 96%, 97%, 98%, or 99% sequence identity to SEQ ID NO:6 or 7.

In some embodiments, the multispecific antibody further comprises a first linker between the anti-BCMA VHH and the anti-CD3 VH. In some embodiments, the first linker comprises the amino acid sequence of SEQ ID NO:83 or 84.

In some embodiments, the first anti-CD19 VHH and the second anti-CD19 VHH each, independently, comprises the amino acid sequence of SEQ ID NO:1 or 2. In some embodiments, the first anti-CD19 VHH and the second anti-CD19 VHH each, independently, comprises an amino acid sequence having at least 90%, 91%, 92%, 93%, 94%, 95%, 96%, 97%, 98%, or 99% sequence identity to SEQ ID NO:1 or 2.

In some embodiments, the multispecific antibody further comprises a second linker between the first anti-CD19 VHH and the anti-CD3 VL. In some embodiments, the second linker comprises the amino acid sequence of SEQ ID NO:83 or 84.

In some embodiments, the anti-CD3 VH comprises the amino acid sequence of SEQ ID NO:15 and the anti-CD3 VL comprises the amino acid sequence of SEQ ID NO:19, 20 or 21. In some embodiments, the anti-CD3 VH comprises an amino acid sequence having at least 90%, 91%, 92%, 93%, 94%, 95%, 96%, 97%, 98%, or 99% sequence identity to SEQ ID NO:15 and the anti-CD3 VL comprises an amino acid sequence having at least 90%, 91%, 92%, 93%, 94%, 95%, 96%, 97%, 98%, or 99% sequence identity to SEQ ID NO: 19, 20, or 21.

In some embodiments, the CH1, CH2, and CH3 of the first chain, collectively, comprise the amino acid sequence of SEQ ID NO:57. In some embodiments, the CL comprises the amino acid sequence of SEQ ID NO:71 or 72. In some embodiments, the CH2 and CH3 of the third chain, collectively, comprise the amino acid sequence of SEQ ID NO:58.

In some embodiments, the first chain comprises the amino acid sequence of SEQ ID NO:118 or an amino acid sequence having at least 90%, 91%, 92%, 93%, 94%, 95%, 96%, 97%, 98%, or 99% sequence identity to SEQ ID NO:118. In some embodiments, the second chain comprises the amino acid sequence of SEQ ID NO:119 or an amino acid sequence having at least 90%, 91%, 92%, 93%, 94%, 95%, 96%, 97%, 98%, or 99% sequence identity to SEQ ID NO:119. In some embodiments, the third chain comprises the amino acid sequence of SEQ ID NO:117 or an amino acid sequence having at least 90%, 91%, 92%, 93%, 94%, 95%, 96%, 97%, 98%, or 99% sequence identity to SEQ ID NO:117.

In some embodiments, the first chain, the second chain, and the third chain comprise, respectively, the amino acid sequences of SEQ ID NOs:118, 119 and 117.

Also provided is one or more polynucleotides encoding the multispecific antibody of the present disclosure.

Another embodiment of the present disclosure provides an anti-BCMA single domain antibody (VHH) comprising a CDR1, CDR2 and CDR3 comprising, respectively, the amino acid sequences of SEQ ID NOs:8, 9 and 10. Another embodiment of the present disclosure provides an anti-BCMA single domain antibody (VHH) comprising a CDR1, CDR2 and CDR3 comprising, respectively, the amino acid sequences of SEQ ID NOs:12, 13 and 14.

In some embodiments, the anti-BCMA VHH comprises an amino acid sequence having at least 90%, 91%, 92%, 93%, 94%, 95%, 96%, 97%, 98%, or 99% sequence identity to SEQ ID NO: 6 or 7. In some embodiments, the anti-BCMA VHH comprises the amino acid sequence of SEQ ID NO:6 or 7.In some embodiments, the anti-BCMA VHH comprises an amino acid sequence having at least 90%, 91%, 92%, 93%, 94%, 95%, 96%, 97%, 98%, or 99% sequence identity to SEQ ID NO:11. In some embodiments, the anti-BCMA VHH comprises the amino acid sequence of SEQ ID NO:11.

Another embodiment of the present disclosure provides an anti-CD19 single domain antibody (VHH) comprising a CDR1, CDR2 and CDR3 comprising, respectively, the amino acid sequences of SEQ ID NOs:3, 4, and 5.

In some embodiments, the anti-CD19 VHH comprises an amino acid sequence having at least 90%, 91%, 92%, 93%, 94%, 95%, 96%, 97%, 98%, or 99% sequence identity to SEQ ID NO:1 or 2. In some embodiments, the anti-CD19 VHH comprises the amino acid sequence of SEQ ID NO:1 or 2.

Also provided herein is a multispecific antibody comprising: (a) a first chain comprising an anti-BCMA single domain antibody (VHH), an anti-CD3 heavy chain variable region (VH), a CH1, a CH2, and a CH3; (b) a second chain comprising a first anti-CD19 VHH, an anti-CD3 light chain variable region (VL), and a light chain constant domain (CL); and (c) a third chain comprising a second anti-CD19 VHH, a CH2, and a CH3. In some embodiments, the multispecific antibody induces a reduced cytotoxic T cell response relative to a multispecific antibody targeting CD3, BCMA, and CD19 that is not of the same molecular format.

Single domain antibodies, including VHH molecules, specific to BCMA and CD19 are provided elsewhere herein and are known in the art. Examples of anti-BCMA single domain antibodies are provided, e.g., in WO202305655, WO2022037528, WO2024206569, WO2025099434, and US20240174760, the contents of which are herein incorporated by reference in their entirety. Examples of anti-CD19 single domain antibodies are provided, e.g., in PCT/EP2026/052912, CN110003334, CN114292329, and CN110396128, the contents of which are herein incorporated by reference in their entirety. Further exemplary anti-CD19 and anti-BCMA VHH molecules are provided in Table A of the present disclosure.

Also provided herein is a multispecific antibody comprising: (a) an anti-BCMA VHH, comprising a CDR1, CDR2, and CDR3 according, respectively, to the amino acid sequences of SEQ ID NOs:8, 9, and 10; (b) at least one anti-CD19 VHH, comprising a CDR1, CDR2, and CDR3 according, respectively, to the amino acid sequences of SEQ ID NOs:3, 4, and 5; (c) an anti-CD3 antibody or antigen-binding fragment thereof, comprising a VH and a VL, wherein the VH comprises a H-CDR1, H-CDR2, and H-CDR3, and the VL comprises a L-CDR1, L-CDR2, and L-CDR3, and the H-CDR1, H-CDR2, H-CDR3, L-CDR1, L-CDR2, and L-CDR3 correspond, respectively, to the amino acid sequences of SEQ ID NOs:16, 17, 18, 22, 23, and 24. In some embodiments, the multispecific antibody comprises a first and a second VHH each comprising a CDR1, CDR2, and CDR3 according, respectively, to the amino acid sequences of SEQ ID NOs:3, 4, and 5. These antigen-binding polypeptides have benefits when combined with each other, whether in the same format as Ab101 (and Ab101-1) of FIG. 1, or whether combined in alternative formats.

Methods of treating an autoimmune disease or cancer in a patient in need thereof are also provided, comprising administering to the patient the multispecific antibody or the one or more polynucleotides.

In some embodiments, the cancer is chosen from bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, esophageal cancer, ovarian cancer, renal cancer, melanoma, prostate cancer and thyroid cancer. In some embodiments, the cancer is lymphoma or leukemia.

In some embodiments, the autoimmune disease is chosen from MS (multiple sclerosis), NMOSD (neuromyelitis optica spectrum disorder), NMDAR encephalitis, CIPD (Chronic Inflammatory Demyelinating Polyneurophathy), myasthenia gravis, rheumatoid arthritis, scleroderma or systemic sclerosis, IIM (idiopathic inflammatory myopathy), SLE (Lupus nephritis), pemphigus vulgaris, hidradenitis suppurativa, thrombocytopenic purpura, antiphospholipid syndrome, autoimmune hemolytic anemia, alopecia areata, autoimmune hepatitis, dermatomyositis, diabetes (type 1), celiac disease, autoimmune juvenile idiopathic arthritis, glomerulonephritis, Graves' disease, Guillain-Barré syndrome, idiopathic thrombocytopenic purpura, autoimmune myocarditis, pemphigus, pemphigoid, pernicious anemia, polyarteritis nodosa, polymyositis, primary biliary cirrhosis, psoriasis, Sjögren's syndrome, autoimmune thyroiditis, Hashimoto's thyroiditis, autoimmune uveitis, vitiligo, and granulomatosis with polyangiitis (Wegener's).

### ENUMERATED EMBODIMENTS

The present disclosure relates to multispecific antibodies having specificity to CD3, CD19 and BCMA. These multispecific antibodies have a significantly improved therapeutic index and are useful for treating cancers, autoimmune diseases and/or conditions with reduced toxicities.

For example, embodiments of the disclosure include the following:

Embodiment 1. A multispecific antibody comprising: (a) a first chain comprising an anti-BCMA single domain antibody (VHH), an anti-CD3 heavy chain variable region (VH), a CH1, a CH2, and a CH3; (b) a second chain comprising a first anti-CD19 VHH, an anti-CD3 light chain variable region (VL), and a light chain constant domain (CL); and (c) a third chain comprising a second anti-CD19 VHH, a CH2, and a CH3, wherein (i) the anti-BCMA VHH comprises a CDR1, CDR2, and CDR3 according, respectively, to the amino acid sequences of SEQ ID NOs:8, 9, and 10; (ii) the first and second anti-CD19 VHH each comprise a CDR1, CDR2, and CDR3 according, respectively, to the amino acid sequences of SEQ ID NOs:3, 4, and 5; (iii) the anti-CD3 VH comprises a H-CDR1, H-CDR2, and H-CDR3, the anti-CD3 VL comprises a L-CDR1, L-CDR2, and L-CDR3, and the H-CDR1, H-CDR2, H-CDR3, L-CDR1, L-CDR2, and L-CDR3 according, respectively, to the amino acid sequences of SEQ ID NOs:16, 17, 18, 22, 23, and 24.

Embodiment 2. The multispecific antibody of embodiment 1, wherein the anti-BCMA VHH is N-terminal to the anti-CD3 VH, the first anti-CD19 VHH is N-terminal to the anti-CD3 VL, and the second anti-CD19 VHH is N-terminal to the CH2 of the third chain.

Embodiment 3. The multispecific antibody of embodiment 1 or 2, wherein the anti-BCMA VHH comprises the amino acid sequence of SEQ ID NO:6 or 7 or an amino acid sequence having at least 90%, 91%, 92%, 93%, 94%, 95%, 96%, 97%, 98%, or 99% sequence identity to SEQ ID NO:6 or 7.

Embodiment 4. The multispecific antibody of any one of embodiments 1-3, further comprising a first linker between the anti-BCMA VHH and the anti-CD3 VH.

Embodiment 5. The multispecific antibody of embodiment 4, wherein the first linker comprises the amino acid sequence of SEQ ID NO:83 or 84.

Embodiment 6. The multispecific antibody of any one of embodiments 1-5, wherein the first anti-CD19 VHH and the second anti-CD19 VHH each, independently, comprises the amino acid sequence of SEQ ID NO:1 or 2 or an amino acid sequence having at least 90%, 91%, 92%, 93%, 94%, 95%, 96%, 97%, 98%, or 99% sequence identity to SEQ ID NO:1 or 2.

Embodiment 7. The multispecific antibody of any one of embodiments 1-6, further comprising a second linker between the first anti-CD19 VHH and the anti-CD3 VL.

Embodiment 8. The multispecific antibody of embodiment 7, wherein the second linker comprises the amino acid sequence of SEQ ID NO:83 or 84.

Embodiment 9. The multispecific antibody of any one of embodiments 1-8, wherein (a) the anti-CD3 VH comprises the amino acid sequence of SEQ ID NO:15 or an amino acid sequence having at least 90%, 91%, 92%, 93%, 94%, 95%, 96%, 97%, 98%, or 99% sequence identity to SEQ ID NO:15, and (b) the anti-CD3 VL comprises the amino acid sequence of SEQ ID NO:19, 20, or 21 or an amino acid sequence having at least 90%, 91%, 92%, 93%, 94%, 95%, 96%, 97%, 98%, or 99% sequence identity to SEQ ID NO:19, 20, or 21.

Embodiment 10. The multispecific antibody of any one of embodiments 1-9, wherein the CH1, CH2, and CH3 of the first chain, collectively, comprise the amino acid sequence of SEQ ID NO:57.

Embodiment 11. The multispecific antibody of any one of embodiments 1-10, wherein the CL comprises the amino acid sequence of SEQ ID NO:71 or 72.

Embodiment 12. The multispecific antibody of any one of embodiments 1-11, wherein the CH2 and CH3 of the third chain, collectively, comprise the amino acid sequence of SEQ ID NO:58.

Embodiment 13. The multispecific antibody of any one of embodiments 1-12, wherein the first chain comprises the amino acid sequence of SEQ ID NO:118 or an amino acid sequence having at least 90%, 91%, 92%, 93%, 94%, 95%, 96%, 97%, 98%, or 99% sequence identity to SEQ ID NO:118.

Embodiment 14. The multispecific antibody of any one of embodiments 1-13, wherein the second chain comprises the amino acid sequence of SEQ ID NO:119 or an amino acid sequence having at least 90%, 91%, 92%, 93%, 94%, 95%, 96%, 97%, 98%, or 99% sequence identity to SEQ ID NO:119.

Embodiment 15. The multispecific antibody of any one of embodiments 1-14, wherein the third chain comprises the amino acid sequence of SEQ ID NO:117 or an amino acid sequence having at least 90%, 91%, 92%, 93%, 94%, 95%, 96%, 97%, 98%, or 99% sequence identity to SEQ ID NO:117.

Embodiment 16. The multispecific antibody of any one of embodiments 1-15, wherein the first chain, the second chain, and the third chain comprise, respectively, the amino acid sequences of SEQ ID NOs:118, 119, and 117.

Embodiment 17. The multispecific antibody of any one of embodiments 1-15, wherein the first chain, the second chain, and the third chain comprise, respectively, the amino acid sequences of SEQ ID NOs:123, 119, and 122.

Embodiment 18. A multispecific antibody comprising: (a) a first chain comprising an anti-BCMA single domain antibody (VHH), an anti-CD3 heavy chain variable region (VH), a CH1, a CH2, and a CH3; (b) a second chain comprising a first anti-CD19 VHH, an anti-CD3 light chain variable region (VL), and a light chain constant domain (CL); and (c) a third chain comprising a second anti-CD19 VHH, a CH2, and a CH3.

Embodiment 19. The multispecific antibody of embodiment 18, wherein the anti-BCMA VHH is N-terminal to the anti-CD3 VH.

Embodiment 20. The multispecific antibody of any one of embodiments 18-19, wherein the first anti-CD19 VHH is N-terminal to the anti-CD3 VL.

Embodiment 21. The multispecific antibody of any one of embodiments 18-20, wherein the second anti-CD19 VHH is N-terminal to the CH2 of the third chain.

Embodiment 22. The multispecific antibody of any one of embodiments 18-21, further comprising a first linker between the anti-BCMA VHH and the anti-CD3 VH.

Embodiment 23. The multispecific antibody of any one of embodiments 18-22, further comprising a second linker between the first anti-CD19 VHH and the anti-CD3 VL.

Embodiment 24. The multispecific antibody of any one of embodiments 18-23, wherein the multispecific antibody induces a reduced cytotoxic T cell response relative to a multispecific antibody with the same domains and that does not have an anti-BCMA VHH and/or anti-CD19 VHH N-terminal to the anti-CD3 VH and/or VL.

Embodiment 25. A multispecific antibody comprising: (a) an anti-BCMA VHH, comprising a CDR1, CDR2, and CDR3 according, respectively, to the amino acid sequences of SEQ ID NOs:8, 9, and 10; (b) at least one anti-CD19 VHH, comprising a CDR1, CDR2, and CDR3 according, respectively, to the amino acid sequences of SEQ ID NOs:3, 4, and 5; (c) an anti-CD3 antibody or antigen-binding fragment thereof, comprising a VH and a VL, wherein the VH comprises a H-CDR1, H-CDR2, and H-CDR3, and the VL comprises a L-CDR1, L-CDR2, and L-CDR3, and the H-CDR1, H-CDR2, H-CDR3, L-CDR1, L-CDR2, and L-CDR3 correspond, respectively, to the amino acid sequences of SEQ ID NOs:16, 17, 18, 22, 23, and 24.

Embodiment 26. The multispecific antibody of embodiment 25, wherein the multispecific antibody comprises a first and a second anti-CD19 VHH each comprising a CDR1, CDR2, and CDR3 according, respectively, to the amino acid sequences of SEQ ID NOs:3, 4, and 5.

Embodiment 27. The multispecific antibody of any one of embodiments 25-26, wherein the anti-BCMA VHH comprises the amino acid sequence of SEQ ID NO:6 or 7 or an amino acid sequence having at least 90%, 91%, 92%, 93%, 94%, 95%, 96%, 97%, 98%, or 99% sequence identity to SEQ ID NO:6 or 7.

Embodiment 28. The multispecific antibody any one of embodiments 25-27, wherein the first anti-CD19 VHH and the second anti-CD19 VHH each, independently, comprises the amino acid sequence of SEQ ID NO:1 or 2 or an amino acid sequence having at least 90%, 91%, 92%, 93%, 94%, 95%, 96%, 97%, 98%, or 99% sequence identity to SEQ ID NO:1 or 2.

Embodiment 29. The multispecific antibody of any one of embodiments 25-28, wherein (a) the anti-CD3 VH comprises the amino acid sequence of SEQ ID NO:15 or an amino acid sequence having at least 90%, 91%, 92%, 93%, 94%, 95%, 96%, 97%, 98%, or 99% sequence identity to SEQ ID NO:15, and (b) the anti-CD3 VL comprises the amino acid sequence of SEQ ID NO:19, 20, or 21 or an amino acid sequence having at least 90%, 91%, 92%, 93%, 94%, 95%, 96%, 97%, 98%, or 99% sequence identity to SEQ ID NO:19, 20, or 21.

Embodiment 30. The multispecific antibody of any one of embodiments 18-29, wherein the multispecific antibody comprises at least one polypeptide chain comprising a CH2 and a CH3 domain.

Embodiment 31. The multispecific antibody of any one of embodiments 18-30, wherein the multispecific antibody comprises at least two polypeptide chains each comprising a CH2 and a CH3 domain.

Embodiment 32. The multispecific antibody of any one of embodiments 18-31, wherein the multispecific antibody comprises at least one polypeptide chain comprising a CH1, CH2, and a CH3 domain,

Embodiment 33. The multispecific antibody of any one of embodiments 18-32, wherein the multispecific antibody comprises at least one CL domain.

Embodiment 34. The multispecific antibody of any one of embodiments 18-33, wherein the polypeptide chain comprising a CH1, CH2, and CH3 comprises the amino acid sequence of SEQ ID NO:57.

Embodiment 35. The multispecific antibody of any one of embodiments 18-34, wherein the CL comprises the amino acid sequence of SEQ ID NO:71 or 72.

Embodiment 36. The multispecific of any one of embodiments 18-35, wherein the polypeptide chain comprising a CH2 and CH3 comprises the amino acid sequence of SEQ ID NO:58.

Embodiment 37. A composition comprising the multispecific antibody of any one of embodiments 1-36, and a pharmaceutically acceptable carrier.

Embodiment 38. One or more polynucleotides encoding the multispecific antibody of any one of embodiments 1-36.

Embodiment 39. An isolated cell comprising the one or more polynucleotides of embodiment 38.

Embodiment 40. A method of treating an autoimmune disease or cancer in a patient in need thereof, comprising administering to the patient the multispecific antibody of any one of embodiments 1-36, the composition of embodiment 37, or the one or more polynucleotides of embodiment 38.

Embodiment 41. The multispecific antibody of any one of embodiments 1-36, the composition of embodiment 37, or the one or more polynucleotides of embodiment 38, for use as a medicament.

Embodiment 42. The multispecific antibody of any one of embodiments 1-36, the composition of embodiment 38, or the one or more polynucleotides of embodiment 38, for use in the treatment of an autoimmune disease or cancer in a patient in need thereof.

Embodiment 43. Use of the multispecific antibody of any one of embodiments 1-36, the composition of embodiment 37, or the one or more polynucleotides of embodiment 38 for treating autoimmune disease or cancer in a patient in need thereof.

Embodiment 44. The method, multispecific antibody for use, composition for use, one or more polynucleotides for use, or the use of any one of embodiments 40 and 42-43, wherein the cancer is chosen from of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, esophageal cancer, ovarian cancer, renal cancer, melanoma, prostate cancer and thyroid cancer.

Embodiment 45. The method, multispecific antibody for use, composition for use, one or more polynucleotides for use, or the use of embodiment 44, wherein the cancer is lymphoma or leukemia.

Embodiment 46. The method, multispecific antibody for use, composition for use, one or more polynucleotides for use, or the use of embodiment 40 and 42-43, wherein the autoimmune disease is chosen from MS (multiple sclerosis), NMOSD (neuromyelitis optica spectrum disorder), NMDAR encephalitis, CIPD (Chronic Inflammatory Demyelinating Polyneurophathy), myasthenia gravis, rheumatoid arthritis, scleroderma or systemic sclerosis, IIM (idiopathic inflammatory myopathy), SLE (Lupus nephritis), pemphigus vulgaris, hidradenitis suppurativa, thrombocytopenic purpura, antiphospholipid syndrome, autoimmune hemolytic anemia, alopecia areata, autoimmune hepatitis, dermatomyositis, diabetes (type 1), celiac disease, autoimmune juvenile idiopathic arthritis, glomerulonephritis, Graves' disease, Guillain-Barré syndrome, idiopathic thrombocytopenic purpura, autoimmune myocarditis, pemphigus, pemphigoid, pernicious anemia, polyarteritis nodosa, polymyositis, primary biliary cirrhosis, psoriasis, Sjögren's syndrome, autoimmune thyroiditis, Hashimoto's thyroiditis, autoimmune uveitis, vitiligo, and granulomatosis with polyangiitis (Wegener's).

### BRIEF DESCRIPTION OF THE DRAWINGS

FIGS. 1A-H shows various multispecific antibody formats.

FIG. 2 shows human B cell killing by the tested antibodies.

FIG. 3 shows monkey B cell killing by the tested antibodies.

FIGS. 4A-C shows human B cell killing by the tested antibodies.

FIGS. 5A-B shows cytokine release by the tested antibodies.

FIG. 6 shows B cell depletion by the tested antibodies.

FIG. 7A-F shows ELISA binding to target proteins by Ab101.

FIG. 8A-B shows killing of BCMA+ B cells by Ab101.

FIG. 9A-B shows human and cyno B cell killing by Ab101.

FIG. 10A-D shows cytokine release by Ab101.

FIG. 11 shows B cell depletion by Ab101-1.

FIG. 12 shows binding curves of Ab101-1 and teclistamab to human CD4+ T cells.

FIG. 13 shows a binding curve of Ab101-1 to human CD4+ T cells.

FIG. 14 shows binding curves of Ab101-1 and teclistamab to human CD8+ T cells.

FIG. 15 shows a binding curve of Ab101-1 to human CD8+ T cells.

FIG. 16 shows binding curves of Ab101-1 and teclistamab to human CD20+ B cells.

FIG. 17 shows binding curves of Ab101-1 and teclistamab to cyno CD4+ T cells.

FIG. 18 shows a binding curve of Ab101-1 to cyno CD4+ T cells.

FIG. 19 shows binding curves of Ab101-1 and teclistamab to cyno CD8+ T cells.

FIG. 20 shows a binding curve of Ab101-1 to cyno CD8+ T cells.

FIG. 21 shows binding curves of Ab101-1 and teclistamab to cyno CD20+ B cells.

FIG. 22 shows an exemplary flow cytometry gating strategy used in Example 15.

FIGS. 23A-D show B cell subset depletion following treatment with blinatumomab, teclistamab, duvortuxizumab, Ab101-1 or an Isotype control. Values are normalized from 10 donor PBMCs. Data points are normalized against cell counts from no antibody treatment wells and represented as mean±SEM.

FIGS. 24A-B show plasmablast and plasma cell depletion following treatment with blinatumomab, teclistamab, duvortuxizumab, Ab101-1 or an isotype control. Values normalized from donors 5, 6 and 10. Data points are normalized against cell counts from no antibody treatment wells and represented as mean±SEM.

FIGS. 25A-C show CD8+CD25+ activated T cell profile following treatment with blinatumomab, teclistamab, duvortuxizumab, Ab101-1 or an isotype control. Data are represented in terms of A) Cell counts (B) Frequency of total CD8+ T cells or (C) Geometric mean fluorescence intensity (gMFI) of CD25 signal, averaged from 10 donor PBMCs. Data points are normalized against cell counts from no antibody treatment wells and represented as mean±SEM.

FIGS. 26A-F show cytokine concentrations following treatment with blinatumomab, teclistamab, duvortuxizumab, Ab101-1 or an isotype control. Data are averaged from 10 donor PBMCs. Data points are normalized against cytokine concentration from 200 nM duvortuxizumab treatment wells and represented as mean±SEM.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

As used herein, the term “sequence identity” refers to the percentage of exact nucleotide or amino acid residue matches between two or more sequences when aligned for maximum correspondence over the full length of the reference sequence. The percentage of identity between two sequences is a function of the number of identical positions shared by the sequences, considering the number of gaps, and the length of each gap, which need to be introduced for optimal alignment. “Sequence identity” is strictly distinguished from “sequence similarity,” which additionally accounts for conservative amino acid substitutions. One method for determining the percentage of sequence identity between two amino acid or nucleotide sequences uses the Needleman-Wunsch global alignment algorithm (Needleman and Wunsch, 1970, J. Mol. Biol. 48: 443-453), as implemented in the EMBOSS Needle program. Other methods are well known in the art. As further illustration, for amino acid sequence comparisons, the default parameters can be: a BLOSUM62 scoring matrix, a gap opening penalty of 10.0, and a gap extension penalty of 0.5. For nucleotide sequence comparisons, the default parameters can be the EDNAFULL scoring matrix, a gap opening penalty of 10.0, and a gap extension penalty of 0.5.

Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The expression “single domain antibodies” (sdAbs) or “single variable domain (SVD) antibodies” generally refers to antibodies in which a single variable domain (VH or VL) can confer antigen binding. In other words, the single variable domain does not need to interact with another variable domain in order to recognize the target antigen. Examples of single domain antibodies include those derived from camelids (lamas and camels) and cartilaginous fish (e.g., nurse sharks) and those derived from recombinant methods from humans and mouse antibodies (Nature (1989) 341:544-546; Dev Comp Immunol (2006) 30:43-56; Trend Biochem Sci (2001) 26:230-235; Trends Biotechnol (2003):21:484-490; WO 2005/035572; WO 03/035694; Febs Lett (1994) 339:285-290; WO00/29004; WO 02/051870). When the sdAb contains only a heavy chain, it can be exchangeably used with “VHH” or “single heavy chain variable domain antibody” or “nanobody”.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” also includes any synthetic or genetically engineered protein that acts like an antibody by binding to a specific antigen to form a complex, such as diabodies, triabodies, and single-domain antibodies. Separately, the broader term “antigen-binding moiety” encompasses both antibody fragments as well as non-proteinaceous or non-antibody scaffolds, such as aptamers and spiegelmers.

A “Fab” with regard to an antibody refers to a monovalent antigen-binding fragment of the antibody consisting of a single light chain (both variable and constant regions) bound to the variable region and first constant region of a single heavy chain by a disulfide bond. Fab can be obtained by papain digestion of an antibody at the residues proximal to the N-terminus of the disulfide bond between the heavy chains of the hinge region.

A “Fab′” refers to a Fab fragment that includes a portion of the hinge region, which can be obtained by pepsin digestion of an antibody at the residues proximal to the C-terminus of the disulfide bond between the heavy chains of the hinge region and thus is different from Fab in a small number of residues (including one or more cysteines) in the hinge region.

A “F(ab)₂” refers to a dimer of Fab′ that comprises two light chains and part of the two heavy chains.

A “single-chain variable fragment” or “scFv” refers to a fusion protein of the variable regions of the heavy (V_{H}) and light chains (V_{L}) of immunoglobulins. In some aspects, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

A “full-length antibody” typically comprises two heavy chains and two light chains except in certain multispecific formats. The variable regions of the light and heavy chains are responsible for antigen binding. The variable domains of the heavy chain and light chain may be referred to as “VH” and “VL”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia or Al-Lazikani, or IMGT (Al-Lazikani 1997; Chothia 1985; Chothia 1987; Chothia 1989; Kabat 1987; Kabat 1991, Zhu, Z.; Olson, K. S.; Magliery, T. J. 50 Years of Antibody Numbering Schemes: A Statistical and Structural Evaluation Reveals Key Differences and Limitations. Antibodies 2024, 13, 99.). However, unless otherwise explicitly stated in the claims, the term “CDR” as used herein refers to the complementarity determining regions as defined by the Kabat numbering system. The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as lgG1 (γ1 heavy chain), lgG2 (γ2 heavy chain), lgG3 (γ3 heavy chain), lgG4 (γ4 heavy chain), lgA1 (α1 heavy chain), or lgA2 (α2 heavy chain).

As used herein, the term “multispecific antibody” refers to an antibody, antigen-binding polypeptide, or engineered construct that comprises at least two different antigen-binding domains, wherein each antigen-binding domain specifically binds to a different epitope. The distinct epitopes may be present on the same target antigen (i.e., a biparatopic or multiparatopic antibody) or present on entirely distinct target antigens. The term broadly encompasses, but is not limited to, bispecific, trispecific, and tetraspecific antibodies. Multispecific antibodies may take a variety of structural formats, including both symmetric and asymmetric architectures. By way of non-limiting example, multispecific antibodies include formats utilizing “knobs-into-holes” (KiH) engineering, common light chains, CrossMab technology, electrostatically steered Fc domains, Dual-Variable Domain immunoglobulins (DVD-Ig), IgG-scFv or IgG-Fab fusions, half-antibody dimers, and tandem single-chain variable fragments (e.g., bispecific T-cell engagers). The term expressly encompasses multispecific constructs comprising a full or partial fragment crystallizable (Fc) region, as well as fragment-based multispecifics lacking an Fc region (e.g., F(ab′)2, diabodies, and tribodies).

The term “half-antibody” as used herein refers to one immunoglobulin heavy chain associated with one immunoglobulin light chain. One skilled in the art will readily appreciate that a half-antibody may encompass a fragment thereof and may also have an antigen binding domain consisting of a single variable domain, e.g., originating from a camelidae. In certain embodiments, the half antibody comprises a Fab′ and a Fc domain. The Fab′ is fused to N-terminus of the Fc domain.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VK) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually comprise the carboxy-terminus of the heavy and light chain, respectively.

As indicated above, the variable region allows the antibody to selectively recognize and specifically bind epitopes on antigens. That is, the VK domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. More specifically, the antigen-binding site is defined by three CDRs on each of the VH and VK chains (i.e. H-CDR1, H-CDR2, H-CDR3, L-CDR1, L-CDR2 and L-CDR3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see “Sequences of Proteins of Immunological Interest,” Kabat, E., et al., U.S. Department of Health and Human Services, (1983); and Chothia and Lesk, J. MoI. Biol., 196:901-917 (1987)).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. MoI. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

| | Kabat | Chothia |
|---|---|---|
| H-CDR1 | 31-35 | 26-32 |
| H-CDR2 | 50-65 | 52-58 |
| H-CDR3 | 95-102 | 95-102 |
| L-CDR1 | 24-34 | 26-32 |
| L-CDR2 | 50-56 | 50-52 |
| L-CDR3 | 89-97 | 91-96 |

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids. CDR-L1 begins at approximately residue 24 (i.e., following a cysteine residue); includes approximately 10-17 residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues.

Unless otherwise stated, the CDR of the VH and VL regions are determined by using the Chothia numbering system.

Antibodies disclosed herein may be from any animal origin including birds and mammals. In certain embodiments, the antibodies are human, murine, donkey, rabbit, goat, guinea pig, camel, llama, horse, or chicken antibodies. In another embodiment, the variable region may be condricthoid in origin (e.g., from sharks).

As used herein, the term “heavy chain constant region” includes amino acid sequences derived from an immunoglobulin heavy chain. A polypeptide comprising a heavy chain constant region comprises at least one of: a CH1 domain, a hinge (e.g., upper, middle, and/or upper hinge region) domain, a CH2 domain, a CH3 domain, or a variant or fragment thereof. For example, an antigen-binding polypeptide for use in the disclosure may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, an antibody for use in the disclosure may lack at least a portion of a CH2 domain (e.g., all or part of a CH2 domain). As set forth above, it will be understood by one of ordinary skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CH1 domain derived from an IgG₁ molecule and a hinge region derived from an IgG₃ molecule. In another example, a heavy chain constant region can comprise a hinge region derived, in part, from an IgG₁ molecule and, in part, from an IgG₃ molecule. In another example, a heavy chain portion can comprise a chimeric hinge derived, in part, from an IgG₁ molecule and, in part, from an IgG₄ molecule.

As used herein, the term “light chain constant region” (CL) includes amino acid sequences derived from antibody light chain. In some embodiments, the light chain constant region comprises at least one of a constant kappa domain or constant lambda domain.

A “light chain-heavy chain pair” refers to the collection of a light chain and heavy chain that can form a dimer through a disulfide bond between the CL domain of the light chain and the CH1 domain of the heavy chain.

As previously indicated, the subunit structures and three-dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat et al., U.S. Dept. of Health and Human Services, “Sequences of Proteins of Immunological Interest” (1983). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen-binding regions to move independently. Hinge regions can be subdivided into three distinct domains: upper, middle, and upper hinge domains (Roux et al., J. Immunol 161:4083 (1998)). In certain embodiments, the hinge region can include the amino acid sequence of SEQ ID NO: 47.

The term “Fc region”, “Fc domain” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CK regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant disclosure) is obtained from a second species. In certain embodiments the target binding region or site will be from a non-human source (e.g. mouse or primate) and the constant region is human.

“Humanized antibody” is used herein to describe an antibody that comprises heavy and light chain variable region sequences from a non-human species (e.g. a mouse) but in which at least a portion of the VH and/or VL sequence has been altered to be more “human-like,” i.e., more similar to human germline variable sequences. A “humanized antibody” is an antibody or a variant, derivative, analog, or fragment thereof, which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having the amino acid sequence at least 80%, at least 85%, at least 90%, at least 95%, at least 98%, or at least 99% identical to the amino acid sequence of a non-human antibody CDR. A humanized antibody comprises substantially all of at least one, and typically two, variable domains (Fab, Fab′, F(ab′)₂, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

The term “epitope” as used herein refers to the specific group of atoms or amino acid residues on an antigen that directly contact the paratope of an antibody or antibody fragment. An epitope may be linear (sequential) or conformational (discontinuous).. Two antibodies or antibody moieties may bind the same epitope within an antigen if they exhibit competitive binding for the antigen. Separately, two antibodies or antibody moieties are said to “compete for binding” or “cross-block” each other if they sterically hinder one another's binding to the target antigen. Competitive binding indicates that the antibodies belong to the same binding bin, which may occur whether they bind the exact same epitope or overlapping/adjacent epitopes.

As used herein, “percent humanization” is calculated by determining the number of framework amino acid differences (i.e., non-CDR difference) between the humanized domain and the germline domain, subtracting that number from the total number of amino acids, and then dividing that by the total number of amino acids and multiplying by 100.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

Multispecific Antibodies with Reduced T Cell Activation

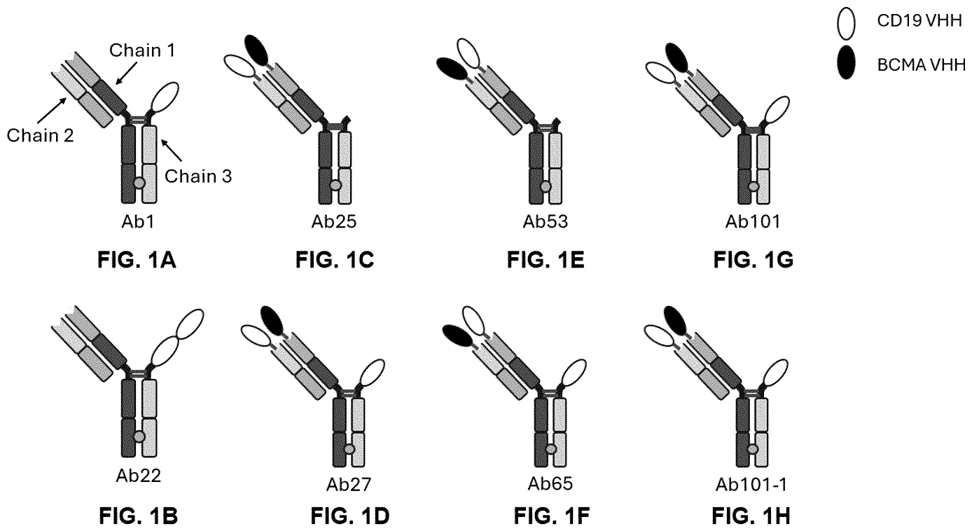

The present disclosure provides a new format of multispecific antibodies. As illustrated in antibody Ab101 (and Ab101-1) of FIG. 1, the new format includes a monovalent anti-CD3 portion that includes an anti-CD3 Fab fused to one of the two chains of an Fc fragment. Three single domain antibodies (VHH) are individually fused to the N-termini of the three peptide chains of the monovalent anti-CD3 portion.

The accompanying examples demonstrated that Ab101, as well as its close variant Ab101-1, exhibited significantly improved B cell killing but reduce cytokine release, as compared to other multispecific antibody formats and benchmark antibody blinatumomab. As cytokine release has been a significant safety concern for immunotherapies, which have limited their effectiveness, these new multispecific antibodies provide a significant breakthrough, in terms of therapeutic efficacy and safety (thus therapeutic index), over the conventional technologies.

The Ab101 and Ab101-1 molecules include a single anti-CD3 binding fragment (the Fab). This anti-CD3 binding fragment can be derived from SP34. There are already many known humanized versions of SP34. For instance, in Liu et al., “An adapted consensus protein design strategy for identifying globally optimal biotherapeutics,” MABS 2022, VOL. 14, NO. 1, e2073632 (13 pages), eight humanized SP34 antibodies were discussed. Moreover, WO2024129897A2 describes various humanized VH and VL sequences of SP34. These humanized versions have highly similar sequences and are believed to be suitable for the instantly multispecific antibodies.

Such a monovalent anti-CD3 core, therefore, can serve as a scaffold for multispecific antibodies that have desired anti-CD3 activities for therapeutic uses. Moreover, the distance of the VHH to the VH/VL of the Fab can be fine-tuned to achieve desired anti-CD3 activities. The three VHH, on the other hand, provide additional binding specificities to targets such as tumor antigens and immune cell markers.

One embodiment of the present disclosure provides a multispecific antibody which includes, among others, (a) a first chain comprising an anti-BCMA single domain antibody (VHH), an anti-CD3 heavy chain variable region (VH), a CH1, a CH2, and a CH3; (b) a second chain comprising a first anti-CD19 VHH, an anti-CD3 light chain variable region (VL), and a CL; and (c) a third chain comprising a second anti-CD19 VHH, a CH2, and a CH3.

Another embodiment of the present disclosure provides a multispecific antibody comprising antigen binding polypeptides specifically targeting BCMA, CD19, and CD3 without requiring the sequences to be arranged in the Ab101 (and AB101-1) format of FIG. 1. The following sequences may serve as the basis both for multispecific antibodies in the newly described format and those in other formats. Alternative multispecific formats are known in the art.

Example anti-BCMA VHHs are provided and tested in this disclosure. In some embodiments, the anti-BCMA VHH includes a CDR1, CDR2, and CDR3 which include, respectively, the amino acid sequences of SEQ ID NO:8, 9, and 10. In some embodiments, the anti-BCMA VHH includes an amino acid sequence that includes the recited CDRs and is at least 60%, 65%, 70%, 75%, 80%, 85%, 90%, 95%, 98% or 99%, or 100% identical to SEQ ID NO:6. In some embodiments, the anti-BCMA VHH includes an amino acid sequence that includes the recited CDRs and is at least 60%, 65%, 70%, 75%, 80%, 85%, 90%, 95%, 98% or 99%, or 100% identical to SEQ ID NO:7.

In some embodiments, the anti-BCMA VHH includes a CDR1, CDR2, and CDR3 which include, respectively, the amino acid sequences of SEQ ID NOs:12, 13, and 14. In some embodiments, the anti-BCMA VHH includes an amino acid sequence that includes the recited CDRs and is at least 60%, 65%, 70%, 75%, 80%, 85%, 90%, 95%, 98% or 99%, or 100% identical to SEQ ID NO:11.

Single domain antibodies, including VHH molecules, specific to BCMA are known in the art. Examples of anti-BCMA single domain antibodies are provided, e.g., in WO202305655, WO2022037528, WO2024206569, WO2025099434, and US20240174760, the contents of which are herein incorporated by reference in their entirety.

Example anti-CD19 VHHs are provided and tested in this disclosure. In some embodiments, the first anti-CD19 VHH includes a CDR1, CDR2, and CDR3 which include, respectively, the amino acid sequences of SEQ ID NO:3, 4, and 5. In some embodiments, the first anti-CD19 VHH includes an amino acid sequence that includes the recited CDRs and is at least 60%, 65%, 70%, 75%, 80%, 85%, 90%, 95%, 98% or 99%, or 100% identical to SEQ ID NO:1. In some embodiments, the first anti-CD19 VHH includes an amino acid sequence that includes the recited CDRs and is at least 60%, 65%, 70%, 75%, 80%, 85%, 90%, 95%, 98% or 99%, or 100% identical to SEQ ID NO:2.

In some embodiments, the second anti-CD19 VHH includes a CDR1, CDR2, and CDR3 which include, respectively, the amino acid sequences of SEQ ID NO:3, 4, and 5. In some embodiments, the second anti-CD19 VHH includes an amino acid sequence that includes the recited CDRs and is at least 60%, 65%, 70%, 75%, 80%, 85%, 90%, 95%, 98% or 99%, or 100% identical to SEQ ID NO:1. In some embodiments, the second anti-CD19 VHH includes an amino acid sequence that includes the recited CDRs and is at least 60%, 65%, 70%, 75%, 80%, 85%, 90%, 95%, 98% or 99%, or 100% identical to SEQ ID NO:2.

Single domain antibodies, including VHH molecules, specific to CD19 are known in the art. Examples of anti-CD19 single domain antibodies are provided, e.g., in PCT/EP2026/052912, CN110003334, CN114292329, and CN110396128, the contents of which are herein incorporated by reference in their entirety.

A SP34-derived anti-CD3 antibody or antigen-binding fragment, which can suitably be included in the presently disclosed multispecific antibodies, may include a VH/VL pair, in which the VH includes a H-CDR1, H-CDR2, and H-CDR3 and the VL includes a L-CDR1, L-CDR2, and L-CDR3. In some embodiments, the H-CDR1, H-CDR2, H-CDR3, L-CDR1, L-CDR2, and L-CDR3 include, respectively, the amino acid sequences of SEQ ID NOs:16, 17, 18, 22, 23, and 24.

In some embodiments, the anti-CD3 VH includes an amino acid that includes the recited H-CDR1, CDR2, and CDR3 and is at least 60%, 65%, 70%, 75%, 80%, 85%, 90%, 95%, 98% or 99%, or 100% identical to SEQ ID NO:15. In some embodiments, the anti-CD3 VL includes an amino acid that includes the recited L-CDR1, CDR2 and CDR3 and is at least 60%, 65%, 70%, 75%, 80%, 85%, 90%, 95%, 98% or 99%, or 100% identical to SEQ ID NO:19, 20, or 21.

In some embodiments, the anti-CD3 VH comprises the amino acid sequence of SEQ ID NO:15 and the VL comprises the amino acid sequence of SEQ ID NO:19. In some embodiments, the anti-CD3 VH comprises the amino acid sequence of SEQ ID NO:15 and the VL comprises the amino acid sequence of SEQ ID NO:20. In some embodiments, the anti-CD3 VH comprises the amino acid sequence of SEQ ID NO:15 and the VL comprises the amino acid sequence of SEQ ID NO:21.

In some embodiments, the anti-BCMA VHH is N-terminal to the anti-CD3 VH, the first anti-CD19 VHH is N-terminal to the anti-CD3 VL, and the second anti-CD19 VHH is N-terminal to the CH2 of the third chain. In other words, in some embodiments, all three recited VHHs are N-terminal the anti-CD3 core/Fc.

In some embodiments, a linker is included between the VHH and the corresponding anti-CD3 VH or VL. For instance, the first chain can include a first linker between the anti-BCMA VHH and the anti-CD3 VH. In some embodiments, the second chain includes a second linker between the anti-CD19 VHH and the anti-CD3 VL.

Linkers within the scope of the present disclosure are characterized in terms of amino acid content, length, rigidity and secondary structure. Linkers within the scope of the present disclosure separate a function polypeptide and another functional polypeptide and allow proper folding and functioning of each domain. In this manner, a linker can be tailored to the particular functional polypeptide and the other functional polypeptide. According to one aspect, functional independence of the structural and fused (heterologous) domains is maximized by a suitable linker to limit steric interference between domains during the export and assembly processes of the bacterial cell. According to an additional aspect, cell stress is minimized by limiting the overall length of the fusion protein. Longer linker sequences and higher induction levels stress the biosynthetic machinery of the cells, inhibiting cell growth and leading to cell lysis in extreme cases.

Peptide linkers within the scope of the present disclosure include amino acid residues. The amino acid residues may be any of the naturally occurring amino acid residues. Amino acid residues may also be synthetic amino acids known to those of skill in the art. Representative amino acids which may be used in linkers include Glycine, Alanine, Valine, Leucine, Isoleucine, Serine, Cysteine, Selenocysteine, Threonine, Methionine, Proline, Phenylalanine, Tyrosine, Tryptophan, Histidine, Lysine, Arginine, Aspartate, Glutamate, Asparagine, and Glutamine.

According to one aspect, the peptide linker length can be any length which may be expressed from a cell, such as a bacterial cell when linking the antibody domains. In some embodiments, the linker has a length of about four to about fifty amino acids. According to one aspect, a peptide linker sequence is a polypeptide sequence of at least 3, 4, 5, 6, 7, 8, 9, 10, 11, 12, 13, 14, 15, 16, 17, 18, 19, 20, 24, 48 or more amino acids.

In some embodiments, the linker sequence comprises a flexible polypeptide, e.g. a polypeptide not having a rigid secondary and/or tertiary structure. In some embodiments, the linker sequence comprises glycine and serine residues. In some embodiments at least 50% of the amino acids comprised by the linker sequence are glycine or serine residues, e.g. at least 50%, at least 60%, at least 70%, at least 80%, at least 90%, at least 95%, or more are glycine or serine residues. In some embodiments, the linker sequence consists of glycine and serine residues.

In certain embodiments, the linker includes partial or full length of the hinge region. In certain embodiments, the fusion between all the antibody moieties and the N-terminus of the Fc domain is via a partial or full length of the hinge region.

Examples of linker sequences are provided in Table C. In some embodiments, at least 50% of the residues in the linker are G or S. In some embodiments, at least 60%, 70%, 80%, 90%, or 100% of the residues in the linker are G or S. In some embodiments, the first linker comprises the amino acid sequence of SEQ ID NO:83. In some embodiments, the second linker comprises the amino acid sequence of SEQ ID NO:84.

In some embodiments, the second anti-CD19 VHH is fused to the N-terminus of one of the chains of the Fc fragment (or the CH2) through a linker. In some embodiments, the linker is all or part of an IgG hinge. Typically, a CH2 starts with A231 (EU numbering).

There may be additional domains or fragments in the multispecific antibodies, without limitation. For instance, there may be one or more antigen-binding fragments (e.g., VHH) fused the N-terminus of any of the first/second/third VHH.

In addition or alternatively, in some embodiments, there may be one or more antigen-binding fragments (e.g., VHH) fused to the C-terminus of either one or both chains of the Fc fragment. Yet in an alternative embodiment, the multispecific antibody does not include an antigen binding fragment that is C-terminal to the Fc fragment.

TABLE A
Antibody Sequences

| | | SEQ ID |
|---|---|---|
| Description | Amino acid sequence | NO: |
| CD19 VHH | QVQLVESGGGLVQDGDSLWLSCAASGAFFGVGIGWFRQAPGKEREFVAAIT | 1 |
| (CDRs | WSGGRTWYTKDVKGRFIISADKPRNTVLLQMNDLKSEDTAVYYCASGDPSG | |
| underlined) | GAGPESVRQYLHWGQGTQVTVSS | |
| CD19 VHH | EVQLVESGGGLVQPGGSLRLSCAASGAFFGVGIGWFRQAPGKGREFVAAIT | 2 |
| humanized | WSGGRTWYTKDVKGRFTISADKSKNTVYLQMNSLRAEDTAVYYCASGDPSG | |
| (CD19 hVHH, | GAGPESVRQYLHWGQGTLVTVSS | |
| CDRs | | |
| underlined) | | |
| CD19 VHH CDR1 | GAFFGV | 3 |
| CD19 VHH CDR2 | TWSGGR | 4 |
| CD19 VHH CDR3 | GDPSGGAGPESVRQYLH | 5 |
| BCMA hVHH1 | EVQLVESGGGLVQPGGSLRLSCATSGFTFSPSAMNWVRQAPGKGLEWVADI | 6 |
| (CDRs | YGDGRAVYADSVKGRFTVSRDNSKNLVYLQMNSLRAEDTAVYVCGVGRYTT | |
| underlined) | TPLSEKLWGQGTLVTVSS | |
| BCMA hVHH2 | EVQLVESGGGLVQPGGSLRLSCATSGFTFSPSAMNWVRQAPGEGLEWVADI | 7 |
| (CDRs | YGDGRAVYADSVKGRFTVSRDNAKKLVYLEMNDLRPEDTAVYVCGVGRYTT | |
| underlined) | TPLSEKLRGQGTLVTVSS | |
| BCMA hVVH1/ | GFTFSPS | 8 |
| 2CDR1 | | |
| BCMA hVVH1/ | YGDGR | 9 |
| 2CDR2 | | |
| BCMA hVVH1/ | GRYTTTPLSEKL | 10 |
| 2CDR3 | | |
| BCMA hVHH3 | QVQLVESGGGLVQPGGSLRLTCVASGSGDNFFSMGWFRQAPGKQREWVAFI | 11 |
| (CDRs | YDQRATLYRDSVKGRFTISRDSAKNTAFLQMNSLKPEDTGVYYCNVILDSG | |
| underlined) | TGDSDRSTYWGQGTQVTVSS | |
| BCMA hVVH3 | GSGDNFF | 12 |
| CDR1 | | |
| BCMA hVVH3 | YDQRA | 13 |
| CDR2 | | |
| BCMA hVVH3 | ILDSGTGDSDRSTY | 14 |
| CDR3 | | |
| CD3 VH (CDRs | EVQLVESGGGLVQPGGSLKLSCAASGFTENTYAMNWVRQASGKGLEWVARI | 15 |
| underlined) | RSKYNNYATYYADSVKDRFTISRDDSLNTLYLQMNSLKTEDTAVYYCVRHG | |
| | NFGNSYVSWFAYWGQGTLVTVSS | |
| CD3 H-CDR1 | TYAMN | 16 |
| CD3 H-CDR2 | RIRSKYNNYATYYADSVKD | 17 |
| CD3 H-CDR3 | HGNFGNSYVSWFAY | 18 |
| CD3 VL (CDRs | QAVVTQEPSLTVSPGGTVTLTCRSSTGAVTTSNYANWVQEKPGQLFRGLIG | 19 |
| underlined) | GTNKRAPWVPARFSGSLLGDKAALTLSGAQPEDEAEYFCALWYSNLWVEGG | |
| | GTKLTVL | |
| CD3 VL2 (CDRs | QAVVTQEPSLTVSPGGTVTLTCRSSTGAVTTSNYANWVQQKPGQAFRGLIG | 20 |
| underlined) | GTNKRAPWTPARFSGSLLGGKAALTLSGAQPEDEAEYFCALWYSNLWVFGG | |
| | GTKLTVL | |
| CD3 VL3 (CDRs | QTVVTQEPSLTVSPGGTVTLTCRSSTGAVTTSNYANWVQEKPGQLFRGLIG | 21 |
| underlined) | GTNKRAPWVPARFSGSLLGDKAALTLSGAQPEDEAEYFCALWYSNLWVFGG | |
| | GTKLTVL | |
| CD3 L-CDR1 | RSSTGAVTTSNYAN | 22 |
| CD3 L-CDR2 | GTNKRAP | 23 |
| CD3 L-CDR3 | ALWYSNLWV | 24 |
| SP34 VH (CDRs | EVQLVESGGGLVQPKGSLKLSCAASGFTENTYAMNWVRQAPGKGLEWVARI | 25 |
| underlined) | RSKYNNYATYYADSVKDRFTISRDDSQSILYLQMNNLKTEDTAMYYCVRHG | |
| | NFGNSYVSWFAYWGQGTLVTVSS | |
| SP34 VL (CDRs | QAVVTQESALTTSPGETVTLTCRSSTGAVTTSNYANWVQEKPDHLFTGLIG | 26 |
| underlined) | GTNKRAPGVPARFSGSLIGDKAALTITGAQTEDEALYFCALWYSNLWVEGG | |
| | GTKLTVL | |

TABLE B
Constant regions

| | | | SEQ ID |
|---|---|---|---|
| Fragment | Mutations | Amino acid sequence | NO: |
| CH2CH3 | L234A, L235A, | DKTHTCPPCPAPEAAGAPSVFLFPPKPKDTLMISRTPEVTCVV | 51 |
| | G237A, T366W | VDVSHEDPEVKFNWYVDGVEVHNAKTKPREEQYNSTYRVVSVL | |
| | | TVLHQDWLNGKEYKCKVSNKALPAPIEKTISKAKGQPREPQVY | |
| | | TLPPSREEMTKNQVSLWCLVKGFYPSDIAVEWESNGQPENNYK | |
| | | TTPPVLDSDGSFFLYSKLTVDKSRWQQGNVFSCSVMHEALHNH | |
| | | YTQKSLSLSPG | |
| C220S- | AK218, L234A, | EPSSDKTHTCPPCPAPEAAGAPSVFLFPPKPKDTLMISRTPEV | 52 |
| CH2CH3 | L235A, G237A, | TCVVVDVSHEDPEVKFNWYVDGVEVHNAKTKPREEQYNSTYRV | |
| | T366W | VSVLTVLHQDWLNGKEYKCKVSNKALPAPIEKTISKAKGQPRE | |
| | | PQVYTLPPSREEMTKNQVSLWCLVKGFYPSDIAVEWESNGQPE | |
| | | NNYKTTPPVLDSDGSFFLYSKLTVDKSRWQQGNVFSCSVMHEA | |
| | | LHNHYTQKSLSLSPG | |
| CH2CH3 | L234A, L235A, | DKTHTCPPCPAPEAAGGPSVFLFPPKPKDTLMISRTPEVTCVV | 53 |
| | N297G, T366W | VDVSHEDPEVKFNWYVDGVEVHNAKTKPREEQYGSTYRVVSVL | |
| | | TVLHQDWLNGKEYKCKVSNKALPAPIEKTISKAKGQPREPQVY | |
| | | TLPPSREEMTKNQVSLWCLVKGFYPSDIAVEWESNGQPENNYK | |
| | | TTPPVLDSDGSFFLYSKLTVDKSRWQQGNVFSCSVMHEALHNH | |
| | | YTQKSLSLSPG | |
| CH2CH3 | L234A, L235A, | DKTHTCPPCPAPEAAGGPSVFLFPPKPKDTLMISRTPEVTCVV | 54 |
| | N297G, T366S, | VDVSHEDPEVKFNWYVDGVEVHNAKTKPREEQYGSTYRVVSVL | |
| | L368A, Y407V | TVLHQDWLNGKEYKCKVSNKALPAPIEKTISKAKGQPREPQVY | |
| | | TLPPSREEMTKNQVSLSCAVKGFYPSDIAVEWESNGQPENNYK | |
| | | TTPPVLDSDGSFFLVSKLTVDKSRWQQGNVFSCSVMHEALHNH | |
| | | YTQKSLSLSPG | |
| CH2CH3 | L234A, L235A, | DKTHTCPPCPAPEAAGAPSVFLFPPKPKDTLMISRTPEVTCVV | 55 |
| | G237A, T366S, | VDVSHEDPEVKFNWYVDGVEVHNAKTKPREEQYNSTYRVVSVL | |
| | L368A, Y407V | TVLHQDWLNGKEYKCKVSNKALPAPIEKTISKAKGQPREPQVY | |
| | | TLPPSREEMTKNQVSLSCAVKGFYPSDIAVEWESNGQPENNYK | |
| | | TTPPVLDSDGSFFLVSKLTVDKSRWQQGNVFSCSVMHEALHNH | |
| | | YTQKSLSLSPG | |
| CH1CH2C | L234A, L235A, | ASTKGPSVFPLAPSSKSTSGGTAALGCLVKDYFPEPVTVSWNS | 56 |
| H3 | N297G, T366S, | GALTSGVHTFPAVLQSSGLYSLSSVVTVPSSSLGTQTYICNVN | |
| | L368A, Y407V | HKPSNTKVDKRVEPKSCDKTHTCPPCPAPEAAGGPSVFLFPPK | |
| | | PKDTLMISRTPEVTCVVVDVSHEDPEVKFNWYVDGVEVHNAKT | |
| | | KPREEQYGSTYRVVSVLTVLHQDWLNGKEYKCKVSNKALPAPI | |
| | | EKTISKAKGQPREPQVYTLPPSREEMTKNQVSLSCAVKGFYPS | |
| | | DIAVEWESNGQPENNYKTTPPVLDSDGSFFLVSKLTVDKSRWQ | |
| | | QGNVFSCSVMHEALHNHYTQKSLSLSPG | |
| CH1CH2C | L234A, L235A, | ASTKGPSVFPLAPSSKSTSGGTAALGCLVKDYFPEPVTVSWNS | 57 |
| H3 | G237A, T366S, | GALTSGVHTFPAVLQSSGLYSLSSVVTVPSSSLGTQTYICNVN | |
| | L368A, Y407V | HKPSNTKVDKRVEPKSCDKTHTCPPCPAPEAAGAPSVFLFPPK | |
| | | PKDTLMISRTPEVTCVVVDVSHEDPEVKENWYVDGVEVHNAKT | |
| | | KPREEQYNSTYRVVSVLTVLHQDWLNGKEYKCKVSNKALPAPI | |
| | | EKTISKAKGQPREPQVYTLPPSREEMTKNQVSLSCAVKGFYPS | |
| | | DIAVEWESNGQPENNYKTTPPVLDSDGSFFLVSKLTVDKSRWQ | |
| | | QGNVFSCSVMHEALHNHYTQKSLSLSPG | |
| C220S- | C220S, L234A, | EPKSSDKTHTCPPCPAPEAAGAPSVFLFPPKPKDTLMISRTPE | 58 |
| CH2CH3 | L235A, G237A | VTCVVVDVSHEDPEVKFNWYVDGVEVHNAKTKPREEQYNSTYR | |
| | T366W | VVSVLTVLHQDWLNGKEYKCKVSNKALPAPIEKTISKAKGQPR | |
| | | EPQVYTLPPSREEMTKNQVSLWCLVKGFYPSDIAVEWESNGQP | |
| | | ENNYKTTPPVLDSDGSFFLYSKLTVDKSRWQQGNVFSCSVMHE | |
| | | ALHNHYTQKSLSLSPG | |
| CL | | RTVAAPSVFIFPPSDEQLKSGTASVVCLLNNFYPREAKVQWKV | 71 |
| | | DNALQSGNSQESVTEQDSKDSTYSLSSTLTLSKADYEKHKVYA | |
| | | CEVTHQGLSSPVTKSENRGEC | |
| CL | | GQPKAAPSVTLFPPSSEELQANKATLVCLISDFYPGAVTVAWK | 72 |
| | | ADSSPVKAGVETTTPSKQSNNKYAASSYLSLTPEQWKSHRSYS | |
| | | COVTHEGSTVEKTVAPTECS | |
| C220S- | C220S, L234A, | EPKSSDKTHTCPPCPAPEAAGAPSVFLFPPKPKDTLMISRTPE | 120 |
| CH2CH3 + | L235A, G237A | VTCVVVDVSHEDPEVKFNWYVDGVEVHNAKTKPREEQYNSTYR | |
| C-terminal | T366W | VVSVLIVLHQDWLNGKEYKCKVSNKALPAPIEKTISKAKGQPR | |
| Lysine | | EPQVYTLPPSREEMTKNQVSLWCLVKGFYPSDIAVEWESNGQP | |
| | | ENNYKTTPPVLDSDGSFFLYSKLTVDKSRWQQGNVFSCSVMHE | |
| | | ALHNHYTQKSLSLSPGK | |
| CH1CH2C | L234A, L235A, | ASTKGPSVFPLAPSSKSTSGGTAALGCLVKDYFPEPVTVSWNS | 121 |
| H3 with | G237A, T366S, | GALTSGVHTFPAVLQSSGLYSLSSVVTVPSSSLGTQTYICNVN | |
| C-terminal | L368A, Y407V | HKPSNTKVDKRVEPKSCDKTHTCPPCPAPEAAGAPSVFLFPPK | |
| lysine | | PKDTLMISRTPEVTCVVVDVSHEDPEVKFNWYVDGVEVHNAKT | |
| | | KPREEQYNSTYRVVSVLIVLHQDWLNGKEYKCKVSNKALPAPI | |
| | | EKTISKAKGQPREPQVYTLPPSREEMTKNQVSLSCAVKGFYPS | |
| | | DIAVEWESNGQPENNYKTTPPVLDSDGSFFLVSKLTVDKSRWQ | |
| | | QGNVFSCSVMHEALHNHYTQKSLSLSPGK | |

Substitutions are according to EU numbering

TABLE C
Linkers

| | Linker | SEQ ID |
|---|---|---|
| Linker sequence | length (AA) | NO: |
| GSGGGGS | 7 | 81 |
| GGGGSGS | 7 | 82 |
| GSGGGGSGGGGS | 12 | 83 |
| GGGGSGGGGSGS | 12 | 84 |
| GGGGGGGGSGGGGSGGGGS | 20 | 85 |
| GSGGGGSGGGGSGGGGSGGGGS | 22 | 86 |
| GGGGSGGGGSGGGGSGGGGSGS | 22 | 87 |

It is appreciated that CDRs can be modified to include those having one, two or three amino acid additions, deletions and/or substitutions. In some embodiments, the substitutions can be conservative substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide can be replaced with another amino acid residue from the same side chain family. In another embodiment, a string of amino acids can be replaced with a structurally similar string that differs in order and/or composition of side chain family members.

Non-limiting examples of conservative amino acid substitutions are provided in the table below, where a similarity score of 0 or higher indicates conservative substitution between the two amino acids.

TABLE D
Amino Acid Similarity Matrix

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

TABLE E
Conservative Amino Acid Substitutions

| For Amino Acid | Substitution With |
|---|---|
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, D-Gln |
| Aspartic Acid | D-Asp, D-Asn, Asn, Glu, D-Glu, Gln, D-Gln |
| Cysteine | D-Cys, S-Me-Cys, Met, D-Met, Thr, D-Thr, |
| | L-Ser, D-Ser |
| Glutamine | D-Gln, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, β-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me-Cys, Ile, D-Ile, Leu, D-Leu, |
| | Val, D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, Met, D-Met, |
| | Val, D-Val |
| Tyrosine | D-Tyr, Phe, D-Phe, His, D-His, Trp, D-Trp |
| Valine | D-Val, Leu, D-Leu, Ile, D-Ile, Met, D-Met |

It will also be understood by one of ordinary skill in the art that antibody moieties as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence.

According to specific embodiments, the antibody is a humanized antibody. Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321: 522-525 (1986); Riechmann et al., Nature, 332: 323-329 (1988); and Presta, Curr. Op. Struct. Biol., 2: 593-596 (1992)).

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (Jones et al., Nature, 321: 522-525 (1986); Riechmann et al., Nature 332: 323-327 (1988); Verhoeyen et al., Science, 239: 1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The term “multispecific antibody” as used herein refers to an antibody having antigen-binding domains capable of binding to two or more different epitopes within one molecule or is capable of binding to epitopes on two different molecules. Exemplary multispecific antibodies may bind CD3 and one or more tumor antigens or immune cell markers.

Techniques for making multispecific antibodies include, but are not limited to, recombinant co-expression of two or more immunoglobulin heavy chain-light chain pairs having different specificities (see Milstein and Cuello, Nature 305: 537 (1983), WO 93/08829, and Traunecker et al, EMBO J. 10: 3655 (1991)), and “knob-in-hole” engineering (see, e.g., U.S. Pat. No. 5,731,168, WO2009/089004, US2009/0182127, US2011/0287009, Marvin and Zhu, Acta Pharmacol. Sin. (2005) 26(6):649-658, and Kontermann (2005) Acta Pharmacol. Sin., 26: 1-9).

### Constant Regions/Fc Fragments

The multispecific antibodies of the present disclosure, in some embodiments, include a CH1/CL pair within the anti-CD3 Fab and a Fc fragment, along with one or both of the hinge regions. An example sequence of the CL is provided in SEQ ID NO:71. Example CH1-CH3 sequences are included in SEQ ID NO:56, 57, and 121, without limitation.

The Fc region can be engineered to promote heterodimer formulation and/or enhance or eliminate effector function. IgG antibodies can induce direct anti-tumor effects by way of indirect cell killing effects via the Fc-mediated effector functions that engage other immune cells or killer mechanisms. “Effector functions” or “antibody effector functions” as used herein refer to biological activities attributable to the binding of Fc region of an antibody to its effectors such as C1 complex and Fc receptor (FcγRIIa or FcγRIIIa). Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and antibody dependent cell mediated phagocytosis (ADCP), where nonspecific cytotoxic cells that express Fcγ receptors (FcγRs) recognize bound antibody on a target cell and subsequently cause phagocytosis of the target cell.

Among the four IgG subclasses, IgG1 and IgG3 induce the strongest Fc-effector functions. However, since IgG1 has the longest half-life and is more stable than IgG3, most therapeutic antibodies with Fc-mediated functions are of IgG1 isotype.

IgG2 and IgG4 isotypes have significantly lower binding affinity to FcγRs. Recent evidence suggests that the IgG2 isotype is not completely devoid of effector function, whereas the IgG4 isotype can undergo in vivo Fab arm exchange leading to multispecific antibody and off-target effects.

In certain embodiments, the antibody or antigen-binding fragment thereof provided herein is of an isotype of IgG1, IgG2, IgG3 or IgG4. In certain embodiments, the antibody or antigen-binding fragment thereof is of an isotype of IgG1. In certain embodiments, the antibody or antigen-binding fragment thereof is of human IgG1. In certain embodiments, the antibody or antigen-binding fragment thereof is IgG1 with modified Fc domain that have reduced or eliminated effector function.

In certain embodiments, the Fc domain includes reduced ADCC and/or CDC effector function, such as point mutations at L234A and L235A, and optionally G237A (EU index as in Kabat).

In certain embodiments, the Fc domain includes an N297G or N297A or modification (EU index as in Kabat). The Fc region is glycosylated and the carbohydrate can be eliminated by mutation of N297, resulting in a reduction in binding to Fc receptors and C1q.

In certain embodiments, the Fc domain includes mutations of L234A/L235A/N297G, L234A/L235A/N297A, L234A/L235A/G237A, or L234A/L235A/P329G (EU Numbering).

In certain embodiments, the Fc domain described herein is a dimeric Fc region. Usually, two identical Fc regions form a homodimer. However, two different Fc regions can form a heterodimer with mutations to the one or two individual chain via, for example, a knob-into-hole (KIH), a disulfide bond (—S—S—), or via hydrophobic interaction, electrostatic interaction, hydrophilic interaction, or increased flexibility. The Fc region pairing, can be achieved by forming a heterodimer via knob-into-hole (KIH), hydrophobic interaction, electrostatic interaction, hydrophilic interaction, or increased flexibility. For example, the KIH mutations may include T366W (knob) and T366S/L368A/Y407V (hole), S354C/T366W (knob) and Y349C/T366S/L368A/Y407V (hole), T366Y (knob) and Y407T (hole), F405A (knob) and T394W (hole), T366Y/F405A (knob) and T394W/Y407T (hole), or T366W (knob) and Y407A (hole) (EU index as in Kabat).

In certain embodiments, the modified IgG includes mutations to reduce effector function and knob-into-hole (KIH) mutations.

In certain embodiments, the modified IgG includes mutations of L234A/L235A/N297G/T366W in one chain, and L234A/L235A/N297G/T366S/L368A/Y407V in the other chain (EU Numbering).

In certain embodiments, the modified IgG includes mutations of L234A/L235A/G237A/T366W in one chain, and L234A/L235A/G237A/T366S/L368A/Y407V in the other chain (EU Numbering).

The amino acid sequences of IgG constant regions/Fc domains are illustrated in Table B.

In some embodiments, the CH1, CH2, and CH3 of the first chain, collectively, comprise the amino acid sequence of SEQ ID NO:57. In some embodiments, the CH1, CH2, and CH3 of the first chain, collectively, comprise the amino acid sequence of SEQ ID NO:56. In some embodiments, the CH1, CH2, and CH3 of the first chain, collectively, comprise the amino acid sequence of SEQ ID NO:121.

In some embodiments, the CL comprises the amino acid sequence of SEQ ID NO:71.

In some embodiments, the CH2 and CH3 of the third chain, collectively, comprise the amino acid sequence of SEQ ID NO:58. In some embodiments, the CH2 and CH3 of the third chain, collectively, comprise the amino acid sequence of SEQ ID NO:52. In some embodiments, the CH2 and CH3 of the third chain, collectively, comprise the amino acid sequence of SEQ ID NO:120.

In some embodiments, the first chain comprises the amino acid sequence of SEQ ID NO:118 or an amino acid sequence having at least 90%, 91%, 92%, 93%, 94%, 95%, 96%, 97%, 98%, or 99% sequence identity to SEQ ID NO:118. In some embodiments, the first chain comprises the amino acid sequence of SEQ ID NO:123 or an amino acid sequence having at least 90%, 91%, 92%, 93%, 94%, 95%, 96%, 97%, 98%, or 99% sequence identity to SEQ ID NO:123. In some embodiments, the first chain comprises the amino acid sequence of SEQ ID NO:101 or an amino acid sequence having at least 90%, 91%, 92%, 93%, 94%, 95%, 96%, 97%, 98%, or 99% sequence identity to SEQ ID NO:101. In some embodiments, the first chain comprises the amino acid sequence of SEQ ID NO:104 or an amino acid sequence having at least 90%, 91%, 92%, 93%, 94%, 95%, 96%, 97%, 98%, or 99% sequence identity to SEQ ID NO:104. In some embodiments, the first chain comprises the amino acid sequence of SEQ ID NO:107 or an amino acid sequence having at least 90%, 91%, 92%, 93%, 94%, 95%, 96%, 97%, 98%, or 99% sequence identity to SEQ ID NO:107. In some embodiments, the first chain comprises the amino acid sequence of SEQ ID NO:110 or an amino acid sequence having at least 90%, 91%, 92%, 93%, 94%, 95%, 96%, 97%, 98%, or 99% sequence identity to SEQ ID NO:110. In some embodiments, the first chain comprises the amino acid sequence of SEQ ID NO:115 or an amino acid sequence having at least 90%, 91%, 92%, 93%, 94%, 95%, 96%, 97%, 98%, or 99% sequence identity to SEQ ID NO:115.

In some embodiments, the second chain comprises the amino acid sequence of SEQ ID NO:119 or an amino acid sequence having at least 90%, 91%, 92%, 93%, 94%, 95%, 96%, 97%, 98%, or 99% sequence identity to SEQ ID NO:119. In some embodiments, the second chain comprises the amino acid sequence of SEQ ID NO:102 or an amino acid sequence having at least 90%, 91%, 92%, 93%, 94%, 95%, 96%, 97%, 98%, or 99% sequence identity to SEQ ID NO:102. In some embodiments, the second chain comprises the amino acid sequence of SEQ ID NO:105 or an amino acid sequence having at least 90%, 91%, 92%, 93%, 94%, 95%, 96%, 97%, 98%, or 99% sequence identity to SEQ ID NO:105. In some embodiments, the second chain comprises the amino acid sequence of SEQ ID NO:108 or an amino acid sequence having at least 90%, 91%, 92%, 93%, 94%, 95%, 96%, 97%, 98%, or 99% sequence identity to SEQ ID NO:108. In some embodiments, the second chain comprises the amino acid sequence of SEQ ID NO:112 or an amino acid sequence having at least 90%, 91%, 92%, 93%, 94%, 95%, 96%, 97%, 98%, or 99% sequence identity to SEQ ID NO:112. In some embodiments, the second chain comprises the amino acid sequence of SEQ ID NO:113 or an amino acid sequence having at least 90%, 91%, 92%, 93%, 94%, 95%, 96%, 97%, 98%, or 99% sequence identity to SEQ ID NO:113. In some embodiments, the second chain comprises the amino acid sequence of SEQ ID NO:116 or an amino acid sequence having at least 90%, 91%, 92%, 93%, 94%, 95%, 96%, 97%, 98%, or 99% sequence identity to SEQ ID NO:116.

In some embodiments, the third chain comprises the amino acid sequence of SEQ ID NO:117 or an amino acid sequence having at least 90%, 91%, 92%, 93%, 94%, 95%, 96%, 97%, 98%, or 99% sequence identity to SEQ ID NO:117. In some embodiments, the third chain comprises the amino acid sequence of SEQ ID NO:122 or an amino acid sequence having at least 90%, 91%, 92%, 93%, 94%, 95%, 96%, 97%, 98%, or 99% sequence identity to SEQ ID NO:122. In some embodiments, the third chain comprises the amino acid sequence of SEQ ID NO:103 or an amino acid sequence having at least 90%, 91%, 92%, 93%, 94%, 95%, 96%, 97%, 98%, or 99% sequence identity to SEQ ID NO:103. In some embodiments, the third chain comprises the amino acid sequence of SEQ ID NO:106 or an amino acid sequence having at least 90%, 91%, 92%, 93%, 94%, 95%, 96%, 97%, 98%, or 99% sequence identity to SEQ ID NO:106. In some embodiments, the third chain comprises the amino acid sequence of SEQ ID NO:109 or an amino acid sequence having at least 90%, 91%, 92%, 93%, 94%, 95%, 96%, 97%, 98%, or 99% sequence identity to SEQ ID NO:109. In some embodiments, the third chain comprises the amino acid sequence of SEQ ID NO:111 or an amino acid sequence having at least 90%, 91%, 92%, 93%, 94%, 95%, 96%, 97%, 98%, or 99% sequence identity to SEQ ID NO:111. In some embodiments, the third chain comprises the amino acid sequence of SEQ ID NO:114 or an amino acid sequence having at least 90%, 91%, 92%, 93%, 94%, 95%, 96%, 97%, 98%, or 99% sequence identity to SEQ ID NO:114.

In some embodiments, the first chain comprises the amino acid sequence of SEQ ID NO:118 or an amino acid sequence having at least 90%, 91%, 92%, 93%, 94%, 95%, 96%, 97%, 98%, or 99% sequence identity to SEQ ID NO:118. In some embodiments, the second chain comprises the amino acid sequence of SEQ ID NO:119 or an amino acid sequence having at least 90%, 91%, 92%, 93%, 94%, 95%, 96%, 97%, 98%, or 99% sequence identity to SEQ ID NO:119. In some embodiments, the third chain comprises the amino acid sequence of SEQ ID NO:117 or an amino acid sequence having at least 90%, 91%, 92%, 93%, 94%, 95%, 96%, 97%, 98%, or 99% sequence identity to SEQ ID NO:117.

In some embodiments, the first chain, the second chain, and the third chain comprise, respectively, the amino acid sequences of SEQ ID NO:118, 119 and 117.

In some embodiments, the first chain comprises the amino acid sequence of SEQ ID NO:118 or an amino acid sequence having at least 90%, 91%, 92%, 93%, 94%, 95%, 96%, 97%, 98%, or 99% sequence identity to SEQ ID NO:123. In some embodiments, the second chain comprises the amino acid sequence of SEQ ID NO:119 or an amino acid sequence having at least 90%, 91%, 92%, 93%, 94%, 95%, 96%, 97%, 98%, or 99% sequence identity to SEQ ID NO:119. In some embodiments, the third chain comprises the amino acid sequence of SEQ ID NO:117 or an amino acid sequence having at least 90%, 91%, 92%, 93%, 94%, 95%, 96%, 97%, 98%, or 99% sequence identity to SEQ ID NO:122.

In some embodiments, the first chain, the second chain, and the third chain comprise, respectively, the amino acid sequences of SEQ ID NO:123, 119 and 122.

In certain embodiments, the antibody comprises an amino acid sequence or one or more moieties not normally associated with an antibody. Exemplary modifications are described in more detail below. For example, an antibody of the disclosure may comprise a flexible linker sequence, or may be modified to add a functional moiety (e.g., PEG, a drug, a toxin, or a label).

Antibodies, variants, or derivatives thereof of the disclosure include derivatives that are modified, i.e., by the covalent attachment of any type of molecule to the antibody such that covalent attachment does not prevent the antibody from binding to the epitope. For example, but not by way of limitation, the antibodies can be modified, e.g., by glycosylation, acetylation, pegylation, phosphorylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the antibodies may contain one or more non-classical amino acids.

The antibodies can be detectably labeled by coupling it to a chemiluminescent compound. The presence of the chemiluminescent-tagged antigen-binding polypeptide is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

The antibodies can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA). Techniques for conjugating various moieties to an antibody are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al., (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), and Academic Press pp. 303-16 (1985).

In some embodiments, a multispecific antibody of the present disclosure includes three peptide chains that include the amino acid sequences of SEQ ID NOs:115, 116, and 114, respectively. In some embodiments, a multispecific antibody of the present disclosure includes three peptide chains that include the amino acid sequences of SEQ ID NOs: 118, 119, and 117, respectively.

### Polynucleotides Encoding the Antibodies and Methods of Preparing the Antibodies

The present disclosure also provides isolated polynucleotides or nucleic acid molecules encoding the antibodies, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

The present disclosure also provides nucleic acids encoding the antibody polypeptides, recombinant expression vectors and host cells for making the polypeptides, and pharmaceutical compositions comprising the antibody polypeptides disclosed herein. Methods of using the antibody polypeptides of the present disclosure to treat diseases are also provided.

Polynucleotides encoding the polypeptides disclosed herein are typically inserted in an expression vector for introduction into host cells that may be used to produce the desired quantity of the described polypeptides such as antibodies, fragments thereof, or immunoadhesins, i.e., antibody-like protein which combines the binding domain of a heterologous “adhesion” protein (i.e, receptor, ligand or enzyme) with a fragment of immunoglobulin constant domain (i.e., an Fc region). Accordingly, in certain aspects, the invention provides expression vectors comprising polynucleotides disclosed herein and host cells comprising these vectors and polynucleotides.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

Such vectors may easily be chosen from plasmids, phages, and viruses (e.g., baculoviruses, vaccinia viruses, lentiviruses, adenoviruses, adeno-associated viruses, and retroviruses). In general, a vector will comprise a selection marker and appropriate restriction sites to facilitate cloning of the desired gene and the ability of the vector to enter and/or replicate in eukaryotic or prokaryotic cells.

Numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals. In some embodiments the cloned variable region genes are inserted into an expression vector along with the heavy and light chain constant region genes (such as human genes) synthesized as discussed above.

In other embodiments, a binding polypeptide as described herein may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980.

More generally, once a vector or DNA sequence encoding an antibody polypeptide of the present disclosure has been prepared, the expression vector may be introduced into an appropriate host cell. Introduction of the plasmid into the host cell can be accomplished by various techniques such as transfection (including electrophoresis and electroporation), protoplast fusion, calcium phosphate precipitation, cell fusion with enveloped DNA, microinjection, and infection with intact virus. The transformed cells are grown under conditions appropriate to the production of the present binding polypeptides, and assayed for their synthesis. Exemplary assay techniques include enzyme-linked immunosorbent assay (ELISA), radioimmunoassay (RIA), or fluorescence-activated cell sorter analysis (FACS), immunohistochemistry and the like.

Host cells used for expression of the present polypeptide can be of eukaryotic (e.g., mammalian, insect, yeast or plant) or prokaryotic origin. In some embodiments, the host cell line used for expression of the polypeptide is of mammalian origin; those skilled in the art can determine particular host cell lines which are best suited for the desired gene product to be expressed therein. Exemplary host cell lines include, but are not limited to, Chinese hamster ovary (CHO) cells (e.g., DG44 and DUXB11, CHO lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), and 293 (human kidney). In one embodiment, the cell line provides for altered glycosylation, e.g., afucosylation, of the binding polypeptides (e.g., antibodies) expressed therefrom (e.g., PER.C6® (Crucell) or FUT8-knock-out CHO cell lines (POTELLIGENT™ cells) (Biowa, Princeton, N.J.)). In one embodiment, NS0 cells may be used. Host cell lines are typically available from commercial services, the American Tissue Culture Collection, or from published literature. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example, gel filtration, ion-exchange chromatography, chromatography over DEAE-cellulose and/or (immuno-) affinity chromatography.

### Treatments

As shown in the accompanying examples, multispecific antibodies of format Ab101 exhibited reduced activity in inducing T cell activation while retaining potent activity of the attached VHHs. In particular, when the VHHs target immune cells (e.g., B cells), these antibodies are efficient in immune/B cell killing. Such antibodies, therefore, can effectively and safely deplete immune/B cells in a patient which is useful for treating immune/B cell-mediated diseases such as leukemia, lymphoma, and autoimmune diseases.

Accordingly, in some embodiments, the present disclosure provides antibody-based therapies which involve administering the antibodies of the disclosure to a patient such as an animal, a mammal, and a human for treating one or more of the disorders or conditions described herein. Therapeutic compounds of the disclosure include, but are not limited to, antibodies of the disclosure (including variants and derivatives thereof as described herein) and nucleic acids or polynucleotides encoding antibodies of the disclosure (including variants and derivatives thereof as described herein).

The antibodies of the disclosure can also be used to treat or inhibit cancer. In some embodiments, the cancer cells in the patient express or overexpress CD19 and/or BCMA.

Accordingly, in some embodiments, provided are methods for treating a cancer in a patient in need thereof. The method, in one embodiment, entails administering to the patient an effective amount of an antibody of the present disclosure.

Non-limiting examples of cancers include bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer, and thyroid cancer.

In some embodiments, at least one of the cancer cells in the patient over-express CD19. In some embodiments, at least one of the cancer cells in the patient over-express BCMA.

In some embodiments, the cancer is leukemia. In some embodiments, the cancer is lymphoma. In various embodiments, the cancer is acute lymphoblastic leukemia (ALL) (including non T cell ALL), acute myeloid leukemia, B cell prolymphocytic leukemia, B cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia, chronic or acute leukemia, diffuse large B cell lymphoma (DLBCL), follicular lymphoma (FL), hairy cell leukemia, Hodgkin's Disease, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome, non-Hodgkin's lymphoma (NHL), plasma cell proliferative disorder (including asymptomatic myeloma (smoldering multiple myeloma or indolent myeloma), plasmablastic lymphoma, plasmacytoid dendritic cell neoplasm, plasmacytomas (including plasma cell dyscrasia; solitary myeloma; solitary plasmacytoma; extramedullary plasmacytoma; and multiple plasmacytoma), POEMS syndrome (also known as Crow-Fukase syndrome; Takatsuki disease; and PEP syndrome), primary mediastinal large B cell lymphoma (PMBC), small cell- or a large cell-follicular lymphoma, splenic marginal zone lymphoma (SMZL), systemic amyloid light chain amyloidosis, T cell acute lymphoid leukemia (“TALL”), T cell lymphoma, transformed follicular lymphoma, or Waldenstrom macroglobulinemia, Mantle cell lymphoma (MCL), Transformed follicular lymphoma (TFL), Primary mediastinal B cell lymphoma (PMBCL), multiple myeloma, Hairy cell lymphoma/leukemia, or a combination thereof.

The disclosed antibodies and polypeptides can also be used for treating autoimmune diseases. In some embodiments, provided are methods for treating an autoimmune disease or condition in a patient in need thereof. The method, in one embodiment, entails administering to the patient an effective amount of an antibody, polypeptide or polynucleotide the present disclosure.

Examples of neurological (CNS, PNS, NMJ) autoimmune diseases (AID) include, without limitation, multiple sclerosis (MS), neuromyelitis optica spectrum disorder (NMOSD), NMDAR encephalitis, CIPD (Chronic Inflammatory Demyelinating Polyneurophathy) and myasthenia gravis. Examples of AID in rheumatology include, without limitation, rheumatoid arthritis, scleroderma/systemic sclerosis, IIM (idiopathic inflammatory myopathy), and SLE (Lupus nephritis). Examples of AID in dermatology include, without limitation, pemphigus vulgaris, and hidradenitis suppurativa. Examples of AID in hematology include, without limitation, immune thrombocytopenic purpura, antiphospholipid syndrome, and autoimmune hemolytic anemia.

In some embodiments, the autoimmune disease or condition to be treated by the disclosed antibodies, fragments and compositions includes one or more of alopecia areata, autoimmune hemolytic anemia, autoimmune hepatitis, dermatomyositis, diabetes (type 1), celiac disease, autoimmune juvenile idiopathic arthritis, glomerulonephritis, Graves' disease, Guillain-Barré syndrome, idiopathic thrombocytopenic purpura, myasthenia gravis, autoimmune myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, autoimmune thyroiditis, Hashimoto's thyroiditis, autoimmune uveitis, vitiligo, and granulomatosis with polyangiitis (Wegener's).

Examples of neurological (CNS, PNS, NMJ) autoimmune diseases (AID) include, without limitation, multiple sclerosis (MS), neuromyelitis optica spectrum disorder (NMOSD), NMDAR encephalitis, CIPD (Chronic Inflammatory Demyelinating Polyneurophathy), and myasthenia gravis.

Examples of AID in rheumatology include, without limitation, lupus nephritis (LN), non-renal systemic lupus erythematosus (SLE), systemic sclerosis (SSc), idiopathic inflammatory myopathies (IIM), Anti-neutrophil cytoplasmic antibody-associated (ANCA) vasculitis, Sjögren's syndrome, rheumatoid arthritis (RA), and pediatric lupus.

In some embodiments, the autoimmune disease or condition to be treated by the disclosed antibodies, fragments and compositions includes one or more of alopecia areata, autoimmune hemolytic anemia, autoimmune hepatitis, dermatomyositis, diabetes (type 1), celiac disease, autoimmune juvenile idiopathic arthritis, glomerulonephritis, Graves' disease, Guillain-Barré syndrome, idiopathic thrombocytopenic purpura, myasthenia gravis, autoimmune myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, autoimmune thyroiditis, Hashimoto's thyroiditis, autoimmune uveitis, vitiligo, and granulomatosis with polyangiitis (Wegener's).

In some embodiments, autoimmune disease or condition to be treated is relapsed/refractory or moderate to severe, progressive ones. Non-limiting examples include relapsed/refractory or moderate to severe, progressive systemic lupus erythematosus (SLE) with or without lupus nephritis (LN), systemic sclerosis (SSc), or idiopathic inflammatory myopathy (IIM).

Systemic lupus erythematosus (SLE) is a chronic, multisystem autoimmune disease of unknown cause, predominantly affecting women of childbearing age. The disease manifests in a wide array of symptoms, including general constitutional complaints, skin rashes, serositis, inflammatory arthritis, kidney disease, systemic vasculitis, lymph node enlargement, splenomegaly, hemolytic anemia, cognitive impairment, and central nervous system involvement. SLE is a severe and potentially life-threatening condition that can impact any organ system. It poses a significant morbidity and mortality while profoundly affecting patients' daily functioning and quality of life. SLE is associated with increased morbidity and mortality, with an estimated 10% to 15% of patients facing premature death due to lupus-related complications.

Persistent, uncontrolled disease activity over time leads to poorer outcomes, including organ damage and coronary artery disease. Approximately 50% to 60% of SLE patients develop lupus nephritis (LN) within the first 3 years of diagnosis, which significantly increases the risk of renal failure and death. The toxicities associated with both approved and off-label treatments for SLE contribute to the long-term morbidity and mortality observed in these patients.

Systemic sclerosis (SSc), also known as scleroderma, is a rare autoimmune connective tissue disease characterized by immune-mediated fibrosis, vasculopathy, and significant systemic involvement, leading to substantial morbidity and mortality. Common features include skin thickening, Raynaud's phenomenon, and digital ulcers. Systemic sclerosis is further categorized into ‘limited’ and ‘diffuse cutaneous’ SSc based on the extent of the skin involvement, with each having distinctive clinical and serologic features. However, the systemic nature of SSc is underscored by frequent extra-cutaneous manifestations, such as interstitial lung disease (ILD), pulmonary arterial hypertension (PAH), renal crisis, and gastrointestinal involvement. Diffuse cutaneous systemic sclerosis (dcSSc) is associated with high mortality resulting from early internal-organ involvement.

The estimated incidence of SSc ranges from 1 to 2 per 100,000 person-years, with a prevalence of 7 to 50 per 100,000 people, depending on the population studied. In a systematic review and meta-analysis analyzing the global incidence and prevalence of SSc, the pooled incidence rate of SSc was 1.4 per 100,000 person-years and the prevalence rate was 17.6 cases per 100,000 people. Regional epidemiological studies estimated higher numbers, with prevalence up to 44.3 per 100,000 people in North America. SSc is more common in women than in men, with a peak onset between the ages of 30 and 50. The prognosis for SSc varies widely based on the extent of internal organ involvement. ILD, in particular, is a significant predictor of mortality, and patients with ILD experience worse outcomes compared to those without lung involvement.

Idiopathic inflammatory myopathy (IIM), commonly referred to as myositis, are a group of rare autoimmune diseases primarily characterized by immune-mediated muscle injury and significant systemic involvement, leading to substantial morbidity and mortality. Common features include muscle weakness, low muscle endurance, and myalgia. However, the systemic nature of the disease is underscored by frequent extramuscular manifestations such as skin rashes, arthritis, ILD, and heart involvement.

The Bohan and Peter 1975 criteria and the ACR/European league against rheumatism (EULAR) 2017 criteria are both used in the diagnosis and classification of IIM. The criteria recognize 5 main types of IIM: dermatomyositis (DM), immune-mediated necrotizing myopathy (IMNM), anti-synthetase syndrome, inclusion body myositis (IBM), and polymyositis (PM), each distinguished by characteristic clinical, serologic, and pathological features. Many patients previously classified as having PM per the Bohan and Peter 1975 criteria are now reclassified into these subgroups, resulting in a decline in the number of patients considered to have PM.

Data indicate that PM, DM, and IMNM are more common in women, while IBM is more prevalent in men. The incidence of IIM increases with age, peaking around 50 years of age. The estimated incidence of IIM ranges from 0.2 to 2 per 100,000 person-years, with a prevalence of 2 to 25 per 100,000 people. However, these estimates vary widely depending on factors such as age, gender, geographical location, ethnicity, and study methodology. Mortality rates also show considerable variation, with 10-year survival rates ranging from 20% to 90% in studies from Europe, North America, and Japan. ILD is a significant prognostic factor in IIM, with mortality rates much higher in those with ILD compared to those without. Additionally, there is a strong association between adult-onset IIM and malignancy, particularly in patients with DM.

Rheumatoid arthritis (RA) is a long-term autoimmune disorder that primarily affects joints. It typically results in warm, swollen, and painful joints. Pain and stiffness often worsen following rest. Most commonly, the wrist and hands are involved, with the same joints typically involved on both sides of the body. The disease may also affect other parts of the body. While the cause of rheumatoid arthritis is not clear, it is believed to involve a combination of genetic and environmental factors. The underlying mechanism involves the body's immune system attacking the joints. This results in inflammation and thickening of the joint capsule. The goals of treatment are to reduce pain, decrease inflammation, and improve a person's overall functioning. Pain medications, steroids, and NSAIDs are frequently used to help with symptoms. A group of medications called disease-modifying antirheumatic drugs (DMARDs), such as hydroxychloroquine and methotrexate, may be used to try to slow the progression of disease.

Osteoarthritis (OA) is a type of joint disease that results from breakdown of joint cartilage and underlying bone. The most common symptoms are joint pain and stiffness. Initially, symptoms may occur only following exercise, but over time may become constant. Other symptoms may include joint swelling, decreased range of motion, and when the back is affected weakness or numbness of the arms and legs. Causes include previous joint injury, abnormal joint or limb development, and inherited factors. Risk is greater in those who are overweight, have one leg of a different length, and have jobs that result in high levels of joint stress. Osteoarthritis is believed to be caused by mechanical stress on the joint and low grade inflammatory processes. Treatment includes exercise, efforts to decrease joint stress, support groups, and pain medications.

Multiple sclerosis (MS) is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. While the cause is not clear, the underlying mechanism is thought to be either destruction by the immune system or failure of the myelin-producing cells. There is no known cure for multiple sclerosis. Treatments attempt to improve function after an attack and prevent new attacks.

Asthma is a common long-term inflammatory disease of the airways of the lungs. It is characterized by variable and recurring symptoms, reversible airflow obstruction, and bronchospasm. Symptoms include episodes of wheezing, coughing, chest tightness, and shortness of breath. Asthma is thought to be caused by a combination of genetic and environmental factors. Environmental factors include exposure to air pollution and allergens. Asthma is classified according to the frequency of symptoms, forced expiratory volume in one second (FEV1), and peak expiratory flow rate. It may also be classified as atopic or non-atopic, where atopy refers to a predisposition toward developing a type 1 hypersensitivity reaction. There is no cure for asthma. Symptoms can be prevented by avoiding triggers, such as allergens and irritants, and by the use of inhaled corticosteroids. Long-acting beta agonists (LABA) or antileukotriene agents may be used in addition to inhaled corticosteroids if asthma symptoms remain uncontrolled. Treatment of rapidly worsening symptoms is usually with an inhaled short-acting beta-2 agonist such as salbutamol and corticosteroids taken by mouth. In very severe cases, intravenous corticosteroids, magnesium sulfate, and hospitalization may be required.

Chronic obstructive pulmonary disease (COPD) is a type of obstructive lung disease characterized by long-term poor airflow. COPD can include two main conditions, emphysema and chronic bronchitis. In emphysema, the walls between many of the air sacs are damaged. As a result, the air sacs lose their shape and become floppy. This damage also can destroy the walls of the air sacs, leading to fewer and larger air sacs instead of many tiny ones. If this happens, the amount of gas exchange in the lungs is reduced. In chronic bronchitis, the lining of the airways stays constantly irritated and inflamed, and this causes the lining to swell. Lots of thick mucus forms in the airways, making it hard to breathe. There is no known cure for COPD, but the symptoms are treatable and its progression can be delayed.

Pain is a distressing feeling often caused by intense or damaging stimuli, such as stubbing a toe, burning a finger, putting alcohol on a cut, or bumping the “funny bone”. Pain is a complex, subjective phenomenon, defining pain has been a challenge. Pain is also referred to as an unpleasant sensory and emotional experience associated with actual or potential tissue damage. Pain is sometimes regarded as a symptom of an underlying condition, such as inflammation.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the antibodies, variants or include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the antigen-binding polypeptides or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. In some embodiments, when administering a protein, including an antibody, of the disclosure, care must be taken to use materials to which the protein does not absorb.

The amount of the antibodies of the disclosure which will be effective in the treatment, inhibition and prevention of an immune or malignant disease, disorder or condition can be determined by standard clinical techniques. In addition, in vitro assays may optionally be employed to help identify optimal dosage ranges. The precise dose to be employed in the formulation will also depend on the route of administration, and the seriousness of the disease, disorder or condition, and should be decided according to the judgment of the practitioner and each patient's circumstances. Effective doses may be extrapolated from dose-response curves derived from in vitro or animal model test systems.

As a general proposition, the dosage administered to a patient of the antigen-binding polypeptides of the present disclosure is typically 0.1 mg/kg to 100 mg/kg of the patient's body weight, between 0.1 mg/kg and 20 mg/kg of the patient's body weight, or 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation.

In an additional embodiment, the compositions of the disclosure are administered in combination with cytokines. Cytokines that may be administered with the compositions of the disclosure include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions of the disclosure are administered in combination with other therapeutic or prophylactic regimens, such as, for example, radiation therapy.

### Compositions

The present disclosure also provides pharmaceutical compositions. Such compositions include an effective amount of an antibody described herein, and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, in some embodiments in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### EXAMPLES

### Example 1: Generation of Multispecific Antibodies

Multi-specific antibodies having specificity to CD3, as well as to CD19 and/or BCMA have been designed and prepared. The molecules included Ab1, Ab25, Ab53, Ab22, Ab27, Ab65 and Ab101 (and Ab101-1) whose structures are illustrated in FIG. 1 and Table 1. The sequences are provided in Table 2.

TABLE 1
Structures of antibodies

| Antibody | Chain 1/Chain 2/Chain 3 | Figure |
|---|---|---|
| Ab1 | A1/B1/C2 | FIG. 1A |
| Ab22 | A6/B2/C9 | FIG. 1B |
| Ab25 | A8/B3/C5 | FIG. 1C |
| Ab27 | A8/B3/C2 | FIG. 1D |
| Ab53 | A28/B24/C27 | FIG. 1E |
| Ab65 | A28/B25/C34 | FIG. 1F |
| Ab101 | A24/B28/C34 | FIG. 1G |
| Ab101-1 | H4/L3/H3 | FIG. 1H |

TABLE 2
Antibody sequences

| | | SEQ ID |
|---|---|---|
| Chain | Amino acid sequence | NO: |
| A1 | EVQLVESGGGLVQPGGSLKLSCAASGFTENTYAMNWVRQASGKGLEWVARIRSKYNNYA | 101 |
| | TYYADSVKDRFTISRDDSLNTLYLQMNSLKTEDTAVYYCVRHGNFGNSYVSWFAYWGQG | |
| | TLVTVSS [CD3 VH (SEQ ID NO: 15)] | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHTCPPCPAPEAA | |
| | GGPSVFLFPPKPKDTLMISRTPEVTCVVVDVSHEDPEVKENWYVDGVEVHNAKTKPREE | |
| | QYGSTYRVVSVLTVLHQDWLNGKEYKCKVSNKALPAPIEKTISKAKGQPREPQVYTLPP | |
| | SREEMTKNQVSLSCAVKGFYPSDIAVEWESNGQPENNYKTTPPVLDSDGSFFLVSKLTV | |
| | DKSRWQQGNVFSCSVMHEALHNHYTQKSLSLSPG [CH1CH2CH3 with L234A, | |
| | L235A, N297G, T366S, L368A, Y407V (SEQ ID NO: 56)] | |
| B1 | QAVVTQEPSLTVSPGGTVTLTCRSSTGAVTTSNYANWVQEKPGQLFRGLIGGINKRAPW | 102 |
| | VPARFSGSLLGDKAALTLSGAQPEDEAEYFCALWYSNLWVFGGGTKLTVL [CD3 VL | |
| | (SEQ ID NO: 19)] | |
| | RTVAAPSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSENRGEC [CL (SEQ | |
| | ID NO: 71)] | |
| C2 | QVQLVESGGGLVQDGDSLWLSCAASGAFFGVGIGWFRQAPGKEREFVAAITWSGGRTWY | 103 |
| | TKDVKGRFIISADKPRNTVLLQMNDLKSEDTAVYYCASGDPSGGAGPESVRQYLHWGQG | |
| | TQVTVSS [CD19 VHH (SEQ ID NO: 1)] | |
| | DKTHTCPPCPAPEAAGGPSVFLFPPKPKDTLMISRTPEVTCVVVDVSHEDPEVKENWYV | |
| | DGVEVHNAKTKPREEQYGSTYRVVSVLTVLHQDWLNGKEYKCKVSNKALPAPIEKTISK | |
| | AKGQPREPQVYTLPPSREEMTKNQVSLWCLVKGFYPSDIAVEWESNGQPENNYKTTPPV | |
| | LDSDGSFFLYSKLTVDKSRWQQGNVFSCSVMHEALHNHYTQKSLSLSPG [CH2H3 | |
| | with L234A, L235A, N297G, T366W (SEQ ID NO: 53)] | |
| A6 | EVQLVESGGGLVQPGGSLKLSCAASGFTENTYAMNWVRQASGKGLEWVARIRSKYNNYA | 104 |
| | TYYADSVKDRFTISRDDSLNTLYLQMNSLKTEDTAVYYCVRHGNFGNSYVSWFAYWGQG | |
| | TLVTVSS [CD3 VH (SEQ ID NO: 15)] | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHTCPPCPAPEAA | |
| | GAPSVFLFPPKPKDTLMISRTPEVTCVVVDVSHEDPEVKENWYVDGVEVHNAKTKPREE | |
| | QYNSTYRVVSVLTVLHQDWLNGKEYKCKVSNKALPAPIEKTISKAKGQPREPQVYTLPP | |
| | SREEMTKNQVSLSCAVKGFYPSDIAVEWESNGQPENNYKTTPPVLDSDGSFFLVSKLTV | |
| | DKSRWQQGNVFSCSVMHEALHNHYTQKSLSLSPG [CH1CH2CH3 with L234A, | |
| | L235A, G237A, T366S, L368A, Y407V (SEQ ID NO: 57)] | |
| B2 | QAVVTQEPSLTVSPGGTVTLTCRSSTGAVTTSNYANWVQQKPGQAFRGLIGGTNKRAPW | 105 |
| | TPARFSGSLLGGKAALTLSGAQPEDEAEYFCALWYSNLWVFGGGTKLTVL [CD3 VL2 | |
| | (SEQ ID NO: 20)] | |
| | RTVAAPSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSENRGEC [CL (SEQ | |
| | ID NO: 71)] | |
| C9 | EVQLVESGGGLVQPGGSLRLSCAASGAFFGVGIGWFRQAPGKGREFVAAITWSGGRTWY | 106 |
| | TKDVKGRFTISADKSKNTVYLQMNSLRAEDTAVYYCASGDPSGGAGPESVRQYLHWGQG | |
| | TLVTVSS [CD19 hVHH (SEQ ID NO: 2)] | |
| | GGGGSGGGGSGGGGSGGGGS [linker (SEQ ID NO: 85)] | |
| | EVQLVESGGGLVQPGGSLRLSCAASGAFFGVGIGWFRQAPGKGREFVAAITWSGGRTWY | |
| | TKDVKGRFTISADKSKNTVYLQMNSLRAEDTAVYYCASGDPSGGAGPESVRQYLHWGQG | |
| | TLVTVSS [CD19 hVHH (SEQ ID NO: 2)] | |
| | DKTHTCPPCPAPEAAGAPSVFLFPPKPKDTLMISRTPEVTCVVVDVSHEDPEVKENWYV | |
| | DGVEVHNAKTKPREEQYNSTYRVVSVLTVLHQDWLNGKEYKCKVSNKALPAPIEKTISK | |
| | AKGQPREPQVYTLPPSREEMTKNQVSLWCLVKGFYPSDIAVEWESNGQPENNYKTTPPV | |
| | LDSDGSFFLYSKLTVDKSRWQQGNVFSCSVMHEALHNHYTQKSLSLSPG [CH2CH3 | |
| | with L234A, L235A, G237A, T366W (SEQ ID NO: 51)] | |
| A8 | QVQLVESGGGLVQPGGSLRLTCVASGSGDNFFSMGWFRQAPGKOREWVAFIYDQRATLY | 107 |
| | RDSVKGRFTISRDSAKNTAFLQMNSLKPEDIGVYYCNVILDSGTGDSDRSTYWGQGTQV | |
| | TVSS [BCMA hVHH3 (SEQ ID NO: 11)] | |
| | GSGGGGSGGGGS [linker (SEQ ID NO: 83)] | |
| | EVQLVESGGGLVQPGGSLKLSCAASGFTFNTYAMNWVRQASGKGLEWVARIRSKYNNYA | |
| | TYYADSVKDRFTISRDDSLNTLYLQMNSLKTEDTAVYYCVRHGNFGNSYVSWFAYWGQG | |
| | TLVTVSS [CD3 VH (SEQ ID NO: 15)] | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHTCPPCPAPEAA | |
| | GAPSVFLFPPKPKDTLMISRTPEVTCVVVDVSHEDPEVKFNWYVDGVEVHNAKTKPREE | |
| | QYNSTYRVVSVLTVLHQDWLNGKEYKCKVSNKALPAPIEKTISKAKGQPREPQVYTLPP | |
| | SREEMTKNQVSLSCAVKGFYPSDIAVEWESNGQPENNYKTTPPVLDSDGSFFLVSKLTV | |
| | DKSRWQQGNVFSCSVMHEALHNHYTQKSLSLSPG [CH1CH2CH3 with L234A, | |
| | L235A, G237A, T366S, L368A, Y407V (SEQ ID NO: 57)] | |
| B3 | QVQLVESGGGLVQDGDSLWLSCAASGAFFGVGIGWFRQAPGKEREFVAAITWSGGRTWY | 108 |
| | TKDVKGRFIISADKPRNTVLLQMNDLKSEDTAVYYCASGDPSGGAGPESVRQYLHWGQG | |
| | TQVTVSS [CD19 VHH (SEQ ID NO: 1)] | |
| | GGGGSGS [linker (SEQ ID NO: 82)] | |
| | QAVVTQEPSLTVSPGGTVTLTCRSSTGAVTTSNYANWVQEKPGQLFRGLIGGINKRAPW | |
| | VPARF SGSLLGDKAALTLSGAQPEDEAEYFCALWYSNLWVFGGGTKLTVL [CD3 VL | |
| | (SEQ ID NO: 19)] | |
| | RTVAAPSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSFNRGEC [CL (SEQ | |
| | ID NO: 71)] | |
| C5 | EPSSDKTHTCPPCPAPEAAGAPSVFLFPPKPKDTLMISRTPEVTCVVVDVSHEDPEVKF | 109 |
| | NWYVDGVEVHNAKTKPREEQYNSTYRVVSVLTVLHQDWLNGKEYKCKVSNKALPAPIEK | |
| | TISKAKGQPREPQVYTLPPSREEMTKNQVSLWCLVKGFYPSDIAVEWESNGQPENNYKT | |
| | TPPVLDSDGSFFLYSKLTVDKSRWQQGNVFSCSVMHEALHNHYTQKSLSLSPG | |
| | [C220S-CH2CH3 with L234A, L235A, G237A, T366W (SEQ ID | |
| | NO: 52)] | |
| A28 | EVQLVESGGGLVQPGGSLRLSCAASGAFFGVGIGWFRQAPGKGREFVAAITWSGGRTWY | 110 |
| | TKDVKGRFTISADKSKNTVYLQMNSLRAEDTAVYYCASGDPSGGAGPESVRQYLHWGQG | |
| | TLVTVSS [CD19 hVHH (SEQ ID NO: 2)] | |
| | GSGGGGSGGGGS [linker (SEQ ID NO: 83)] | |
| | EVQLVESGGGLVQPGGSLKLSCAASGFTFNTYAMNWVRQASGKGLEWVARIRSKYNNYA | |
| | TYYADSVKDRFTISRDDSLNTLYLQMNSLKTEDTAVYYCVRHGNFGNSYVSWFAYWGQG | |
| | TLVTVSS [CD3 VH (SEQ ID NO: 15)] | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHTCPPCPAPEAA | |
| | GAPSVFLFPPKPKDTLMISRTPEVTCVVVDVSHEDPEVKFNWYVDGVEVHNAKTKPREE | |
| | QYNSTYRVVSVLTVLHQDWLNGKEYKCKVSNKALPAPIEKTISKAKGQPREPQVYTLPP | |
| | SREEMTKNQVSLSCAVKGFYPSDIAVEWESNGQPENNYKTTPPVLDSDGSFFLVSKLTV | |
| | DKSRWQQGNVFSCSVMHEALHNHYTOKSLSLSPG [CH1CH2CH3 with L234A, | |
| | L235A, G237A, T366S, L368A, Y407V (SEQ ID NO: 57)] | |
| C27 | EPSSDKTHTCPPCPAPEAAGAPSVFLFPPKPKDTLMISRTPEVTCVVVDVSHEDPEVKF | 111 |
| | NWYVDGVEVHNAKTKPREEQYNSTYRVVSVLTVLHQDWLNGKEYKCKVSNKALPAPIEK | |
| | TISKAKGQPREPQVYTLPPSREEMTKNQVSLWCLVKGFYPSDIAVEWESNGQPENNYKT | |
| | TPPVLDSDGSFFLYSKLTVDKSRWQQGNVFSCSVMHEALHNHYTQKSLSLSPG | |
| | [C220S-CH2CH3 with L234A, L235A, G237A, T366W (SEQ ID | |
| | NO: 52)] | |
| B24 | EVQLVESGGGLVQPGGSLRLSCATSGFTFSPSAMNWVRQAPGKGLEWVADIYGDGRAVY | 112 |
| | ADSVKGRFTVSRDNSKNLVYLQMNSLRAEDTAVYVCGVGRYTTTPLSEKLWGQGTLVTV | |
| | SS [BCMA hVHH1 (SEQ ID NO: 6)] | |
| | GGGGSGGGGSGS [linker (SEQ ID NO: 84)] | |
| | QAVVTQEPSLTVSPGGTVTLTCRSSTGAVTTSNYANWVQEKPGQLFRGLIGGINKRAPW | |
| | VPARFSGSLLGDKAALTLSGAQPEDEAEYFCALWYSNLWVFGGGTKLTVL [CD3 VL | |
| | (SEQ ID NO: 19)] | |
| | RTVAAPSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSENRGEC [CL (SEQ | |
| | ID NO: 71)] | |
| B25 | EVQLVESGGGLVQPGGSLRLSCATSGFTFSPSAMNWVRQAPGEGLEWVADIYGDGRAVY | 113 |
| | ADSVKGRFTVSRDNAKKLVYLEMNDLRPEDTAVYVCGVGRYTTTPLSEKLRGQGTLVTV | |
| | SS [BCMA hVHH2 (SEQ ID NO: 7)] | |
| | GGGGSGGGGSGS [linker (SEQ ID NO: 84)] | |
| | QAVVTQEPSLTVSPGGTVTLTCRSSTGAVTTSNYANWVQEKPGQLFRGLIGGTNKRAPW | |
| | VPARFSGSLLGDKAALTLSGAQPEDEAEYFCALWYSNLWVFGGGTKLTVL [CD3 VL | |
| | (SEQ ID NO: 19)] | |
| | RTVAAPSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSENRGEC [CL (SEQ | |
| | ID NO: 71)] | |
| C34 | EVQLVESGGGLVQPGGSLRLSCAASGAFFGVGIGWFRQAPGKGREFVAAITWSGGRTWY | 114 |
| | TKDVKGRFTISADKSKNTVYLQMNSLRAEDTAVYYCASGDPSGGAGPESVRQYLHWGQG | |
| | TLVTVSS [CD19 hVHH (SEQ ID NO: 2)] | |
| | EPSSDKTHTCPPCPAPEAAGAPSVFLFPPKPKDTLMISRTPEVTCVVVDVSHEDPEVKF | |
| | NWYVDGVEVHNAKTKPREEQYNSTYRVVSVLTVLHQDWLNGKEYKCKVSNKALPAPIEK | |
| | TISKAKGQPREPQVYTLPPSREEMTKNQVSLWCLVKGFYPSDIAVEWESNGQPENNYKT | |
| | TPPVLDSDGSFFLYSKLTVDKSRWQQGNVFSCSVMHEALHNHYTQKSLSLSPG | |
| | [C220S-CH2CH3 with L234A, L235A, G237A, T366W (SEQ ID | |
| | NO: 52)] | |
| A24 | EVQLVESGGGLVQPGGSLRLSCATSGFTFSPSAMNWVRQAPGKGLEWVADIYGDGRAVY | 115 |
| | ADSVKGRFTVSRDNSKNLVYLQMNSLRAEDTAVYVCGVGRYTTTPLSEKLWGQGTLVTV | |
| | SS [BCMA hVHH1 (SEQ ID NO: 6)] | |
| | GSGGGGSGGGGS [linker (SEQ ID NO: 83)] | |
| | EVQLVESGGGLVQPGGSLKLSCAASGFTENTYAMNWVRQASGKGLEWVARIRSKYNNYA | |
| | TYYADSVKDRFTISRDDSLNTLYLQMNSLKTEDTAVYYCVRHGNFGNSYVSWFAYWGQG | |
| | TLVTVSS [CD3 VH (SEQ ID NO: 15)] | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHTCPPCPAPEAA | |
| | GAPSVFLFPPKPKDTLMISRTPEVTCVVVDVSHEDPEVKENWYVDGVEVHNAKTKPREE | |
| | QYNSTYRVVSVLTVLHQDWLNGKEYKCKVSNKALPAPIEKTISKAKGQPREPQVYTLPP | |
| | SREEMTKNQVSLSCAVKGFYPSDIAVEWESNGQPENNYKTTPPVLDSDGSFFLVSKLTV | |
| | DKSRWQQGNVFSCSVMHEALHNHYTQKSLSLSPG [CH1CH2CH3 with L234A, | |
| | L235A, G237A, T366S, L368A, Y407V (SEQ ID NO: 57)] | |
| B28 | EVQLVESGGGLVQPGGSLRLSCAASGAFFGVGIGWFRQAPGKGREFVAAITWSGGRTWY | 116 |
| | TKDVKGRFTISADKSKNTVYLQMNSLRAEDTAVYYCASGDPSGGAGPESVRQYLHWGQG | |
| | TLVTVSS [CD19 hVHH (SEQ ID NO: 2)] | |
| | GGGGSGGGGSGS [linker (SEQ ID NO: 84)] | |
| | QAVVTQEPSLTVSPGGTVTLTCRSSTGAVTTSNYANWVQEKPGQLFRGLIGGTNKRAPW | |
| | VPARF SGSLLGDKAALTLSGAQPEDEAEYFCALWYSNLWVFGGGTKLTVL [CD3 VL | |
| | (SEQ ID NO: 19)] | |
| | RTVAAPSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSENRGEC [CL (SEQ | |
| | ID NO: 71)] | |
| H3 | EVQLVESGGGLVQPGGSLRLSCAASGAFFGVGIGWFRQAPGKGREFVAAITWSGGRTWY | 117 |
| | TKDVKGRFTISADKSKNTVYLQMNSLRAEDTAVYYCASGDPSGGAGPESVRQYLHWGQG | |
| | TLVTVSS [CD19 hVHH (SEQ ID NO: 2)] | |
| | EPKSSDKTHTCPPCPAPEAAGAPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKALPAPIE | |
| | KTISKAKGQPREPQVYTLPPSREEMTKNQVSLWCLVKGFYPSDIAVEWESNGQPENNYK | |
| | TTPPVLDSDGSFFLYSKLTVDKSRWQQGNVFSCSVMHEALHNHYTQKSLSLSPG | |
| | [C220S-CH2CH3 with C220S, L234A, L235A, G237A T366W (SEQ ID | |
| | NO: 58)] | |
| H4 | EVQLVESGGGLVQPGGSLRLSCATSGFTFSPSAMNWVRQAPGKGLEWVADIYGDGRAVY | 118 |
| | ADSVKGRFTVSRDNSKNLVYLQMNSLRAEDTAVYVCGVGRYTTTPLSEKLWGQGTLVTV | |
| | SS [BCMA hVHH1 (SEQ ID NO: 6)] | |
| | GSGGGGSGGGGS [linker (SEQ ID NO: 83)] | |
| | EVQLVESGGGLVQPGGSLKLSCAASGFTFNTYAMNWVRQASGKGLEWVARIRSKYNNYA | |
| | TYYADSVKDRFTISRDDSLNTLYLQMNSLKTEDTAVYYCVRHGNFGNSYVSWFAYWGQG | |
| | TLVTVSS [CD3 VH (SEQ ID NO: 15)] | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHTCPPCPAPEAA | |
| | GAPSVFLFPPKPKDTLMISRTPEVTCVVVDVSHEDPEVKFNWYVDGVEVHNAKTKPREE | |
| | QYNSTYRVVSVLTVLHQDWLNGKEYKCKVSNKALPAPIEKTISKAKGQPREPQVYTLPP | |
| | SREEMTKNQVSLSCAVKGFYPSDIAVEWESNGQPENNYKTTPPVLDSDGSFFLVSKLTV | |
| | DKSRWQQGNVFSCSVMHEALHNHYTQKSLSLSPG [CH1CH2CH3 with L234A, | |
| | L235A, G237A, T366S, L368A, Y407V (SEQ ID NO: 57)] | |
| L3 | EVQLVESGGGLVQPGGSLRLSCAASGAFFGVGIGWFRQAPGKGREFVAAITWSGGRTWY | 119 |
| | TKDVKGRFTISADKSKNTVYLQMNSLRAEDTAVYYCASGDPSGGAGPESVRQYLHWGQG | |
| | TLVTVSS [CD19 hVHH (SEQ ID NO: 2)] | |
| | GGGGGGGGSGS [linker (SEQ ID NO: 84)] | |
| | QTVVTQEPSLTVSPGGTVTLTCRSSTGAVTTSNYANWVQEKPGQLFRGLIGGTNKRAPW | |
| | VPARFSGSLLGDKAALTLSGAQPEDEAEYFCALWYSNLWVFGGGTKLTVL [CD3 VL3 | |
| | (SEQ ID NO: 21)] | |
| | RTVAAPSVFIFPPSDEQLKSGTASVVCLLNNFYPREAKVQWKVDNALQSGNSQESVTEQ | |
| | DSKDSTYSLSSTLTLSKADYEKHKVYACEVTHQGLSSPVTKSENRGEC [CL (SEQ | |
| | ID NO: 71)] | |
| H5 | EVQLVESGGGLVQPGGSLRLSCAASGAFFGVGIGWFRQAPGKGREFVAAITWSGGRTWY | 122 |
| | TKDVKGRFTISADKSKNTVYLQMNSLRAEDTAVYYCASGDPSGGAGPESVRQYLHWGQG | |
| | TLVTVSS [CD19 hVHH (SEQ ID NO: 2)] | |
| | EPKSSDKTHTCPPCPAPEAAGAPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKALPAPIE | |
| | KTISKAKGQPREPQVYTLPPSREEMTKNQVSLWCLVKGFYPSDIAVEWESNGQPENNYK | |
| | TTPPVLDSDGSFFLYSKLTVDKSRWQQGNVFSCSVMHEALHNHYTQKSLSLSPGK | |
| | [C220S-CH2CH3 with C220S, L234A, L235A, G237A T366W (SEQ ID | |
| | NO: 120)] | |
| H6 | EVQLVESGGGLVQPGGSLRLSCATSGFTFSPSAMNWVRQAPGKGLEWVADIYGDGRAVY | 123 |
| | ADSVKGRFTVSRDNSKNLVYLQMNSLRAEDTAVYVCGVGRYTTTPLSEKLWGQGTLVTV | |
| | SS [BCMA hVHH1 (SEQ ID NO: 6)] | |
| | GSGGGGSGGGGS [linker (SEQ ID NO: 83)] | |
| | EVQLVESGGGLVQPGGSLKLSCAASGFTFNTYAMNWVRQASGKGLEWVARIRSKYNNYA | |
| | TYYADSVKDRFTISRDDSLNTLYLQMNSLKTEDTAVYYCVRHGNFGNSYVSWFAYWGQG | |
| | TLVTVSS [CD3 VH (SEQ ID NO: 15)] | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKRVEPKSCDKTHTCPPCPAPEAA | |
| | GAPSVFLFPPKPKDTLMISRTPEVTCVVVDVSHEDPEVKFNWYVDGVEVHNAKTKPREE | |
| | QYNSTYRVVSVLTVLHQDWLNGKEYKCKVSNKALPAPIEKTISKAKGQPREPQVYTLPP | |
| | SREEMTKNQVSLSCAVKGFYPSDIAVEWESNGQPENNYKTTPPVLDSDGSFFLVSKLTV | |
| | DKSRWQQGNVFSCSVMHEALHNHYTQKSLSLSPGK [CH1CH2CH3 with L234A, | |
| | L235A, G237A, T366S, L368A, Y407V (SEQ ID NO: 121)] | |

Each of these multispecific antibodies included a monovalent anti-CD3 core which includes a VH-CH1/VL-CL pair attached to a Fc fragment. The monovalent anti-CD3 core had three N-termini, i.e., the N-terminus of the VH-CH1-CH2-CH3 heavy chain (chain 1), the N-terminus of the VL-CL light chain (chain 2), and the N-terminus of the CH2-CH3 fragment (chain 3). Each of these N-termini potentially served as an anchor point for an anti-CD19 or anti-BCMA VHH (or a chain having two or more VHH).

In Ab1, an anti-CD19 VHH was fused to the N-terminus of chain 3 (FIG. 1A). In Ab22, a fusion of two tandem anti-CD19 VHHs was fused to the N-terminus of chain 3 (FIG. 1B). In Ab25, an anti-BCMA VHH and an anti-CD19 VHH are fused to the N-termini of chains 1 and 2, respectively (FIG. 1C). In Ab53, an anti-CD19 and an anti-BCMA VHH are fused to the N-termini of chains 1 and 2, respectively (FIG. 1E). In Ab27 (FIG. 1C), Ab65 (FIG. 1F), Ab101 (FIG. 1G), and Ab101-1 (FIG. 1H), two anti-CD19 VHHs and an anti-BCMA VHH were fused to each of the chains, in different configurations.

### Example 2. Comparison of B Cell Killing Efficiencies

This example tested certain multispecific antibodies' B cell killing capabilities.

In this assay, on day 0, human PBMCs were thawed and dispensed (2×10⁵ cells/well) into 96-well plates. The cells were incubated at 37 °C for 2 hours, and then treated with test antibodies or media for 48 hours. On day 2, B cells were counted and the killing ratios were calculated. Meanwhile, T cell activation was determined by FACS and cytokines release was measured.

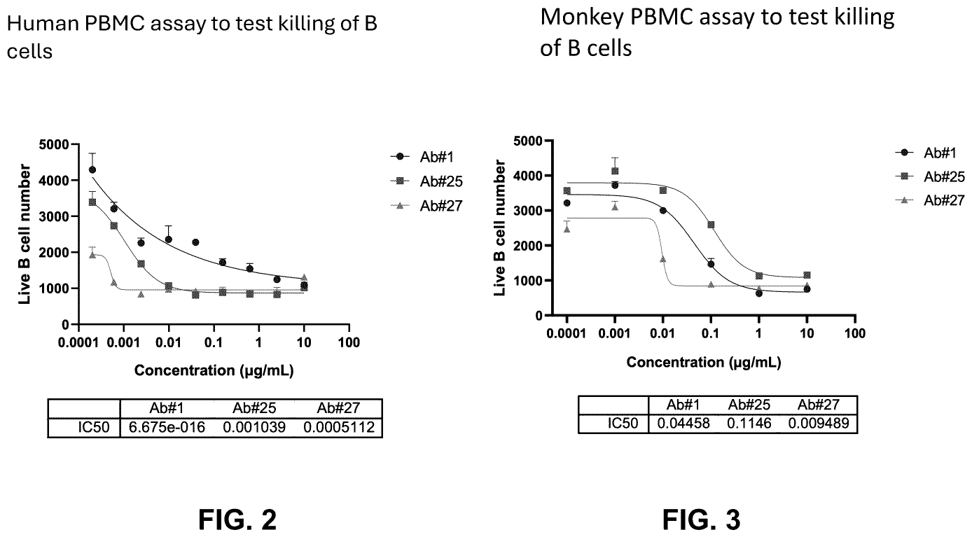

B cell killing was compared among Ab1, Ab25 and Ab27. As shown in FIG. 2, Ab27 (bivalent to CD19 and monovalent to BCMA) exhibited significantly higher human B cell killing than Ab25 (monovalent to both CD19 and BCMA), which in turn was more effective than Ab1 (monovalent to CD19).

Similar results were observed when these antibodies were tested against monkey PBMCs (FIG. 3). Ab27 exhibited significantly higher monkey B cell killing than Ab25, which in turn was more effective than Ab1.

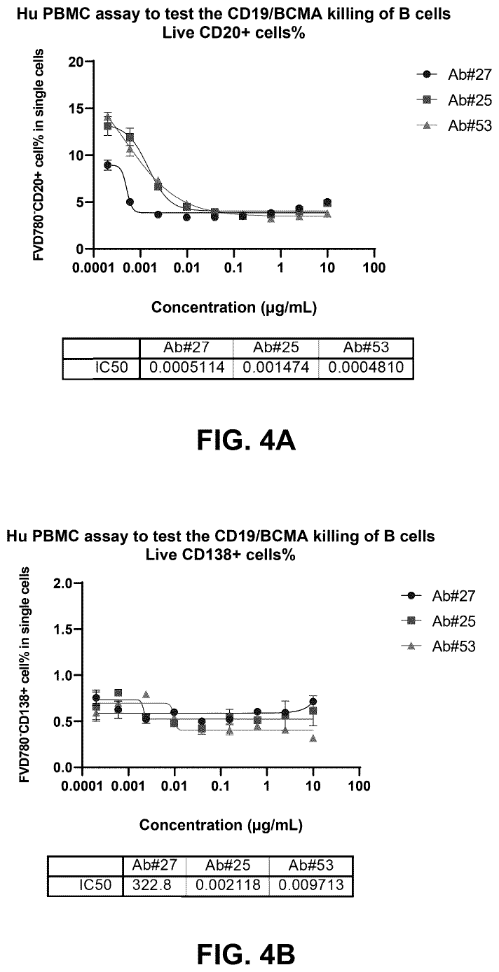

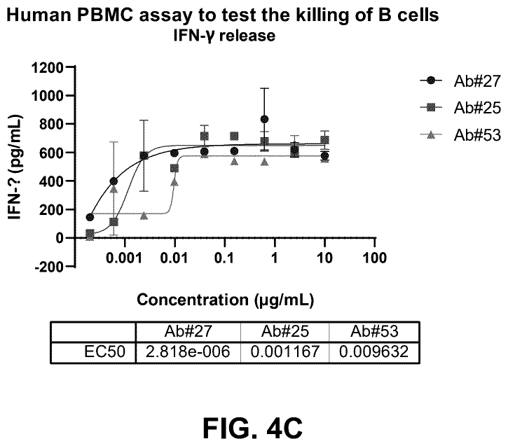

Another comparison was made, among Ab53 and Ab25 (monovalent to each of CD19 and BCMA), and Ab27 bivalent to CD19 and monovalent to BCMA). Ab27 was more potent than Ab53 and Ab25 in killing human B cells (FIGS. 4A and 4B). FIG. 4C shows IFN-7 release in PBMCs treated with Ab53, Ab25, and Ab27.

### Example 3. Tri-VHH Format had Less Cytokine Release than Bi-VHH Format

This example compared Ab22, Ab25, and Ab27 for their CD19+ leukemia cell killing and cytokine release activities.

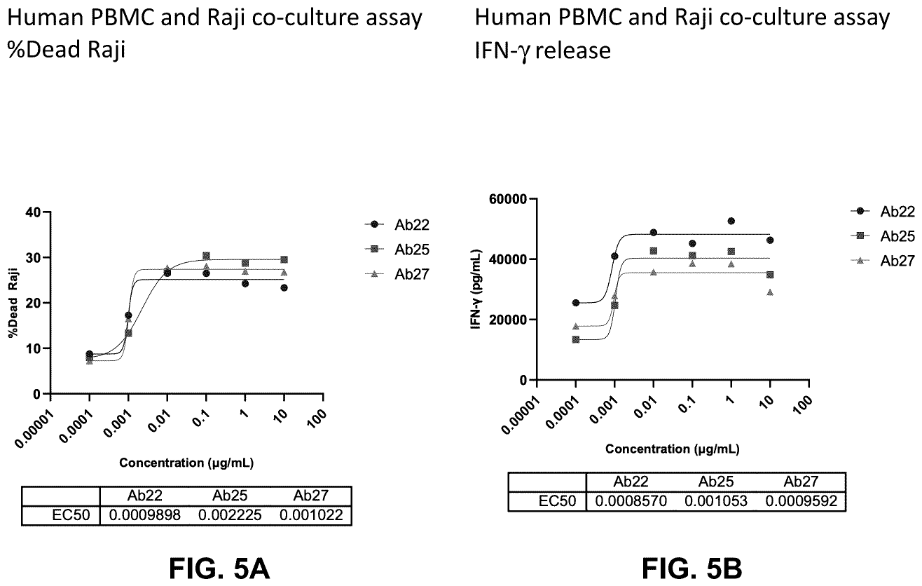

Ab22 included two anti-CD19 VHH on a single chain. Ab25 included two VHHs on the N-terminal ends of the anti-CD3 VH and VL. Ab27 included three VHHs. They were tested with CD19 positive leukemia cell Raji cells, and as shown in FIGS. 5A-B, Ab27 exhibited better cancer cell killing (FIG. 5A) with less cytokine release (FIG. 5B).

One of the main differences between Ab27 and Ab25 was that Ab27 is bivalent to CD19. This result, therefore, suggested that bivalent CD19 binding helped increase B cell killing with reduced cytokine release.

### Example 4. Ab101 was More Potent than Ab65

In this example, antibodies Ab1, Ab53, Ab65, and Ab101 were tested for their ability to kill CD20+ B cells, with Blincyto and teclistamab as references. Blincyto (blinatumomab) is a bispecific CD19-binding T cell engager. Teclistamab is a bispecific, BCMA-binding T cell engager.

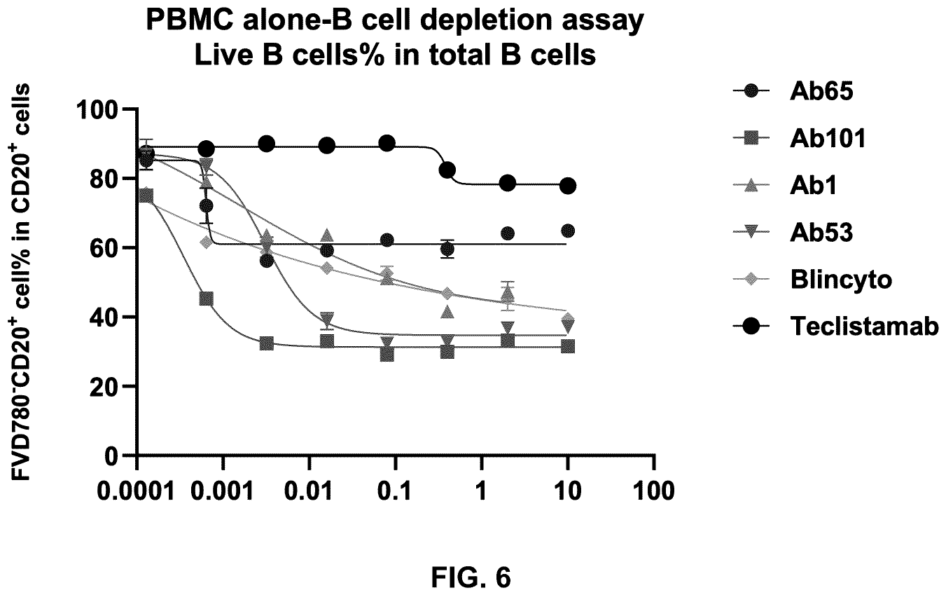

As shown in FIG. 6, Ab101 was considerably more active than all other tested antibodies, including Ab65. This was interesting, as Ab101 and Ab65 had very similar configurations, the main difference, beyond their distinct amino acid sequences, being that, in Ab101, the anti-CD3 VH was fused to an anti-CD19 VHH and the anti-CD3 VL was fused to an anti-BCMA VHH, whereas it was the reverse in Ab65. These results, therefore, suggest that when an anti-CD19 VHH was more distant from the anti-CD3 VH, their combinatory effect was more pronounced. All tested formats led to greater cytotoxicity than teclistamab, indicating that trispecific targeting of BCMA, CD19 and CD3 is more efficacious than bispecfic targeting of BCMA and CD3.

### Example 5. Further Evaluation of Ab101

The binding affinity of Ab101 to human CD19, BCMA, CD3ε proteins and cynomolgus CD3ε protein was tested here with Biacore®.

As shown in Table 3, Ab101 had strong binding to all of the tested proteins.

TABLE 3
Ab101 Binding

| | Human | Cyno |
|---|---|---|
| | KD(M) | KD(M) |
| CD3 | 5.52E−008 | ~5.0E−008 |
| CD19 | 1.75E−010 | binding |
| BCMA | 7.75E−08 | binding |

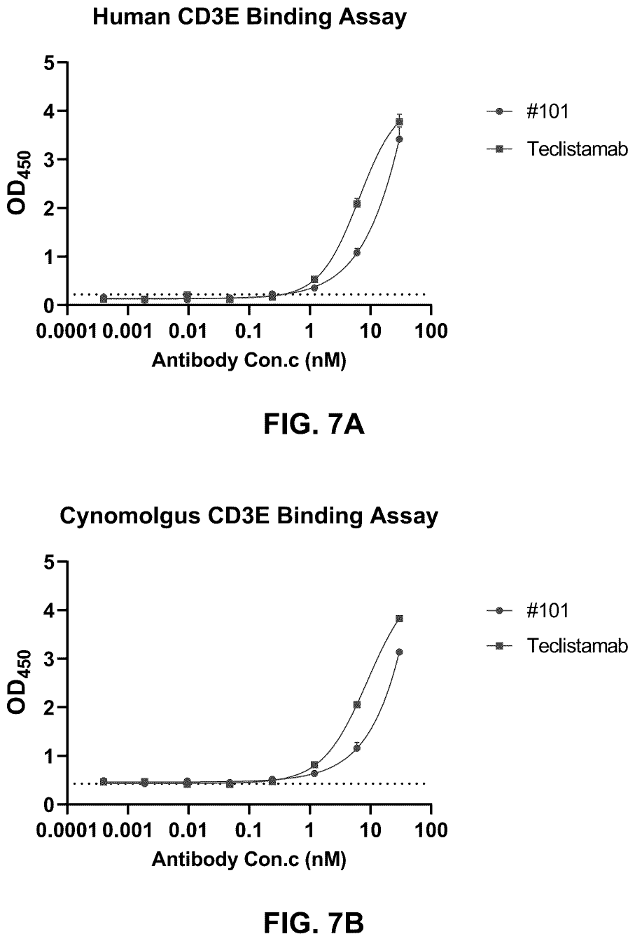

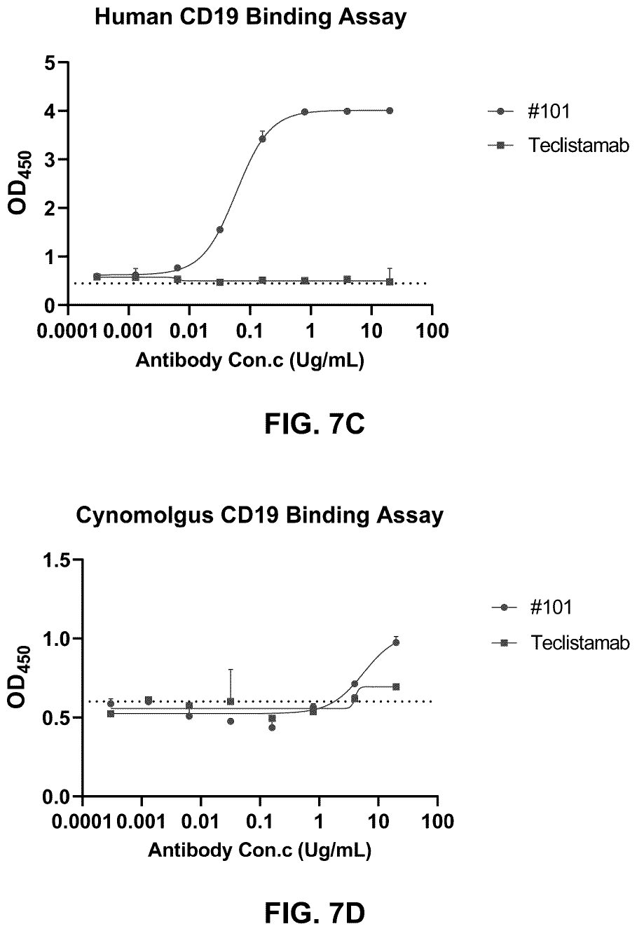

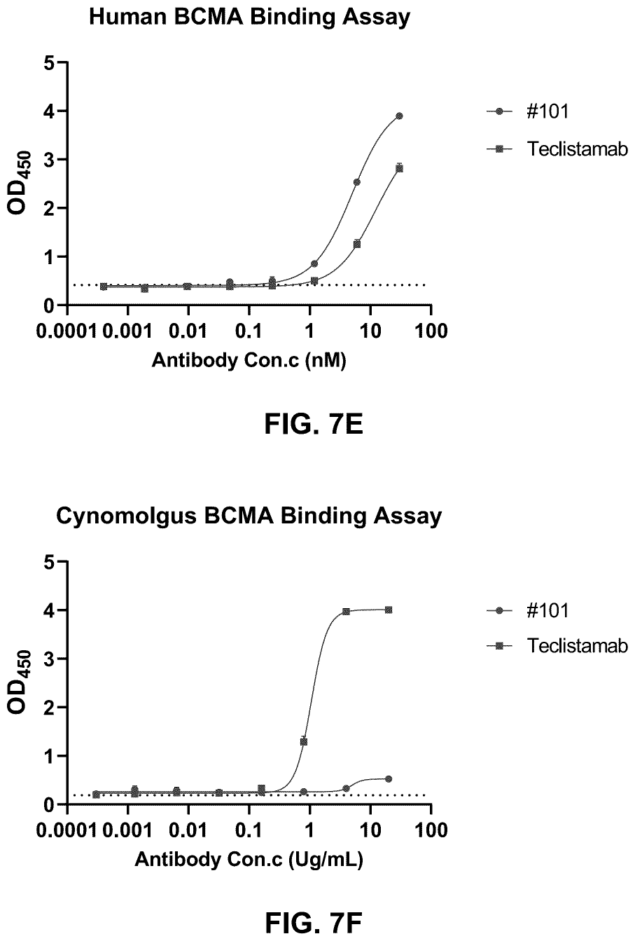

Affinity testing was also conducted with ELISA, and the results are shown in FIGS. 7A-F.

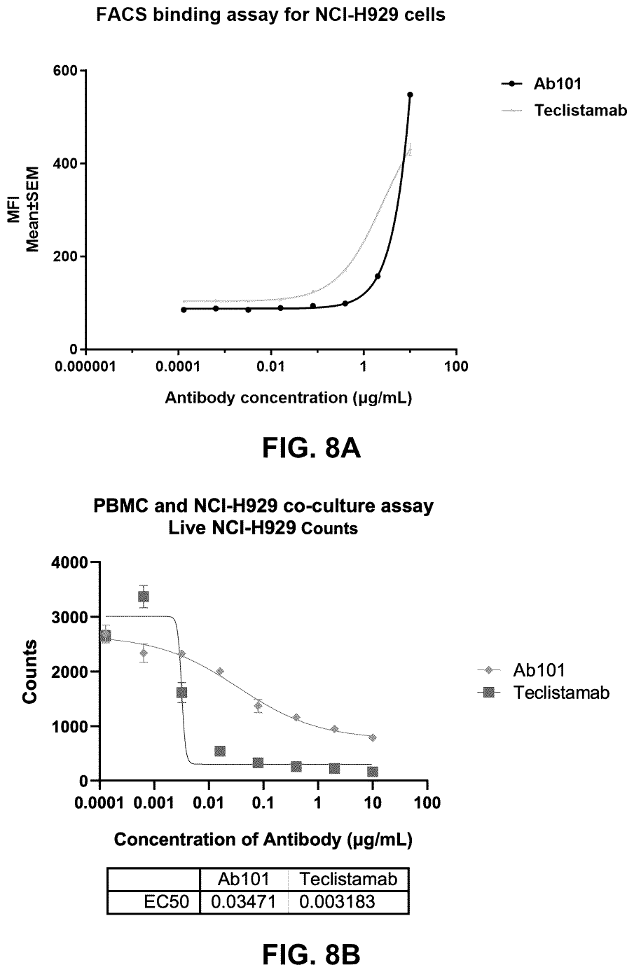

While Ab101 was only monovalent to BCMA, it exhibited potent killing of BCMA+ cells (NCI-929) (FIGS. 8A-B).

### Example 6. Excellent Therapeutic Index of Ab101 and Ab101-1

This example measured the antibodies' activity in depleting B cells in human and cyno PBMCs with blinatumomab as the benchmark.

On day 0, PBMCs were thawed and dispensed (2E5 cells/well) into 96-well plates. The samples were incubated at 37 °C for 2 hours, and then treated with the test antibodies (Ab #101, blinatumomab) or media for 48 hours. On day 2, B cell counts, killing ratio, and cytokines release were measured.

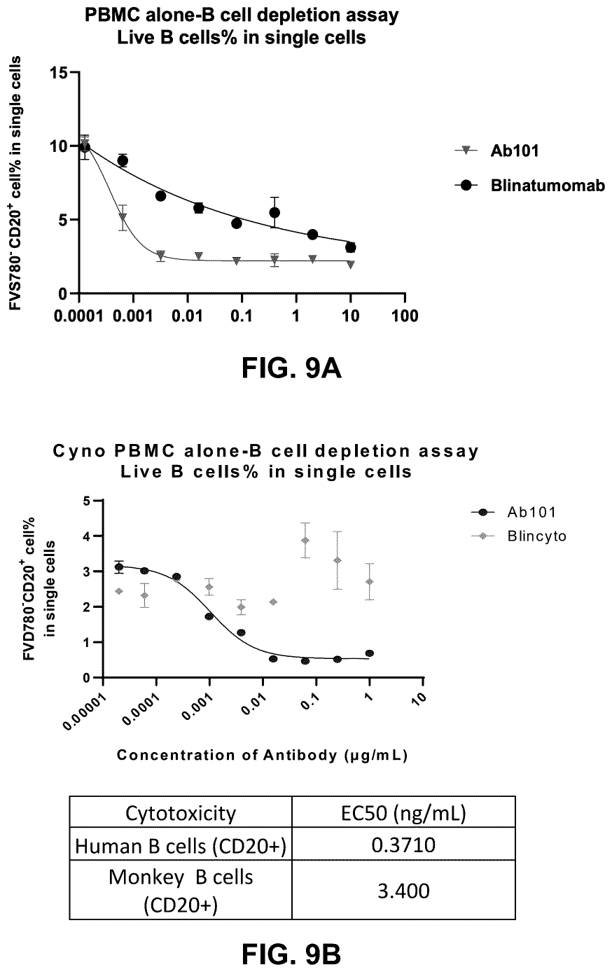

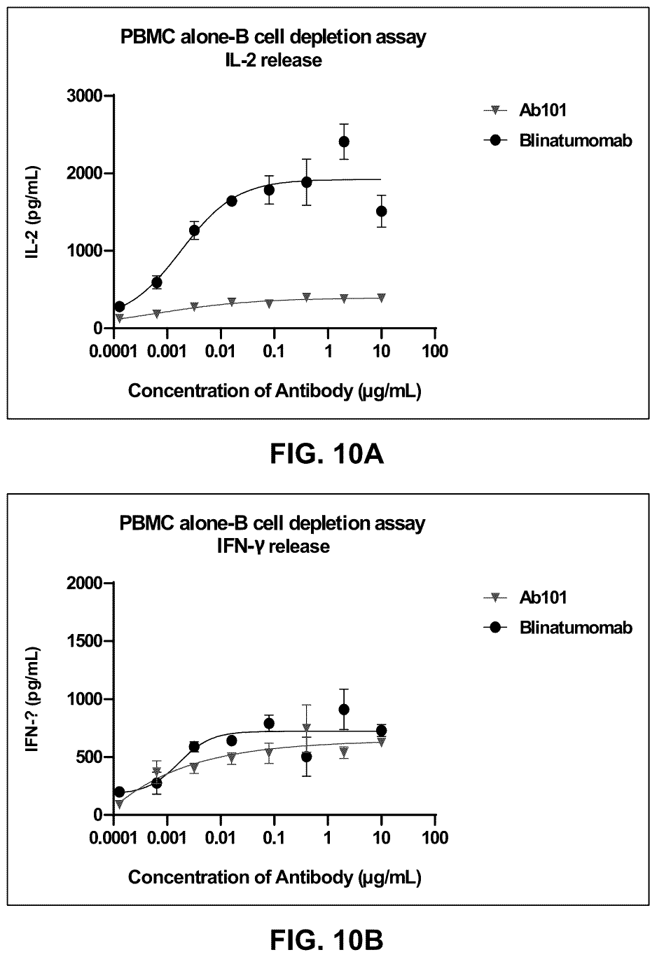

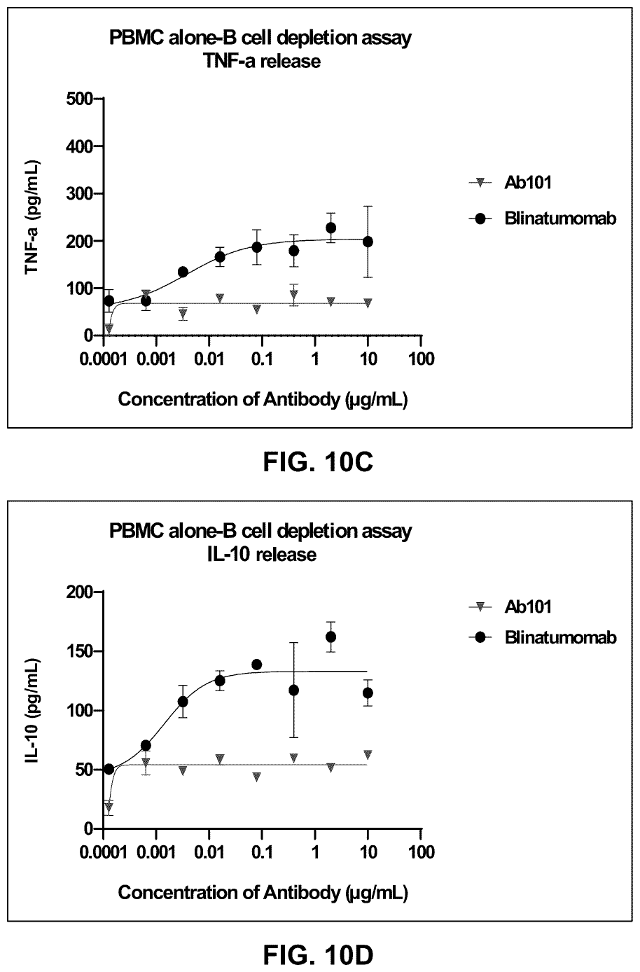

As shown in FIGS. 9A-B, Ab101 had significantly higher B cell depletion activity than blinatumomab. Quite impressively, however, Ab101 caused much less cytokine release than the benchmark (FIGS. 10A-D), demonstrating the excellent therapeutic index of the new antibody.

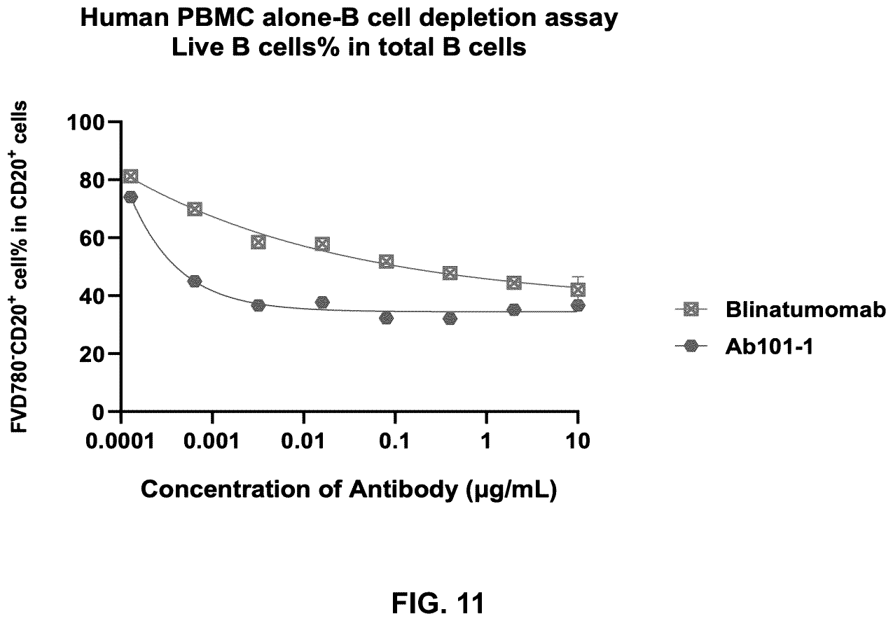

Similar potency was observed for Ab101-1 (FIG. 11), which included a slight variant of the anti-CD3 VL from Ab101.

### Example 7. Affinity of Ab101-1 to Human and Cyno CD3ε, Human CD19 and BCMA Proteins Assessed by BLI Method

In this Example, Bio-Layer Interferometry (BLI) was utilized to determine the binding affinity of Ab101-1 to human CD19 and BCMA, and human and cyno CD3ε.

The bio-layer interferometric molecular interaction instrument (GatorBio, Gator Prime) was used to detect and analyze the binding affinity of Ab101-1 with human CD3ε (ACRO, CD2-H5223), cynomolgus CD3ε (Kactus, CDE-CM101), human CD19 (Kactus CD1-HM119), and human BCMA (Kactus, BCM-117). The instrument was configured with the plate temperature set to 30 °C, the acquisition rate set to standard kinetics (50 Hz), and the probe speed set to 1000 rpm/min. The probe was pre-wetted by placing it in 250 μL/well of equilibration buffer [PBS (Solarbio, P1002), Tween-20 0.02% (v/v), (Solarbio, T8220), pH 7.2-7.4] added to the first column of the 96-well probe plate for 300 seconds. A probe baseline was then established by transferring the probe to the first column of the 96-well detection plate, which contained 200 μL/well of equilibration buffer, and allowing equilibration for 60 seconds.

For the loading step, the antibody or antigen was diluted to a working concentration of 5 μg/mL in equilibration buffer and added at 200 μL/well to the second column of the 96-well detection plate, with the probe set to load for 120 seconds. A loading baseline was subsequently recorded by equilibrating the probe for 60 seconds in the third column of the detection plate, which contained 200 μL/well of equilibration buffer.

Association kinetics were measured by preparing serial dilutions of the antigen or antibody in equilibration buffer across six concentrations. For antibody analytes, concentrations of 100 nM, 50 nM, 25 nM, 12.5 nM, 6.25 nM, and 0 nM were used, while for human CD19, concentrations of 200 nM, 100 nM, 50 nM, 25 nM, 12.5 nM, and 0 nM were prepared. These dilutions were added sequentially at 200 μL/well to the fourth column of the detection plate, and the probe was set to bind for 180 seconds. Dissociation was then measured by transferring the probe to the fifth column of the detection plate containing 200 μL/well of equilibration buffer and allowing dissociation to proceed for 300 seconds.

### Results:

TABLE 4
Affinity Study Results

| | | | | A | | | | | |
|---|---|---|---|---|---|---|---|---|---|
| | | | | Conc. | Koff | Kon | KD | | Full |
| Assay | Probe | Loading | Association | (nM) | (1/s) | (1/Ms) | (M) | Response | R² |
| 1 | Ni-NTA | Human | Ab101-1 | 100 | 3.24E−03 | 3.92E+04 | 8.27E−08 | 1.4801 | 0.998 |
| | | CD3& | | 50 | | | | 0.9965 | |
| | | His | | 25 | | | | 0.5762 | |
| | | | | 12.5 | | | | 0.2913 | |
| | | | | 6.25 | | | | 0.157 | |
| 2 | Ni-NTA | Cynomol | Ab101-1 | 100 | 1.97E−03 | 3.42E+04 | 5.77E−08 | 1.1151 | 0.999 |
| | | gus | | 50 | | | | 0.6589 | |
| | | CD3& | | 25 | | | | 0.3669 | |
| | | His | | 12.5 | | | | 0.1998 | |
| | | | | 6.25 | | | | 0.102 | |
| 3 | Protein A | Ab101-1 | Human | 200 | 1.06E−04 | 4.18E+04 | 2.54E−09 | 0.6759 | 0.998 |
| | | | CD19 | 100 | | | | 0.4876 | |
| | | | His | 50 | | | | 0.2929 | |
| | | | | 25 | | | | 0.16 | |
| | | | | 12.5 | | | | 0.0861 | |
| 4 | Ni-NTA | Human | Ab101-1 | 100 | 1.38E−03 | 2.11E+05 | 6.55E−09 | 4.5208 | 0.996 |
| | | BCMA | | 50 | | | | 3.7512 | |
| | | His | | 25 | | | | 2.7239 | |
| | | | | 12.5 | | | | 1.6205 | |
| | | | | 6.25 | | | | 0.9136 | |

High-affinity binding of Ab101-1 was observed to human CD19 (Kd: 2.54 nM, R²>0.99) and human BCMA (Kd: 6.55 nM, R²>0.99), with relatively lower binding to human CD3ε (Kd: 82.7 nM, R²>0.99). The binding profile of Ab101-1 to its targets, human CD19>human BCMA>human CD3ε, reflected an optimized design for selective B-cell depletion while potentially mitigating excessive T-cell activation. The Kd value of cynomolgus CD3 binding to Ab101-1 was 5.77E-08 M (R²>0.99).

### Example 8. Binding Assay of Ab101-1 to Human and Cynomolgus CD3e, CD19 and BCMA Proteins by ELISA

Enzyme-linked immunosorbent assay (ELISA) was used to detect and analyze the binding affinity of Ab101-1 to human CD3ε (Kactus, CDE-HM101), human CD19 (Kactus, CD1-HM119), human BCMA (Kactus, HM117), cynomolgus CD3ε (Kactus, CDE-CM101), cynomolgus CD19 (Kactus, CD1-CM119) and cynomolgus BCMA (Kactus, BCM-CM117). Human IgG1 Fc (Bio-Sincerity, YJ058-VH1) was used as the negative control.

### Study Method

Antigens were diluted in PBS to a working concentration of 10 μg/mL, and 100 μL was added per well to 96-well ELISA plates. Plates were incubated overnight at 4 °C in the dark. The following day, the antigen solution was discarded and the plates were washed three times with the washing buffer (PBS, 0.05% (v/v) Tween-20, pH 7.2-7.4). Blocking buffer (PBS supplemented with non-fat milk 5% (w/v), pH 7.2-7.4) was then added at 200 μL per well, and the plates were sealed and incubated at 37 °C for 1 hour in the dark. Antibodies were diluted in PBS to a working concentration series starting at 30 μg/mL, with 4-fold serial dilutions across 7 points. A volume of 100 μL per well was added to the ELISA plates, which were then incubated at 37 °C for 1 hour in the dark. Following incubation, the antibody solution was discarded and the plates were washed three times. A goat anti-human IgG Fc cross-adsorbed secondary antibody conjugated to HRP, diluted 1:2000 in PBS, was added at 100 μL per well. Plates were sealed and incubated at 37 °C for 1 hour in the dark. The secondary antibody solution was then discarded and the plates were washed three times with the microplate washer. Subsequently, 100 μL of Ultrasensitive TMB solution was added to each well and incubated at room temperature for 5 minutes. The colorimetric reaction was terminated by the addition of 100 μL of stop solution (1 M sulfuric acid (H₂SO₄) solution) per well, followed by thorough mixing. Absorbance was measured at OD450 using a microplate reader.

Data were processed using GraphPad Prism (version 8.3.0). A four-parameter logistic regression model ([Agonist] vs. Response—Variable Slope) was applied to analyze and plot the data, from which the half-maximal effective concentration (EC50) and maximum effective concentration (Emax) were derived for each condition.

### Results

TABLE 5
The OD₄₅₀ Value of antibodies bind to human CD3ε

| Assay-1 | Human CD3ε (10 μg/mL) | | | | | | | | |
|---|---|---|---|---|---|---|---|---|---|
| Ab (μg/mL) | 30 | 7.5 | 1.875 | 0.4688 | 0.1172 | 0.0293 | 0.0073 | 0.0000 | EC₅₀ |
| Ab101-1 | 2.2678 | 1.7596 | 0.9206 | 0.2991 | 0.1139 | 0.0648 | 0.0566 | 0.0499 | 3.031 |
| | 2.1491 | 1.7667 | 0.8630 | 0.2721 | 0.1087 | 0.0644 | 0.0518 | 0.0495 | |
| hIgG1 Fc | 0.0948 | 0.0630 | 0.0586 | 0.0545 | 0.0547 | 0.0520 | 0.0503 | 0.0559 | N/A |

TABLE 6
The OD₄₅₀ Value of antibodies bind to human CD19

| Assay-2 | Human CD19 (10 μg/mL) | | | | | | | | |
|---|---|---|---|---|---|---|---|---|---|
| Ab (μg/mL) | 30 | 7.5 | 1.875 | 0.4688 | 0.1172 | 0.0293 | 0.0073 | 0.0000 | EC₅₀ |
| Ab101-1 | 3.1616 | 3.0183 | 2.9758 | 2.8095 | 1.5090 | 0.5619 | 0.2251 | 0.0995 | 0.1229 |
| | 3.0985 | 2.9737 | 2.9066 | 2.5265 | 1.5302 | 0.5410 | 0.2229 | 0.0932 | |
| hIgG1 Fc | 0.1025 | 0.0888 | 0.0800 | 0.0725 | 0.0699 | 0.0703 | 0.0742 | 0.0825 | N/A |

TABLE 7
The OD₄₅₀ Value of antibodies bind to human BCMA

| Assay-3 | Human BCMA (10 μg/mL) | | | | | | | | |
|---|---|---|---|---|---|---|---|---|---|
| Ab (μg/mL) | 30 | 7.5 | 1.875 | 0.4688 | 0.1172 | 0.0293 | 0.0073 | 0.0000 | EC₅₀ |
| Ab101-1 | 3.7917 | 3.6432 | 3.1126 | 1.8932 | 0.5094 | 0.1507 | 0.2078 | 0.0855 | 0.546 |
| | 3.7952 | 3.7197 | 3.0299 | 1.8008 | 0.5131 | 0.2360 | 0.1111 | 0.0907 | |
| hIgG1 Fc | 0.1595 | 0.0810 | 0.0639 | 0.1086 | 0.0577 | 0.0985 | 0.0612 | 0.0675 | N/A |

TABLE 8
The OD₄₅₀ Value of antibodies bind to cynomolgus CD38

| Assay-4 | Cynomolgus CD3ε (10 μg/mL) | | | | | | | | |
|---|---|---|---|---|---|---|---|---|---|
| Ab (μg/mL) | 30 | 7.5 | 1.875 | 0.4688 | 0.1172 | 0.0293 | 0.0073 | 0.0000 | EC₅₀ |
| Ab101-1 | 3.6440 | 3.1601 | 1.7225 | 0.5008 | 0.1453 | 0.0897 | 0.1226 | 0.1287 | 2.275 |
| | 3.5738 | 3.0472 | 1.6424 | 0.4738 | 0.1967 | 0.0993 | 0.1108 | 0.1111 | |
| hIgG1 Fc | 0.1253 | 0.0917 | 0.0812 | 0.1055 | 0.0818 | 0.0763 | 0.1115 | 0.0903 | N/A |

TABLE 9
The OD₄₅₀ Value of antibodies bind to Cynomolgus CD19

| Assay-5 | Cynomolgus CD19 (10 μg/mL) | | | | | | | | |
|---|---|---|---|---|---|---|---|---|---|
| Ab (μg/mL) | 30 | 7.5 | 1.875 | 0.4688 | 0.1172 | 0.0293 | 0.0073 | 0.0000 | EC₅₀ |
| Ab101-1 | 3.6973 | 1.7485 | 0.2646 | 0.1155 | 0.0884 | 0.065 | 0.0667 | 0.4336 | 10.44 |
| | 2.0018 | 0.7735 | 0.229 | 0.0998 | 0.0856 | 0.0738 | 0.069 | 0.077 | |
| hIgG1 Fc | 0.2237 | 0.1591 | 0.1142 | 0.1157 | 0.0976 | 0.0973 | 0.0915 | 0.0918 | N/A |

TABLE 10
The OD₄₅₀ Value of antibodies bind to Cynomolgus BCMA

| Assay-6 | Cynomolgus BCMA (10 μg/mL) | | | | | | | | |
|---|---|---|---|---|---|---|---|---|---|
| Ab (μg/mL) | 30 | 7.5 | 1.875 | 0.4688 | 0.1172 | 0.0293 | 0.0073 | 0.0000 | EC₅₀ |
| Ab101-1 | 0.5088 | 0.403 | 0.3516 | 0.2568 | 0.1074 | 0.0738 | 0.0725 | 0.0726 | 2.369 |
| | 0.4944 | 0.4717 | 0.1792 | 0.2514 | 0.0789 | 0.0765 | 0.0868 | 0.0696 | |
| hIgG1 Fc | 0.1135 | 0.1490 | 0.0954 | 0.1828 | 0.1211 | 0.0573 | 0.1456 | 0.1166 | N/A |

Ab101-1 was able to bind to human CD3ε, the EC50 value was 3.031 μg/mL. Ab101-1 was able to bind to human CD19, the EC50 value was 0.1229 μg/mL. Ab101-1 was able to bind to human BCMA, the EC50 value was 0.546 μg/mL. Ab101-1 was able to bind to cynomolgus CD3ε, the EC50 value was 2.275 μg/mL. Ab101-1 was able to bind to cynomolgus CD19, the EC50 value was 10.44 μg/mL. Ab101-1 was able to bind to cynomolgus BCMA, the EC50 value was 2.369 μg/mL.

The results showed i) that Ab-101-1 demonstrated comparable binding to both human and cynomolgus CD3R; and ii) Ab101-1 bound to human CD19 and BCMA more effectively than to cynomolgus CD19 and BCMA, respectively.

### Example 9. Binding of Ab101-1 to Human FcγRIIIa, FcγRIIIb, FcγRIIa, FcγRIIb/c, and FcγRI Proteins by ELISA

The binding affinity of Ab101-1 to key human FcγRs was evaluated using enzyme linked immunosorbent assay (ELISA). The panel included low-affinity receptors CD16a (FcγRIIIa) (Kactus, FRI-HM43AB), CD16b (FcγRIIIb) (Kactus, FCR-HM42BB), CD32a (FcγRIIa) (Kactus, CDA-HM432B), CD32b/c (FcγRIIb/c) (Kactus, CDB-HM464B), as well as high-affinity receptor CD64 (FcγRI) (Kactus, FRI-HM464B). Rituximab, a known FcγR-binding antibody, was included as a positive control.

The antibody Ab101-1 was diluted in PBS to a starting working concentration of 4 μg/mL, followed by 2-fold serial dilutions to generate 7 concentration gradients. A volume of 100 μL per well was added to a 96-well ELISA plate, which was then sealed with sealing film and incubated overnight at 4 °C in the dark. The following day, the antibody solution was discarded and the plate was washed (PBS, Tween 20 0.05% v/v, pH 7.2-7.4) three times using a microplate washer. Blocking buffer (PBS, 5% non-fat milk, pH 7.2-7.4) was added at 200 μL per well, and the plate was sealed and incubated at 37 °C for 1 hour in the dark. Antigens were diluted in PBS to 2 μg/mL (yielding a final concentration of 1 μg/mL), and streptavidin-HRP was diluted in PBS to 1:10,000 (yielding a final concentration of 1:20,000). These two components were mixed in equal volumes, and the mixture was sealed and incubated at 37 °C for 1 hour in the dark. The antibody solution was then discarded from the ELISA plate, and the plate was washed three times. The antigen and streptavidin-HRP mixture was added at 100 μL per well, and the plate was sealed and incubated at 37 °C for 1 hour in the dark. Following incubation, the antigen and streptavidin-HRP mixture was discarded and the plate was washed three times. Subsequently, 100 μL of Ultrasensitive TMB solution (Solarbio, PR1200) was added to each well and incubated at room temperature for 5 minutes. The colorimetric reaction was terminated by adding 100 μL of stop solution to each well with thorough shaking. Absorbance was measured at OD450 using a microplate reader.

Data were processed using GraphPad Prism (version 8.3.0). A four-parameter variable slope regression model ([agonist] vs. response) was applied for curve fitting, from which the half-maximal effective concentration (EC50) and maximum effect (Emax) values were calculated.

### Results

TABLE 11
The OD₄₅₀ Value of antibodies bind to human CD16a(FcγRIIIa)

| Assay-1 | Human CD16a (FcγRIIIa) | | | | | | | | | |
|---|---|---|---|---|---|---|---|---|---|---|
| Ab(μg/mL) | 4 | 2 | 1 | 0.5 | 0.25 | 0.125 | 0.0625 | 0 | EC₅₀ | Eₘₐₓ |
| Rituximab | 2.9209 | 0.8951 | 0.1107 | 0.0709 | 0.0728 | 0.0722 | 0.1122 | 0.1347 | 2.385 | 4.108 |
| | 2.6151 | 0.8826 | 0.1327 | 0.081 | 0.0771 | 0.0809 | 0.0928 | 0.116 | | |
| | 2.6326 | 0.8447 | 0.1113 | 0.0783 | 0.0834 | 0.0868 | 0.092 | 0.117 | | |
| Ab101-1 | 0.0692 | 0.0846 | 0.0771 | 0.0769 | 0.0811 | 0.0783 | 0.0902 | 0.1164 | N/A | N/A |
| | 0.0696 | 0.0658 | 0.1246 | 0.0784 | 0.0754 | 0.0775 | 0.0806 | 0.1027 | | |
| | 0.0667 | 0.109 | 0.1184 | 0.0796 | 0.0859 | 0.0782 | 0.0891 | 0.1054 | | |

TABLE 12
The OD₄₅₀ Value of antibodies bind to human CD16b(FcγRIIIb)

| Assay-2 | Human CD16b (FcγRIIIb) | | | | | | | | | |
|---|---|---|---|---|---|---|---|---|---|---|
| Ab(μg/mL) | 4 | 2 | 1 | 0.5 | 0.25 | 0.125 | 0.0625 | 0 | EC₅₀ | Eₘₐₓ |
| Rituximab | 1.1788 | 0.5419 | 0.3991 | 0.3182 | 0.3683 | 0.2500 | 0.2807 | 0.2510 | 47.180 | N/A |
| | 0.8676 | 0.7107 | 0.4037 | 0.3004 | 0.3526 | 0.2383 | 0.2244 | 0.1948 | | |
| | 0.8340 | 0.4314 | 0.2301 | 0.1337 | 0.1208 | 0.0992 | 0.1309 | 0.0953 | | |
| Ab101-1 | 0.2773 | 0.3347 | 0.1551 | 0.0924 | 0.1077 | 0.0980 | 0.1080 | 0.0810 | 1.041 | N/A |
| | 0.0890 | 0.1522 | 0.0793 | 0.0801 | 0.0738 | 0.0713 | 0.0883 | 0.0833 | | |
| | 0.0689 | 0.1409 | 0.0806 | 0.0771 | 0.0888 | 0.0744 | 0.0589 | 0.0660 | | |

TABLE 13
The OD₄₅₀ Value of antibodies bind to human CD32a(FcγRIIa)

| Assay-3 | Human CD32a (FcγRIIa) | | | | | | | | | |
|---|---|---|---|---|---|---|---|---|---|---|
| Ab(μg/mL) | 4 | 2 | 1 | 0.5 | 0.25 | 0.125 | 0.0625 | 0 | EC₅₀ | Eₘₐₓ |
| Rituximab | 3.9414 | 2.0144 | 0.3692 | 0.1008 | 0.0729 | 0.0613 | 0.0710 | 0.0837 | 2.129 | 4.822 |
| | 3.9524 | 2.1040 | 0.4358 | 0.9386 | 0.0777 | 0.0711 | 0.0686 | 0.0652 | | |
| | 3.911 | 1.783 | 0.2104 | 0.0832 | 0.0725 | 0.0727 | 0.0732 | 0.0690 | | |
| Ab101-1 | 0.0666 | 0.0697 | 0.0685 | 0.0694 | 0.0711 | 0.0704 | 0.0724 | 0.0845 | N/A | N/A |
| | 0.0655 | 0.1510 | 0.0996 | 0.0776 | 0.0708 | 0.0684 | 0.0717 | 0.0699 | | |
| | 0.0584 | 0.0583 | 0.0575 | 0.0828 | 0.0673 | 0.0615 | 0.0649 | 0.0626 | | |

TABLE 14
The OD₄₅₀ Value of antibodies bind to human CD32b/c(FcγRIIb/c)

| Assay-4 | Human CD32b/c (FcγRIIb/c) | | | | | | | | | |
|---|---|---|---|---|---|---|---|---|---|---|
| Ab(μg/mL) | 4 | 2 | 1 | 0.5 | 0.25 | 0.125 | 0.0625 | 0 | EC₅₀ | Eₘₐₓ |
| Rituximab | 3.6309 | 1.2476 | 0.1851 | 0.0849 | 0.0696 | 0.0746 | 0.1195 | 0.0938 | 2.409 | 4.802 |
| | 3.3807 | 1.1867 | 0.1657 | 0.0728 | 0.0739 | 0.0926 | 0.0830 | 0.0649 | | |
| | 3.2863 | 1.3197 | 0.1577 | 0.0941 | 0.0664 | 0.0755 | 0.0871 | 0.0737 | | |
| Ab101-1 | 0.3912 | 0.1511 | 0.082 | 0.0813 | 0.0650 | 0.0673 | 0.0766 | 0.1103 | 2.756 | N/A |
| | 0.3885 | 0.1585 | 0.0922 | 0.0853 | 0.0728 | 0.0781 | 0.0742 | 0.0760 | | |
| | 0.3759 | 0.1407 | 0.0805 | 0.0890 | 0.0838 | 0.0828 | 0.0887 | 0.0927 | | |

TABLE 15
The OD₄₅₀ Value of antibodies bind to human CD64(FcγRI)

| Assay-5 | Human CD64(FcγRI) | | | | | | | | | |
|---|---|---|---|---|---|---|---|---|---|---|
| Ab(μg/mL) | 4 | 2 | 1 | 0.5 | 0.25 | 0.125 | 0.0625 | 0 | EC₅₀ | Eₘₐₓ |
| Rituximab | 4.0000 | 3.7852 | 2.3831 | 0.9087 | 0.5159 | 0.2452 | 0.1837 | 0.1082 | 0.976 | 3.249 |
| | 4.0000 | 3.7857 | 1.9586 | 0.8745 | 0.4193 | 0.2234 | 0.1532 | 0.0954 | | |
| | 4.0000 | 3.7704 | 2.1114 | 0.9275 | 0.446 | 0.2125 | 0.1300 | 0.0681 | | |
| Ab101-1 | 0.0838 | 0.0680 | 0.0619 | 0.0584 | 0.0602 | 0.0604 | 0.0606 | 0.0591 | N/A | N/A |
| | 0.0809 | 0.0621 | 0.0605 | 0.059 | 0.0620 | 0.0597 | 0.0611 | 0.0662 | | |
| | 0.0838 | 0.0696 | 0.0650 | 0.0591 | 0.0604 | 0.0645 | 0.0613 | 0.0614 | | |

Rituximab bound to human CD16a (FcγRIIIa), CD16b (FcγRIIIb), CD32a (FcγRIIa), CD32b/c (FcγRIIb/c), and CD64(FcγRI), with EC50 values of 2.385 μg/mL, 47.18 μg/mL, 2.129 μg/mL, 2.409 μg/mL, and 0.976 μg/mL, respectively. By contrast, Ab101-1 showed no detectable binding to any of the tested human FcγRs, and the EC₅₀ values could not be determined.

Ab101-1 showed no detectable binding to human Fcγ receptors, confirming that Fc engineering successfully eliminated FcγR interactions and thereby minimized the potential for Fc-mediated effector functions such as ADCC and ADCP.

### Example 10. Affinity of Ab101-1 to Human FcRn Assessed by BLI Method

In this study, Bio-Layer Interferometry (BLI) was used to assess the binding affinity of antibody Ab101-1 to human FcRn, with trastuzumab included as a control.

### Study Methods

The instrument (Gator Prime) was prepared by setting the plate temperature to 30 °C, the acquisition rate to standard kinetics (50 Hz), and the probe speed to 1000 rpm/min. Equilibration buffer was added at 250 μL per well to the first column of the 96-well probe plate. The probe was placed in the equilibration buffer (PBS, Tween-20 0.02% v/v, pH 7.2-7.4) and pre-wetted for 300 seconds. To establish the probe baseline, 200 μL per well of equilibration buffer was added to the first column of the 96-well detection plate, and the probe was equilibrated for 60 seconds. For loading, the antigen (human FcRn) was diluted to a working concentration of 5 μg/mL in equilibration buffer and added at 200 μL per well to the second column of the 96-well detection plate. The antigen was loaded for 120 seconds. Following loading, a baseline was established by adding 200 μL per well of equilibration buffer to the third column of the 96-well detection plate and equilibrating the probe for 60 seconds. For the association phase, the antibodies (trastuzumab and Ab101-1) were diluted in equilibration buffer to generate six concentrations (400 nM, 200 nM, 100 nM, 50 nM, 25 nM, and 0 nM). These were added sequentially at 200 μL per well to the fourth column of the 96-well detection plate, and binding was allowed to proceed for 180 seconds. Dissociation was initiated by adding 200 μL per well of equilibration buffer to the fifth column of the 96-well detection plate and allowing dissociation to occur for 300 seconds. Data analysis was performed according to the manufacturer instructions.

### Results

TABLE 16
Affinity Study Results

| | | | | A | | | | | |
|---|---|---|---|---|---|---|---|---|---|
| | | | | Conc. | Koff | Kon | KD | | Full |
| Assay | Probe | Loading | Association | (nM) | (1/s) | (1/Ms) | (M) | Response | R² |
| 1 | Ni-NTA | Human | Trastuzumab | 400 | 1.82E−03 | 4.29E+04 | 4.24E−08 | 0.1356 | 0.953 |
| | | FcRn | | 200 | | | | 0.0803 | |
| | | His | | 100 | | | | 0.0356 | |
| | | | | 50 | | | | 0.0187 | |
| | | | | 25 | | | | 0.0174 | |
| 2 | Ni-NTA | Human | Ab101-1 | 400 | 6.14E−04 | 2.20E+04 | 2.80E−08 | 0.297 | 0.993 |
| | | FcRn | | 200 | | | | 0.1634 | |
| | | His | | 100 | | | | 0.0838 | |
| | | | | 50 | | | | 0.0418 | |
| | | | | 25 | | | | 0.0345 | |

The K_{d} value of Human FcRn binding to trastuzumab was 4.24E-08 M; R₂=0.953. The K_{d} value of Human FcRn binding to Ab101-1 was 2.80E-08 M. R₂=0.993.

Ab101-1 retained the strong binding to human FcRn, with a dissociation constant (Kd) of 28 nM, consistent with a typical IgG antibody in vivo.

### Example 11. Flow Cytometry Binding Assay with Human and Cynomolgus PBMCs

The objective of this study was to evaluate the binding activities of Ab101-1 and teclistamab to human and cynomolgus (cyno) CD4+ T cells, CD8+ T cells, and CD20+ B cells.

### Methods:

Human and cyno peripheral blood mononuclear cells (PBMCs) were prepared and resuspended in RPMI-1640 complete medium. The cells were dispensed into the designated wells of 96-well plates and incubated with test articles for 1 hour at 4 °C. Subsequently, the cells were washed with Cell Staining Buffer (BioLegend, 420201) and stained with FITC-conjugated anti-human CD4 (BioLegend, 317408), Alexa Fluor 700-conjugated anti-human CD8 (BioLegend, 344724), BV421-conjugated anti-human CD20 (BioLegend, 302330), and PE-conjugated anti-human IgG working solutions (BioLegend, 398004) at 4 °C for 30 minutes. After two additional washes with Cell Staining Buffer, the mean fluorescence intensity (MFI) values of the PE channel for CD4+ T cells, CD8+ T cells, and CD20+ B cells were quantified by flow cytometry.

### Statistical Analysis

Raw data were analyzed and exported using Attune Cytometric Software (Thermo Fisher Scientific, version 6.2.2). Subsequent data analysis was performed using Microsoft Office Excel 2021 and GraphPad Prism 10. EC50 was calculated using equation log(agonist) vs. response—Variable slope (four parameters) in GraphPad Prism 10. Data are presented as mean±SEM.

### Results:

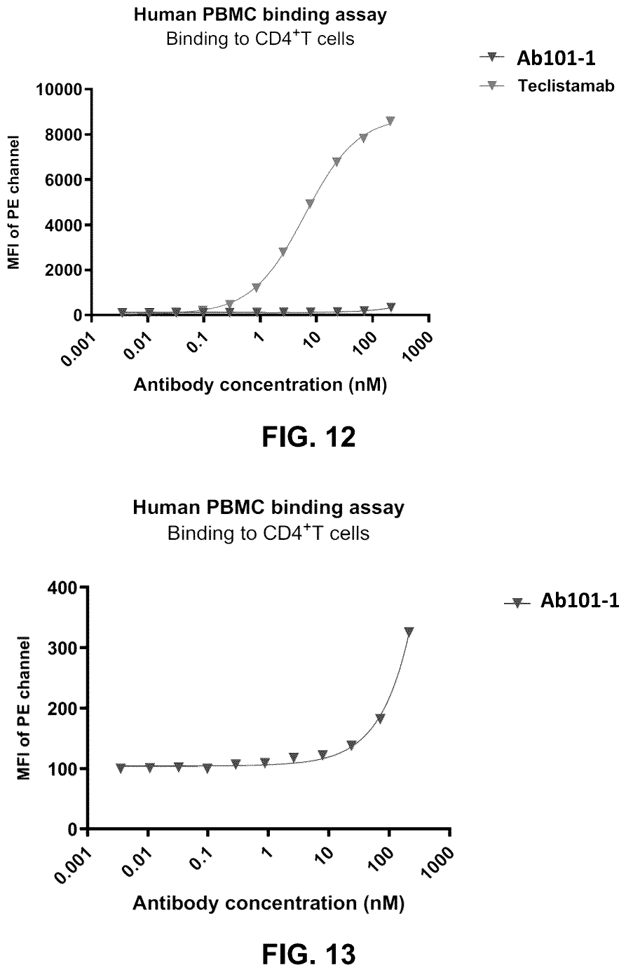

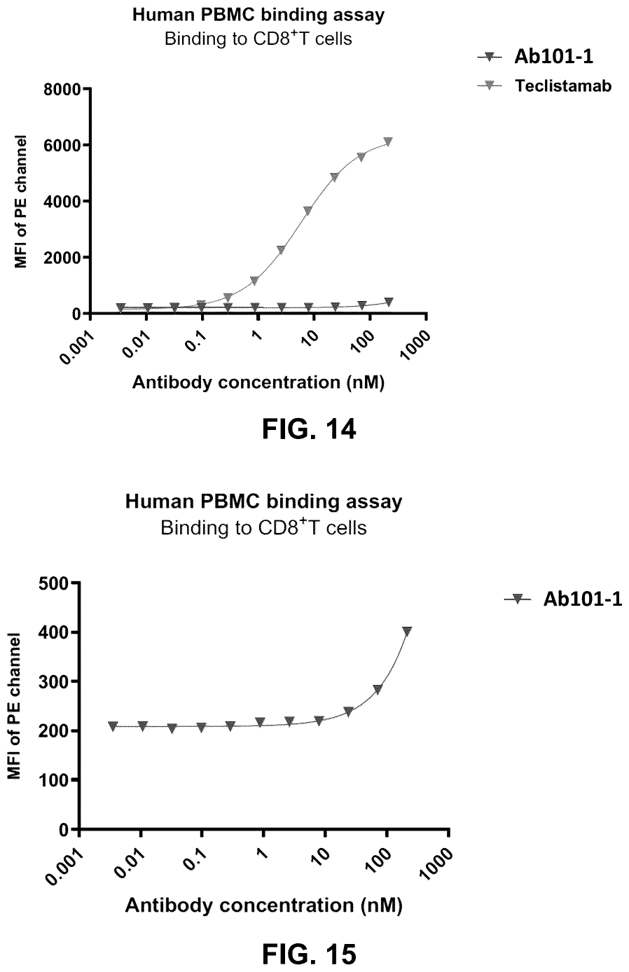

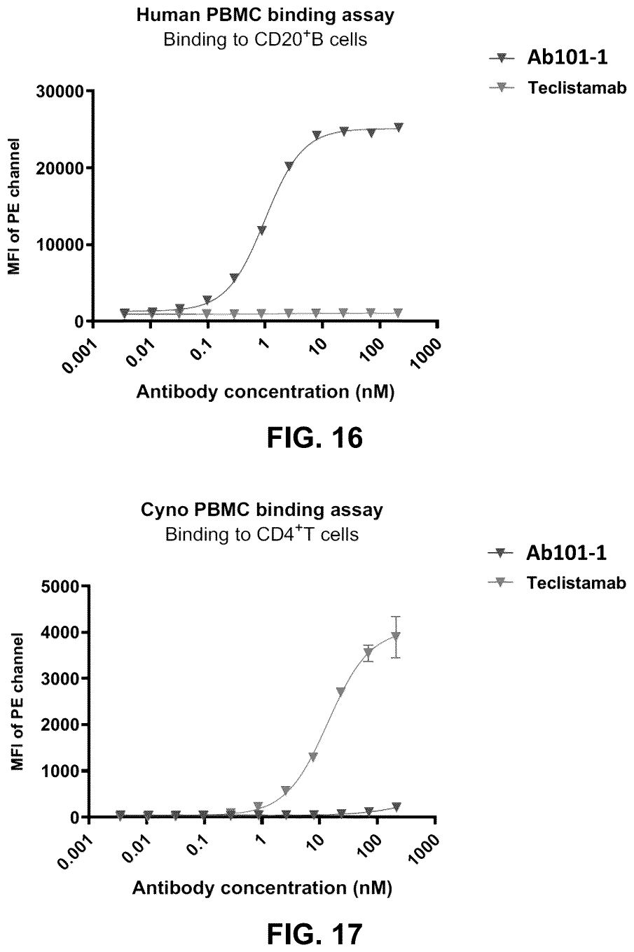

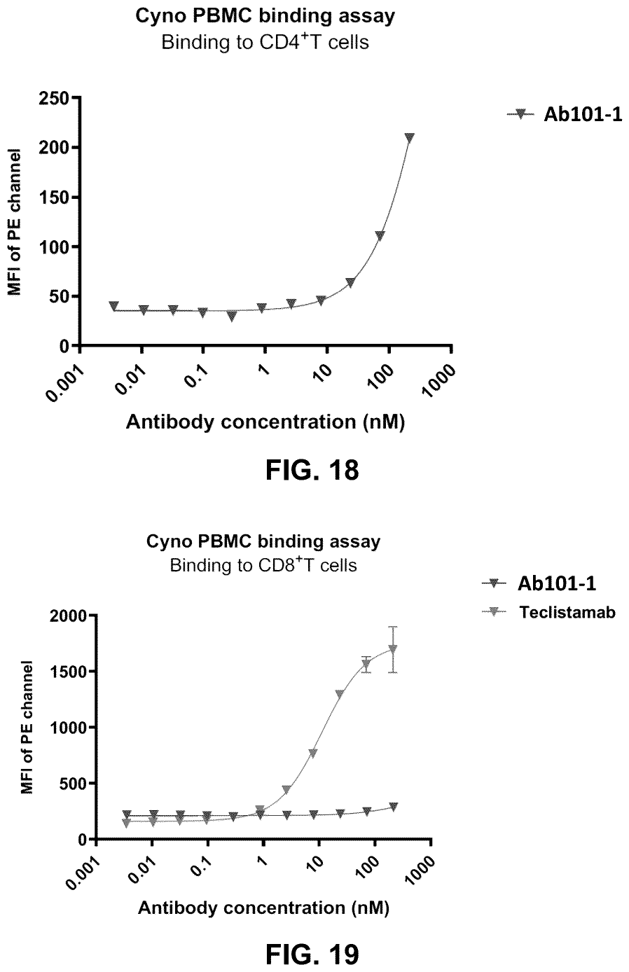

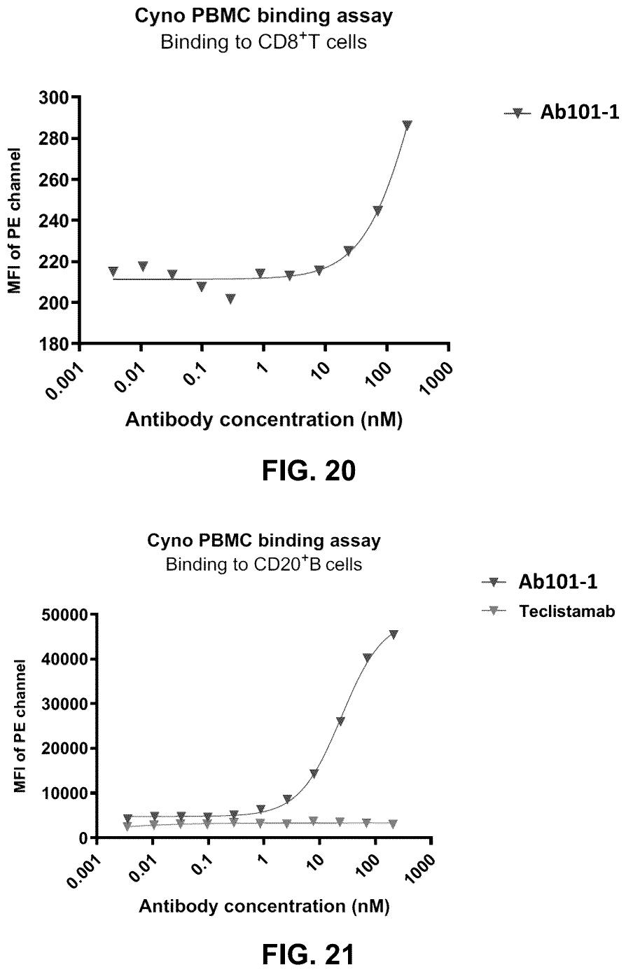

Ab101-1 exhibited dose-dependent binding to human and cyno CD4+ T cells (FIGS. 12-13 (human), FIGS. 17-18 (cyno)), CD8+ T cells (FIGS. 14-15 (human), FIGS. 19-20 (cyno), and CD20+ B cells (FIG. 16 (human), FIG. 21 (cyno)). Much stronger binding to human CD20+ B cells (EC50 0.99 nM) compared to cyno CD20+ B cells (EC50 24.52 nM) was observed. Teclistamab demonstrated dose-dependent binding only to human and cynomolgus CD4+ T cells and CD8+ T cells. Relatively weaker binding of Ab101-1 than teclistamab was observed for both human and cynomolgus CD4+ T cells and CD8+ T cells.

### Example 12. T Cell Activation Assay of Ab101-1

To evaluate Ab101-1's T cell activation potential, a reporter gene assay was carried out using target cells Raji cells (a CD19+ B-cell leukemia line), and engineered Jurkat NFAT-Luc cells (a T-cell line with an NFAT-driven luciferase reporter). Blinatumomab, a clinically validated CD19×CD3 bispecific antibody, was used as control.

In this Example, Ab101-1 demonstrated potent T cell activation with EC50 of 0.2518 nM, which was an order of magnitude less potent than blinatumomab (EC50=0.0135 nM), suggesting Ab101-1 may potentially have a lower risk of cytokine release syndrome in patients. Detailed dose-response study and analyses are provided in the Results section.

### Study Methods

Target cells were cultured in RPMI 1640 complete medium (RPMI 1640 supplemented with 10% fetal bovine serum (FBS) and 1% penicillin-streptomycin) until reaching the logarithmic growth phase, then resuspended in the same medium at a density of 5×10⁵ cells/mL. A total of 2×10⁴ target cells (40 μL) were dispensed into each well of a 96-well round-bottom plate, followed by the addition of 20 μL of 5× test antibody solutions. The antibodies were prepared at a starting final concentration of 5 nM, with 5-fold serial dilutions across 8 concentration points. Jurkat NFAT-Luc Reporter cells (COBIOER, CBP74025) were cultured separately in reporter cells medium (RPMI 1640 supplemented with 10% FBS and 800 μg/mL hygromycin) until reaching the logarithmic growth phase, then resuspended in RPMI 1640 complete medium at a density of 2.5×10⁶ cells/mL. A total of 1×10⁵ NFAT-Luc Jurkat Reporter cells (40 μL) were dispensed into each well of the 96-well round-bottom plate. The plate was then incubated at 37 °C for 16 hours.

The detection reagent was prepared by mixing the substrate solution and lysis substrate solution from the Ultra Luciferase Detection Kit (COBIOER, CBPH0001) at a volume ratio of 1:50. Following the 16-hour incubation, 100 μL per well of the cell lysis/substrate solution was added to each well of the 96-well plate and incubated at room temperature for 5 minutes. Luminescence values were subsequently recorded using a microplate reader with a measurement interval of 500 ms.

### Results

Measurements are shown in Table 17.

TABLE 17
Luminescence (RLU) of Jurkat NFAT Luc Reporter Cell (Raji as the target cells)

| | Effector cell: Jurkat NFAT Luc Reporter cell | | | | | | | | |
|---|---|---|---|---|---|---|---|---|---|
| Assay | Target cell: Raji | | | | | | | | |
| Ab (nM) | 5 | 1 | 0.2 | 0.04 | 0.008 | 0.0016 | 0.0003 | 0 | EC50 |
| Blinatumomab | 153260 | 160690 | 16957 | 132630 | 55248 | 14501 | 5438 | 3003 | 0.0135 |
| Ab101-1 | 156130 | 118080 | 78690 | 8441 | 3673 | 2774 | 2742 | 3061 | 0.2518 |

Blinatumomab and Ab101-1 were able to induce reporter gene luminescence Signals in the assay using Raji cells as target cells, the EC50 value were 0.0135 nM and 0.2518 nM respectively. Ab101-1 induced dose-dependent T cell activation with lower potency than blinatumomab, suggesting it may retain effective T cell engagement while potentially reducing the risk of cytokine release syndrome.

### Example 13. Human PBMC B Cell Depletion and Cytokine Release Assay

The objective was to evaluate the effects of Ab101-1, blinatumomab, and teclistamab on human B cell depletion and T cell activation.

Methods: Peripheral blood mononuclear cells (PBMCs) were obtained from healthy volunteers (n=6) and patients with systemic lupus erythematosus (SLE, n=6), rheumatoid arthritis (RA, n=6), or systemic sclerosis (SS, n=6). These cells were treated with a series of serially diluted concentrations of test antibodies (Ab101-1, blinatumomab, or teclistamab) for 48 hours. Following treatment, cells were harvested and stained with a live/dead dye and specific markers for T and B cells. Subsequently, the cells were washed and prepared for flow cytometry analysis. The efficiency of B cell depletion, expressed as a killing ratio, was calculated. In addition, T cell activation status and cytokine release levels were assessed.

### Results

TABLE 18
EC₅₀ of Ab101-1, blinatumomab and teclistamab on B cell killing and T cell activation (Healthy Donors)

| EC₅₀ (pM) | Test Ab | Donor 1 | Donor 2 | Donor 3 | Donor 4 | Donor 5 | Donor 6 | Mean | SD |
|---|---|---|---|---|---|---|---|---|---|
| Viable | Ab101-1 | 0.6824 | 1.118 | 0.9832 | 0.7368 | 0.5864 | 2.209 | 1.05 | 0.6 |
| B cells | Blinatumomab | 1.509 | 10.6 | 10120 | 2.589 | 0.5667 | 5.133 | 1690.07 | 4129.81 |
| | Teclistamab | 635.1 | 612 | 2163 | 425 | 672 | 19300 | 3967.85 | 7538.11 |
| Live | Ab101-1 | 0.5137 | 0.2255 | 0.5011 | 0.2072 | 0.1966 | 0.6172 | 0.38 | 0.19 |
| CD69⁺CD4⁺ | Blinatumomab | 0.8276 | 1.194 | 1.725 | 0.4055 | 0.136 | 2.811 | 1.18 | 0.98 |
| T cells | Teclistamab | 1043 | 2222 | 6432 | 920.1 | 1213 | 1888 | 2286.35 | 2093.51 |
| Live | Ab101-1 | 0.5896 | 0.2791 | 0.3226 | 0.3179 | 0.3667 | 0.5759 | 0.41 | 0.14 |
| CD69⁺CD8⁺ | Blinatumomab | 0.08098 | 0.5797 | 0.9322 | 0.2675 | 0.2469 | 2.996 | 0.85 | 1.09 |
| T cells | Teclistamab | 2725 | 6250 | 9612 | 1715 | 3455 | 5485 | 4873.67 | 2874.94 |

TABLE 19
EC₅₀ of Ab101-1, blinatumomab and teclistamab on cytokines release (Healthy Donors)

| EC₅₀ | | | | | | | | | |
|---|---|---|---|---|---|---|---|---|---|
| (pM) | Test Ab | Donor 1 | Donor 2 | Donor 3 | Donor 4 | Donor 5 | Donor 6 | Mean | SD |
| IL-2 | Ab101-1 | 423.9 | 0.2442 | 1.287 | 0.2927 | 0.4628 | 0.4632 | 71.11 | 172.83 |
| | Blinatumomab | 9.106 | 16.15 | 132.8 | 0.07876 | 1311 | 0.1155 | 244.88 | 524.77 |
| | Teclistamab | 17.82 | 589.5 | 1 | 4794 | / | / | 1800.44 | 2608.21 |
| IFN-γ | Ab101-1 | 5.442 | 5.204 | 1.133 | 3.958 | 15.85 | / | 6.32 | 5.6 |
| | Blinatumomab | / | 20.67 | / | 37.57 | 156.6 | 16.69 | 57.88 | 66.43 |
| | Teclistamab | / | 1083 | / | / | 2328 | 2578 | 1996.33 | 800.79 |
| TNF-α | Ab101-1 | 5.394 | 5.137 | 1.94 | 7.469 | 72.47 | 1.741 | 15.69 | 27.9 |
| | Blinatumomab | 14.79 | 32.36 | / | 37.76 | 296.6 | 2.625 | 76.83 | 123.65 |
| | Teclistamab | 15010 | 2681 | 2804 | 967 | 3815 | 2012 | 4548.17 | 5211 |
| IL-6 | Ab101-1 | 5.158 | 5.223 | 1.311 | 0.2269 | 120.4 | 2.407 | 22.45 | 48.03 |
| | Blinatumomab | 65.97 | 14.47 | 25.27 | 37.29 | / | 32.71 | 35.14 | 19.27 |
| | Teclistamab | 316100 | 405.6 | / | 669.9 | / | 13010 | 82546.38 | 155813.42 |

TABLE 20
EC₅₀ of Ab101-1, blinatumomab and teclistamab on B cell killing and T cell activation (RA Donors)

| EC₅₀ | | | | | | | | | |
|---|---|---|---|---|---|---|---|---|---|
| (pM) | Test Ab | Donor 1 | Donor 2 | Donor 3 | Donor 4 | Donor 5 | Donor 6 | Mean | SD |
| Viable B | Ab101-1 | 3.007 | 9.063 | 3.903 | 14.58 | 2.299 | 1.285 | 5.69 | 5.13 |
| cells | Blinatumomab | 8.265 | 24.06 | 11.08 | 256.6 | 13.98 | 3.102 | 52.85 | 100.06 |
| | Teclistamab | 2.491 | 14250 | 0.429 | / | / | 1903 | 4038.98 | 6866.11 |
| Live | Ab101-1 | 1.018 | 1.8 | 1.234 | 1.003 | 1.511 | 1.29 | 1.31 | 0.31 |
| CD69⁺CD4⁺ | Blinatumomab | 7.292 | 5.277 | 5.573 | 4.092 | 9.092 | 4.196 | 5.92 | 1.94 |
| T cells | Teclistamab | 1244 | 943.5 | 1546 | 2312 | 2140 | 1715 | 1650.08 | 521.03 |
| Live | Ab101-1 | 0.715 | 1.055 | 0.8789 | 1.439 | 1.067 | 1.115 | 1.04 | 0.24 |
| CD69⁺CD8⁺ | Blinatumomab | 2.176 | 1.453 | 0.8189 | 15.11 | 6.318 | 3.513 | 4.9 | 5.37 |
| T cells | Teclistamab | 1046 | 771.2 | 1731 | 3088 | 1996 | 2257 | 1814.87 | 841.02 |

TABLE 21
EC₅₀ of Ab101-1, blinatumomab and teclistamab on cytokines release (RA Donors)

| EC50 | | | | | | | | | |
|---|---|---|---|---|---|---|---|---|---|
| (pM) | Test Ab | Donor 1 | Donor 2 | Donor 3 | Donor 4 | Donor 5 | Donor 6 | Mean | SD |
| IL-2 | Ab101-1 | 0.4256 | 4.411 | / | 4.147 | 4.176 | 0.5323 | 2.74 | 2.07 |
| | Blinatumomab | 1.548 | 10.34 | / | 56.75 | 8.204 | 59.13 | 27.19 | 28.27 |
| | Teclistamab | 1951 | 4327 | / | 2617 | 16700 | 16630 | 8445 | 7553.72 |
| IFN-γ | Ab101-1 | 3.008 | 5.585 | 3.039 | 4.306 | 3.727 | 1.649 | 3.55 | 1.34 |
| | Blinatumomab | 15.1 | 14.97 | 8.251 | 57.66 | 12.79 | 7.137 | 19.32 | 19.08 |
| | Teclistamab | 3648 | 17140 | 2575 | 4613 | 17700 | 3767 | 8240.5 | 7142.03 |
| TNF-α | Ab101-1 | 1.765 | 8.92 | 2.97 | 3.432 | 3.586 | 1.865 | 3.76 | 2.64 |
| | Blinatumomab | 11.39 | 130.9 | 182.2 | 50.11 | 17.25 | 8.005 | 66.64 | 73.06 |
| | Teclistamab | 2385 | 21280 | 16700 | 16470 | 16600 | 21890 | 15887.5 | 7055.97 |
| IL-6 | Ab101-1 | 1.447 | 0.6238 | 983.2 | / | 5073 | 4.359 | 1212.53 | 2199.48 |
| | Blinatumomab | 7.871 | 0.8796 | / | 26.75 | 49.56 | / | 21.27 | 21.8 |
| | Teclistamab | 5.238 | 657 | 1528 | 23680 | / | / | 6467.56 | 11491.9 |

TABLE 22
EC₅₀ of Ab101-1, blinatumomab and teclistamab on B cell killing and T cell activation (SLE Donors)

| EC₅₀ | | | | | | | | | |
|---|---|---|---|---|---|---|---|---|---|
| (pM) | Test Ab | Donor 1 | Donor 2 | Donor 3 | Donor 4 | Donor 5 | Donor 6 | Mean | SD |
| Viable B | Ab101-1 | 0.1979 | 1.179 | 1.873 | 0.9827 | 0.4488 | 0.4785 | 0.86 | 0.62 |
| cells | Blinatumomab | 1585 | 1.478 | 3.961 | 5.276 | 15.34 | 0.7633 | 268.64 | 644.91 |
| | Teclistamab | 16.08 | 648.4 | 13870 | 58590 | / | 1738 | 14972.5 | 25038.61 |
| Live | Ab101-1 | 0.2843 | 1.813 | 0.9431 | 0.4944 | 3.077 | 0.1713 | 1.13 | 1.13 |
| CD69⁺CD4⁺ | Blinatumomab | 2.647 | 2.494 | 5.58 | 2.925 | 2.763 | 0.9181 | 2.89 | 1.51 |
| T cells | Teclistamab | 2023 | 23920 | 3234 | 2528 | 1944 | 421.8 | 5678.47 | 8984.38 |
| Live | Ab101-1 | 0.1423 | 0.7705 | 1.062 | 0.4106 | 0.9036 | 0.2404 | 0.59 | 0.38 |
| CD69⁺CD8⁺ | Blinatumomab | / | 1.43 | 4.522 | 1.352 | 1.265 | 0.6709 | 1.85 | 1.52 |
| T cells | Teclistamab | 1580 | 3743 | 6629 | 2944 | 1468 | 567.2 | 2821.87 | 2182.49 |

TABLE 23
EC₅₀ of Ab101-1, blinatumomab and teclistamab on cytokines release (SLE Donors)

| EC50 | | | | | | | | | |
|---|---|---|---|---|---|---|---|---|---|
| (pM) | Test Ab | Donor 1 | Donor 2 | Donor 3 | Donor 4 | Donor 5 | Donor 6 | Mean | SD |
| IL-2 | Ab101-1 | / | 1.15 | 1.464 | 0.7986 | 1.509 | 0.5138 | 1.09 | 0.43 |
| | Blinatumomab | / | 10.46 | 10.09 | 4.017 | 9.784 | 0.8136 | 7.03 | 4.37 |
| | Teclistamab | / | 969.4 | 1819 | 2075 | 1920 | 239.8 | 1404.64 | 779.9 |
| IFN-γ | Ab101-1 | 1.724 | 2.901 | 3.756 | 1.907 | 3.031 | 0.533 | 2.31 | 1.15 |
| | Blinatumomab | 2.431 | 10.68 | 20.55 | 3.762 | 8.707 | 2.357 | 8.08 | 7.01 |
| | Teclistamab | / | 5941 | 6551 | 2476 | 196.6 | 43260 | 11684.92 | 17840.41 |
| TNF-α | Ab101-1 | 1.875 | 4.457 | 3.823 | 1.844 | 3.417 | 1.194 | 2.77 | 1.31 |
| | Blinatumomab | 2.612 | 6.707 | 10.1 | 7.321 | 9.809 | 4.532 | 6.85 | 2.93 |
| | Teclistamab | / | 2460 | 26580 | 29790 | 9446 | 11010 | 15857.2 | 11759.94 |
| IL-6 | Ab101-1 | / | 135.5 | 12.71 | 33.29 | / | / | 60.5 | 65.76 |
| | Blinatumomab | 3.047 | 270200 | 5.71 | 1.477 | / | 6.068 | 54043.26 | 120835.29 |
| | Teclistamab | 134.2 | 7160 | 4389 | 21790 | / | 326900 | 72074.64 | 142684.26 |

TABLE 24
EC₅₀ of Ab101-1, blinatumomab and teclistamab on B cell killing and T cell activation (SS Donors)

| EC₅₀ | | | | | | | | | |
|---|---|---|---|---|---|---|---|---|---|
| (pM) | Test Ab | Donor 1 | Donor 2 | Donor 3 | Donor 4 | Donor 5 | Donor 6 | Mean | SD |
| Viable B | Ab101-1 | 1.368 | 1.044 | 3.573 | 4.404 | 3.187 | 3.341 | 2.82 | 1.32 |
| cells | Blinatumomab | 2.967 | 8.361 | 8.049 | 45.11 | 8.736 | 13.72 | 14.49 | 15.38 |
| | Teclistamab | / | / | / | 16200 | / | / | 16200 | / |
| Live | Ab101-1 | 1.058 | 0.9745 | 1.111 | 1.206 | 3.048 | 0.7688 | 1.36 | 0.84 |
| CD69⁺CD4⁺ | Blinatumomab | 1.295 | 1.401 | 3.153 | 8.48 | 6.628 | 4.847 | 4.3 | 2.9 |
| T cells | Teclistamab | 950.3 | 880.9 | 271.5 | 1176 | 1222 | 1011 | 918.62 | 342.93 |
| Live | Ab101-1 | 0.8352 | 1.168 | 0.6702 | 1.38 | 1.098 | 1.097 | 1.04 | 0.25 |
| CD69⁺CD8⁺ | Blinatumomab | 2.318 | 1.812 | 1.092 | 5.216 | 2.265 | 2.762 | 2.58 | 1.41 |
| T cells | Teclistamab | 3129 | 1181 | 543.2 | 1279 | 1076 | 1440 | 1441.37 | 881.19 |

TABLE 25
EC₅₀ of Ab101-1, blinatumomab and teclistamab on cytokines release (SS Donors)

| EC50 | | | | | | | | | |
|---|---|---|---|---|---|---|---|---|---|
| (pM) | Test Ab | Donor 1 | Donor 2 | Donor 3 | Donor 4 | Donor 5 | Donor 6 | Mean | SD |
| IL-2 | Ab101-1 | 1.419 | 1.771 | 0.8948 | 3.386 | 6.976 | 3.83 | 3.05 | 2.24 |
| | Blinatumomab | 5.32 | 5.144 | 8.309 | 26.14 | 18.65 | 7.815 | 11.9 | 8.57 |
| | Teclistamab | 2077 | 1877 | 2413 | 3135 | 1591 | 1784 | 2146.17 | 559.57 |
| IFN-γ | Ab101-1 | 1.786 | 2.243 | 1.438 | 2.371 | 13.08 | 3.143 | 4.01 | 4.48 |
| | Blinatumomab | 3.931 | 4.508 | 8.154 | 45.62 | 28.22 | 7.824 | 16.38 | 16.92 |
| | Teclistamab | / | 1906 | 1595 | 14870 | 3065 | 2860 | 4859.2 | 5630.46 |
| TNF-α | Ab101-1 | 2.428 | 3.096 | 2.68 | 2.621 | 6.668 | 3.687 | 3.53 | 1.6 |
| | Blinatumomab | 6.348 | 5.733 | 14.46 | 28.3 | 22.85 | 10.33 | 14.67 | 9.17 |
| | Teclistamab | 1838 | 2864 | 2405 | 3637 | 2871 | 8701 | 3719.33 | 2511.65 |
| | Ab101-1 | 5.212 | 7.947 | 1.286 | 84.68 | 17.8 | / | 23.39 | 34.8 |
| IL-6 | Blinatumomab | 7.405 | 12.42 | 787.9 | / | 26.12 | / | 208.46 | 386.37 |
| | Teclistamab | / | 2649 | / | 2574 | 17830 | 33590 | 14160.75 | 14806.89 |

Ab101-1, blinatumomab, and teclistamab each demonstrated dose-dependent efficacy in depleting human B cells. Notably, at equivalent concentrations, Ab101-1 and blinatumomab achieved greater B cell depletion than teclistamab. Additionally, T cell activation assays revealed that all three test antibodies induced concentration-dependent T cell activation.

Ab101-1, blinatumomab, and teclistamab all induced dose-dependent depletion of human B cells. Notably, at equivalent concentrations, Ab101-1 and blinatumomab exhibited greater efficacy in depleting B cells compared to teclistamab. The EC₅₀ values are summarized in Tables 18-25. Furthermore, T cell activation assays demonstrated that all three test antibodies—Ab101-1, blinatumomab, and teclistamab—induced concentration-dependent T cell activation.

### 1) T Cell Activation

Ab101-1 exhibited the highest potency across all donor groups. For CD4+ T cells, Ab101-1 achieved mean EC₅₀ values of 0.38 pM in healthy donors, 1.31 pM in RA donors, 1.13 pM in SLE donors, and 1.36 pM in SS donors. Blinatumomab showed lower potency, with corresponding EC₅₀ values of 1.18 pM, 5.92 pM, 2.89 pM, and 4.30 pM, respectively, while teclistamab was the least potent, with EC₅₀ values spanning from the picomolar to the nanomolar range. A similar trend was observed for CD8+ T cells, with Ab101-1 demonstrating EC₅₀ values of 0.41 pM in healthy donors, 1.04 pM in RA donors, 0.59 pM in SLE donors, and 1.04 pM in SS donors. Blinatumomab consistently showed higher EC₅₀ values than Ab101-1, and teclistamab again exhibited the lowest potency across all donor groups.

### 2) Cytokine Production

Cytokine release, assessed by EC₅₀ values for IL-2, IFN-γ, TNF-α, and IL-6, further underscored the differences in potency among the antibodies. Ab101-1 consistently induced cytokine production at lower concentrations compared to blinatumomab or teclistamab across all donor types. For example, in healthy donors, the EC₅₀ for IL-2 release was 71.11 pM for Ab101-1, compared with 244.88 pM for blinatumomab and 1800.44 pM for teclistamab. Similar trends were observed for IFN-γ, TNF-α, and IL-6. The same rank order of potency Ab101-1>blinatumomab>teclistamab—was generally maintained across RA, SLE, and SS donor groups. Considerable inter-donor variability was observed, particularly for blinatumomab and teclistamab, as indicated by high standard deviations.

### 3) B Cell Depletion

B cell-killing activity was evaluated across antibodies and donor groups, with potency quantified by EC₅₀ values. Ab101-1 demonstrated the highest potency in all groups, with mean EC₅₀ values of 1.05 pM in healthy donors, 5.69 pM in RA donors, 0.86 pM in SLE donors, and 2.82 pM in SS donors. These values were significantly lower than those of blinatumomab, which ranged from 14.49 pM to 1690.07 pM, and of teclistamab, which ranged from 3967.85 pM to 16200 pM. The potency advantage of Ab101-1 over blinatumomab was most evident in healthy and SLE donors, while in SS donors the difference was smaller but still statistically significant. Across all donor types, Ab101-1 consistently demonstrated superior performance in B cell depletion compared to both comparator antibodies.

### Example 14. BCMA Cell Binding and Cytotoxicity Assays of Ab101-1

The objective of this study was to evaluate the binding activity and cytotoxic potency of Ab101-1 in BCMA positive cells. A bispecific antibody with the same anti-CD19 and anti-CD3 modules as Ab101-1 but lacking the BCMA binding domain was used as a control (anti-CD3×CD19 construct). Binding to target-expressing cells was assessed by flow cytometry using CHO-S-BCMA cells, and cytotoxicity was determined in luciferase-based killing assays using T cells.

Table 26 shows the MFI of cell binding and the IC50 values from the cytotoxicity assay.

TABLE 26
Summary of Cell Binding and Cytotoxicity Results

| | | | anti-CD3 × | |
|---|---|---|---|---|
| | | | CD19 | |
| | | Ab101-1 | construct | NC |
| Assay | Cell Type | Result | Result | Result |
| Binding | CHO-S | 182 | 224 | 144 |
| (FACs MFI) | CHO-S-BCMA | 2981 | 278 | 172 |
| Cytotoxicity | CHO-BCMA- | IC₅₀ = | | |
| (Luciferase, | Luciferase | 25.6 nM | | |
| IC₅₀) | MM1.S- | IC₅₀ = | — | — |
| | Luciferase | 1.85 nM | | |

NC = no-treatment control

Ab101-1 demonstrated specific binding to BCMA-expressing cells and robust T cell-mediated cytotoxicity against BCMA-positive target cells, MM1 and CHO-BCMA in vitro, with IC₅₀values in the low nanomolar range. The anti-CD3×CD19 construct did not bind to BCMA-expressing cells. These findings supported the proposed mechanism of action of Ab101-1 as a BCMA targeting T cell engager and provided justification for further preclinical development.

### Example 15. Cytotoxicity and Cytokine Release

The aim of this example was to assess B cell killing, T cell activation, and cytokine release in fresh PBMCs treated with Ab101-1.

Fresh human PBMCs were isolated from healthy human donors and processed using ficoll density centrifugation and sodium citrate as anti-coagulant. Cell pellets were centrifuged at 500×g for 6 min at RT and re-suspended with complete medium (RPMI 1640 medium+10% v/v FBS+1% v/v Pen-Strep+1×MEM NEAA) at a density of 1.67×106 cells/mL. Test antibodies were prepared at 2.5× final concentration and serially diluted in complete media (starting at 500 nM, 6-fold, 10 point) before mixing with PBMCs. For no antibody treatment wells, complete media was added to PBMCs. 250 μL of cell—antibody mixture was then aliquoted into 4 culture wells in separate culture plates. Final cell count in each well was 2.5×105. Following incubation at 37 °C, 5% CO2 for 24 hours, plates were centrifuged at 800×g for 3 min. 200 μL of supernatant was collected and stored at −80 °C. Cell pellets from the same antibody treatment wells were washed twice with 1×DPBS and pooled for flow cytometry staining.

### Flow Cytometry Analysis

Cells were stained with 100 μL fixable viability dye eFluor780 for 20 min at RT in 1×DPBS. Following staining, cells were washed twice with stain buffer (FBS) and centrifuged at 800×g for 3 mins. 20 μL Fc Receptor Blocking Solution diluted in stain buffer (FBS) was used to resuspend cells followed by incubation at 4 °C for 15 min. For cell staining, fluorochrome conjugated antibodies and Brilliant Stain Buffer Plus were diluted in stain buffer (FBS) and 30 μL of the mixture was used to resuspend cells. Following incubation at 4 °C for 30 min, cells were washed twice and resuspended in 120 μL final volume containing 20 μL count beads. Acquisition of data was performed on a Cytek aurora flow cytometer and analyzed using flowjo software v10.0. Absolute cell counts were calculated using (Cell count×eBead volume)/(eBead count×Cell volume)×eBead concentration.

Cytokine release measurement Cytokine measurements were performed using V-PLEX Proinflammatory Panel 1 Human Kit (MSD #K15049G) according to manufacturer's instructions. Cell culture supernatants were thawed to RT and diluted 2-4× using assay media before measurements. MSD calibrators were included in each measurement plate. Cytokine concentrations were derived by backfitting electrochemiluminesence (ECL) signals against corresponding calibrator values.

Data analysis Raw data were analyzed using Microsoft excel or python language using NumPy and Pandas libraries. Graphs were generated using Graphpad prism v10 or matplotlib library. Graph fittings were performed using SciPy's function scipy.optimize.curve_fit and fitted against a four-parameter non-linear regression model, where Y=Bottom+(X\hat{ }Hillslope)*(Top−Bottom)/(X\hat{ }HillSlope+EC50\hat{ }HillSlope). E/IC50 and E/IC90 values were determined using the SciPy's function scipy.optimize.fsolve.

### Results

### 1) B Cell Depletion Potency and Efficacy

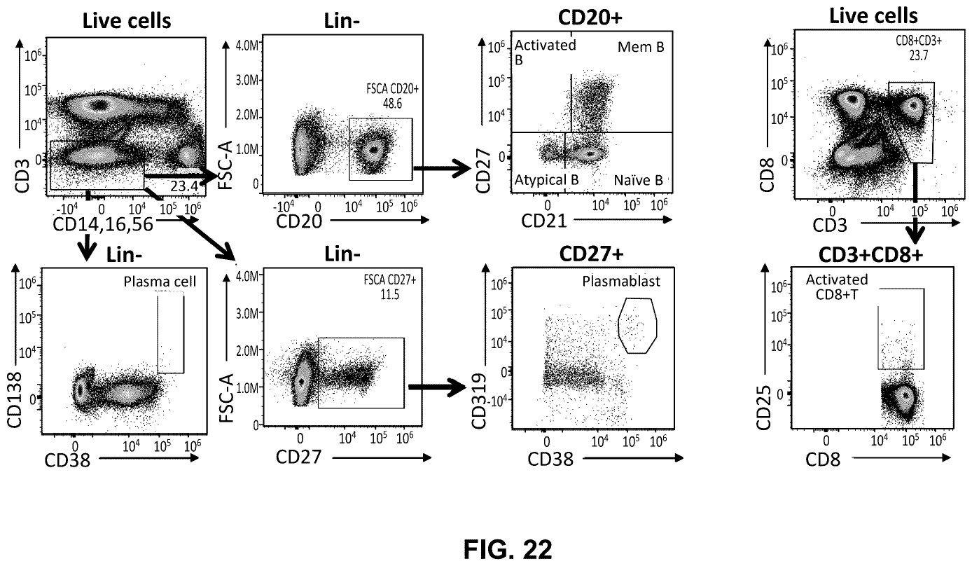

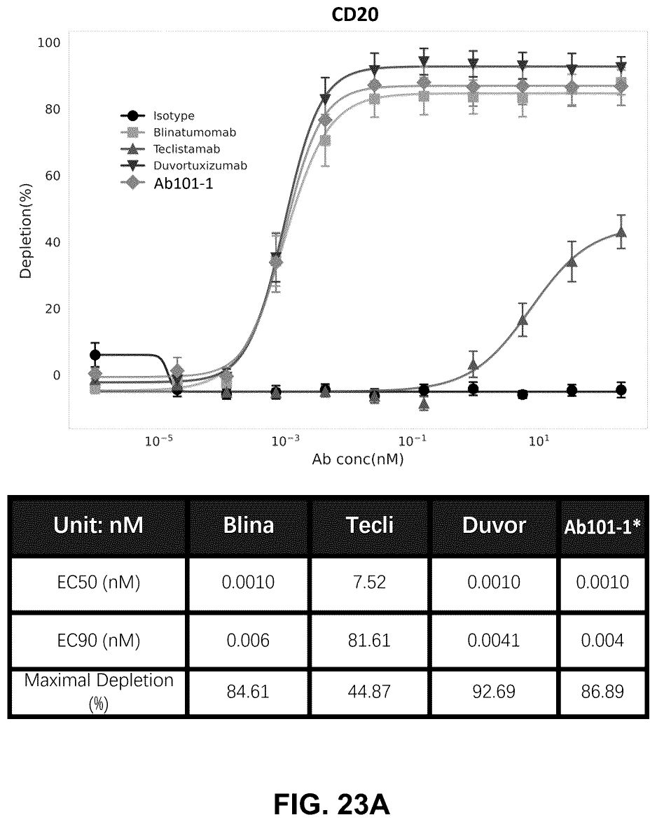

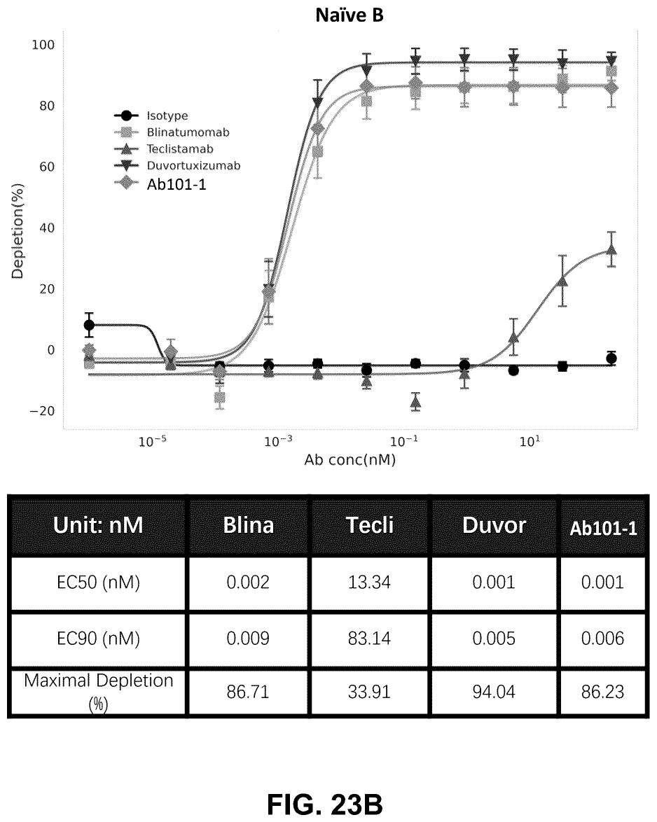

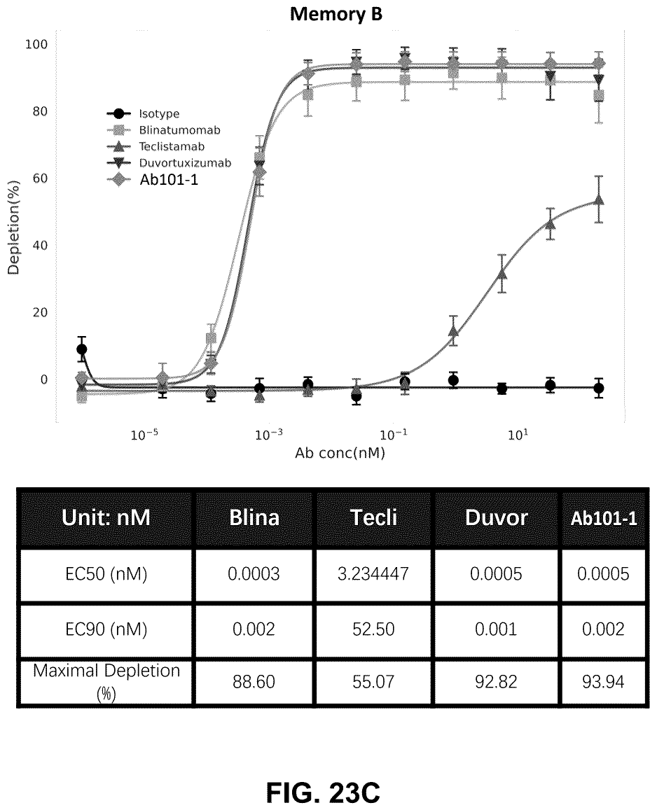

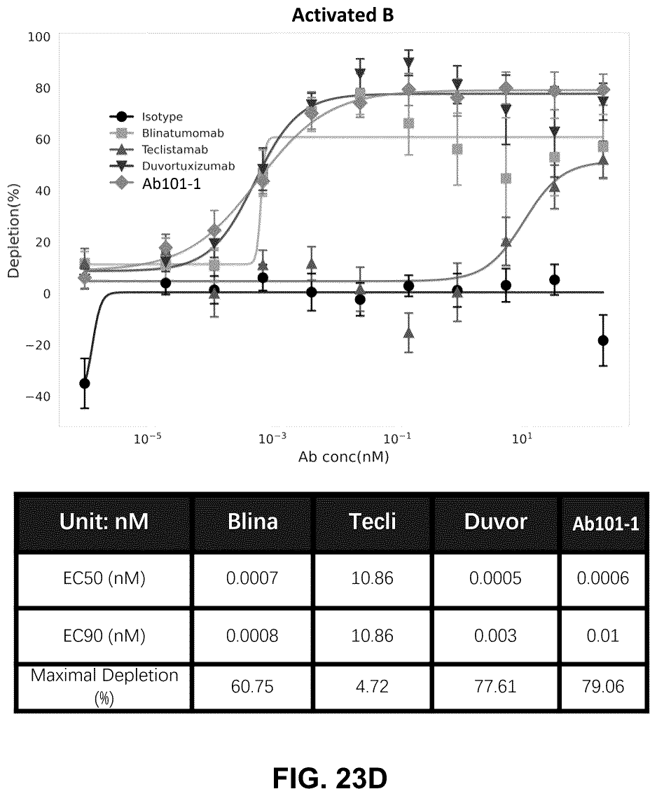

To analyze the extent of B cell depletion, B cell subsets were gated using a strategy illustrated in FIG. 22 and quantitated. FIGS. 23A-D represent the numbers of each B cell subset averaged from 10 donor PBMCs.

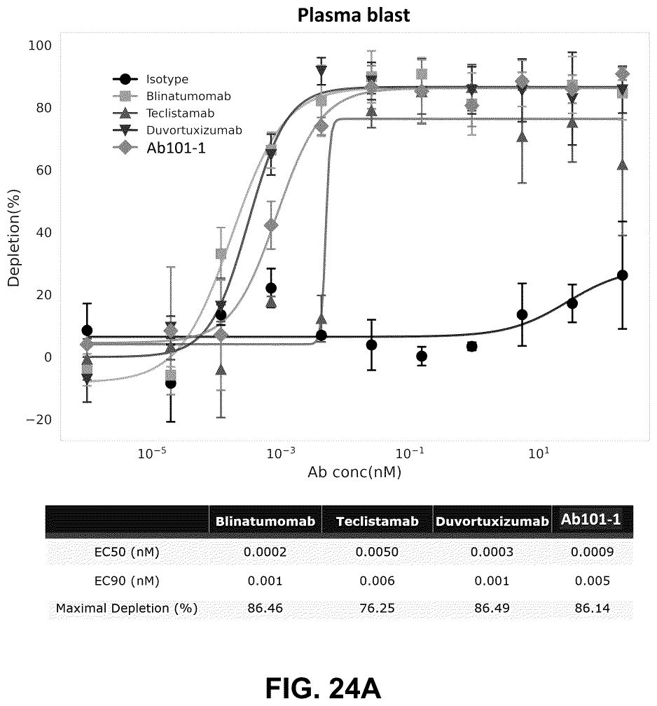

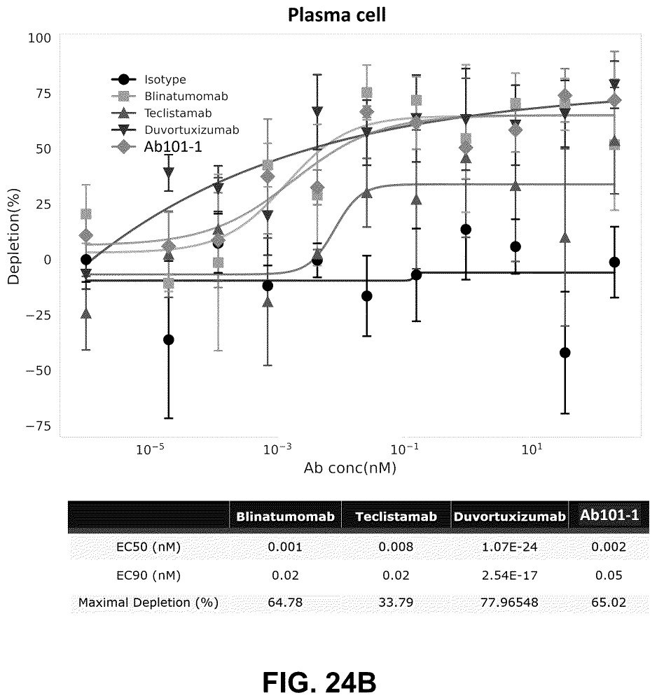

Plasmablast and plasma cell were limited in numbers in most donor PBMCs. To assess the depletion of these subsets in more detail, data was re-analyzed using PBMC donors 5, 6 and 10 (FIGS. 24A-B), which showed relatively higher initial numbers of these cell types.

### 2) T Cell Activation

TCEs exhibit functions mainly through the recruitment and activation of T cells.

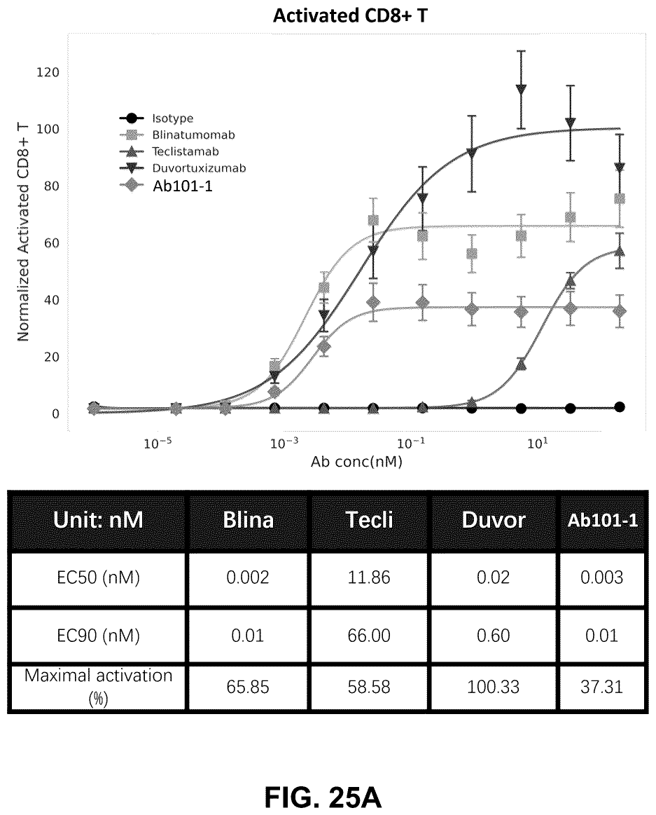

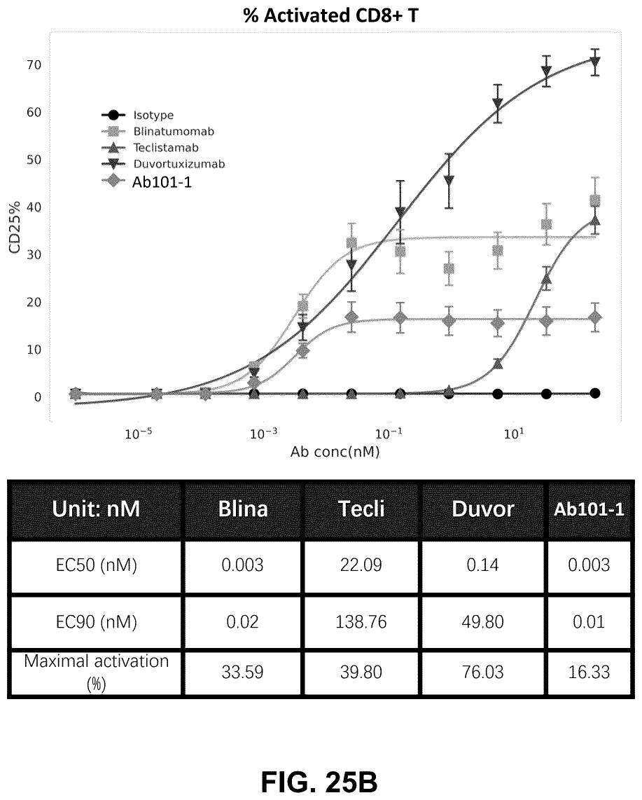

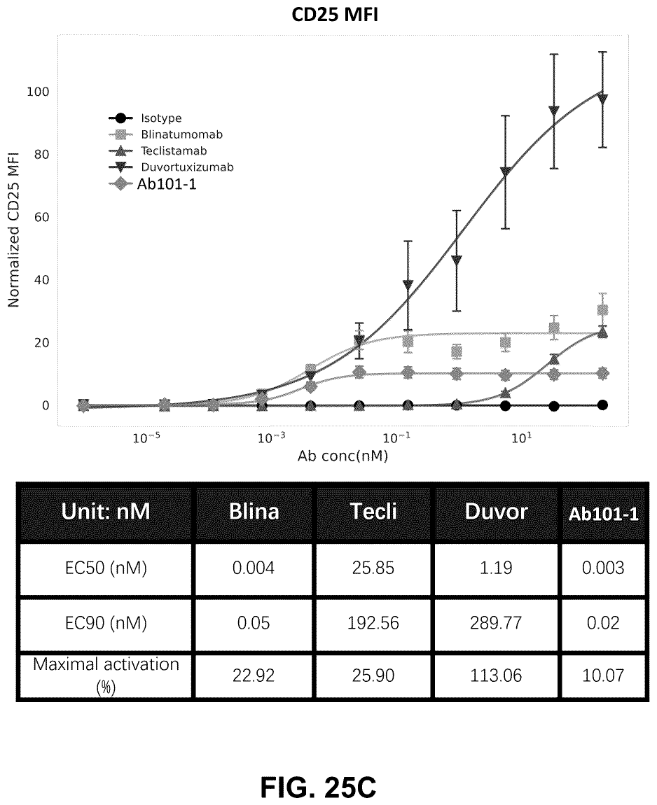

However, excessive recruitment or overactivation of T cells is also a common cause of TCE related toxicities. To assess the mechanism and relative T cell status following TCE treatment, activated CD8+ T cells were gated by flow cytometry (FIG. 22) and were analyzed for cell count (FIG. 25A), frequency (FIG. 25B), and expression of the CD25 activation marker (FIG. 25C).

### 3) Cytokine Release

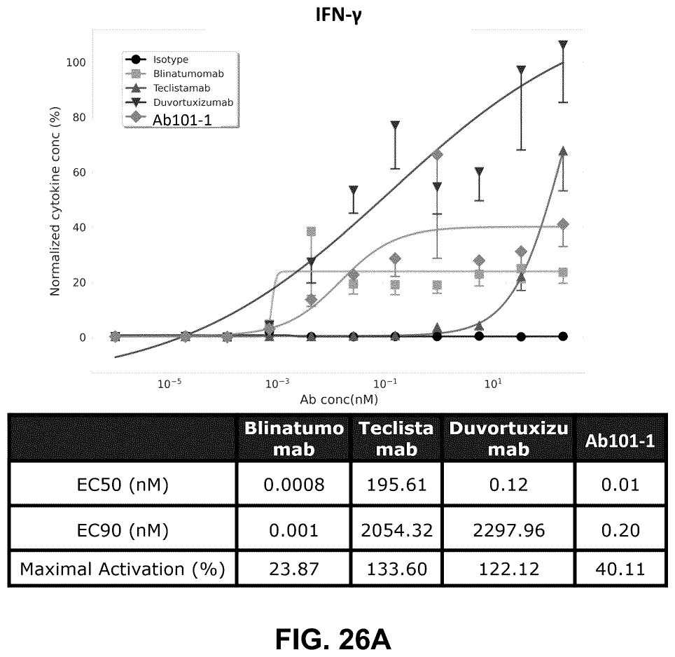

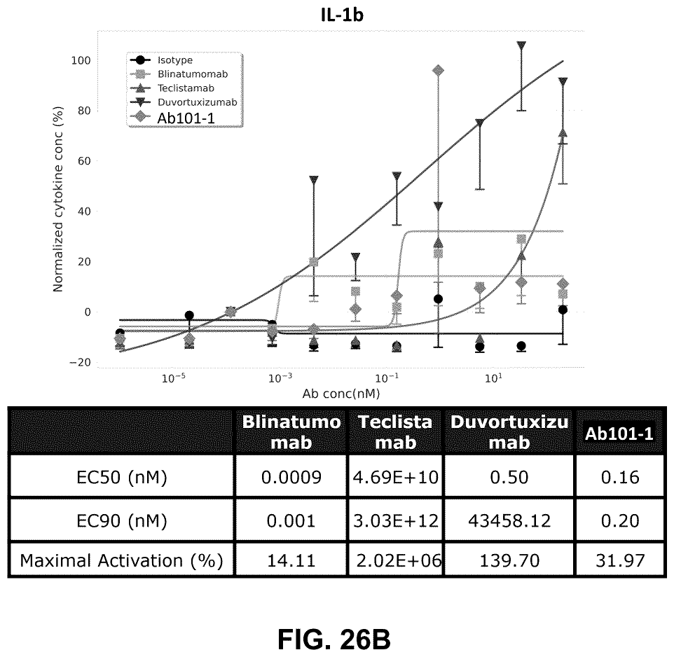

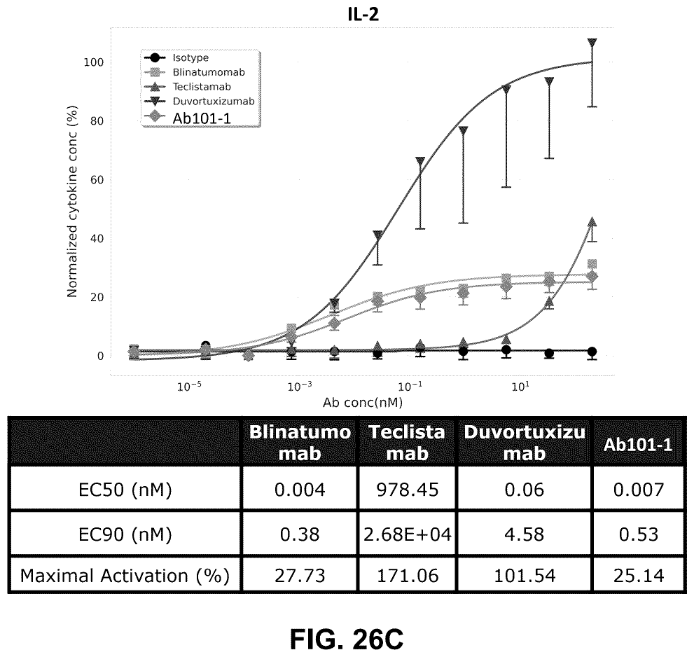

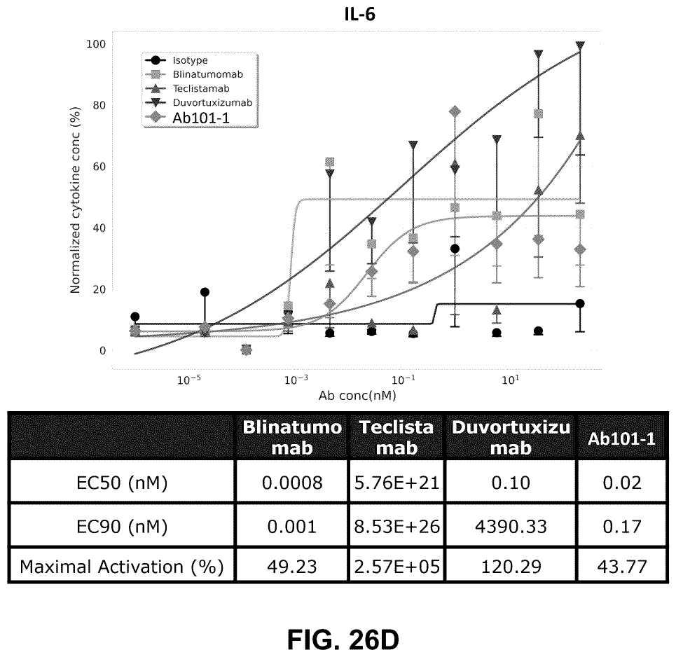

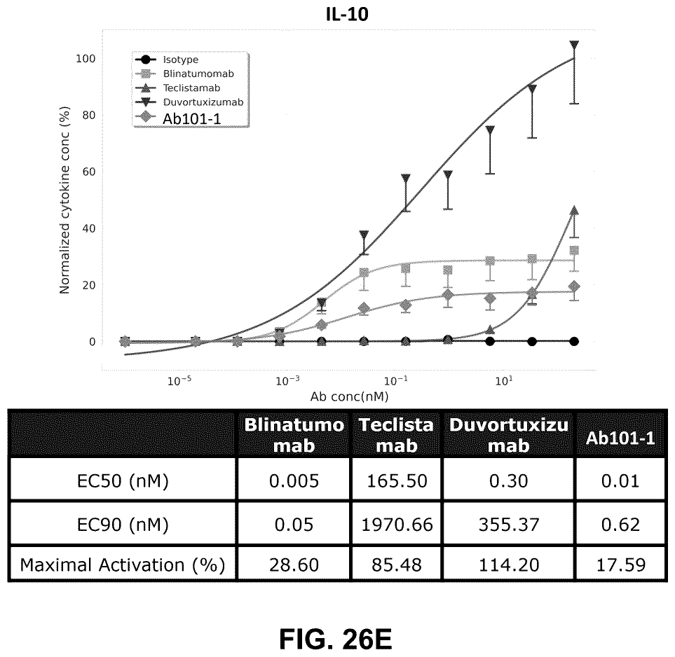

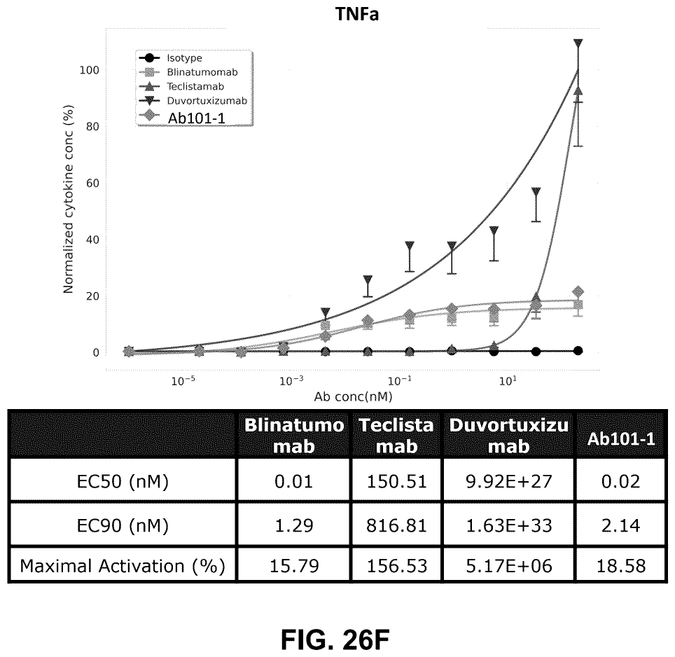

IFN-g, TNFα and IL-2 are cytokines closely associated with T cell activation and TCE responses. FIGS. 26A-F show cytokine concentrations following antibody treatment, averaged from 10 donor PBMCs.

### Example 16. 22-Day Repeated Subcutaneous Dose Range Finding Toxicity Study in Cynomolgus Monkeys

This study was carried out to determine the toxicity and toxicokinetics (TK) of Ab101-1 when administered once weekly for 4 weeks (Day 1, 8, 15 and 22, in total 4 doses) by subcutaneous injection to the cynomolgus monkey for 22 days during Phase I. In Phase II, monkeys were dosed subcutaneously once weekly for 4 weeks (Day 1, 8, 15 and 22, in total 4 doses) for 22 days to determine the toxicity and toxicokinetics of Ab101-1, but with a 5-week recovery period to evaluate the reversibility, persistence, or delayed occurrence of toxic effects. In addition, the toxicokinetics, immunogenicity and immunotoxicity of Ab101-1 were determined in all animals.

In all Ab101-1 treated animals, Ab101-1-related changes included (a) B cell depletion achieved at the lowest dose tested (0.01→0.1 mg/kg/dose), with substantial decreases in CD20+ B cells and corresponding reductions confirmed across multiple lymphoid tissues; decreased thymus weights; decreased lymphocyte cellularity in multiple lymphoid tissues; mixed cell infiltration or acute inflammation of the subcutis at injection sites; decreased immunoglobulin levels; decreased lymphocyte, leukocyte, neutrophils, monocytes, eosinophils, and basophils counts; (b) the compound was well-tolerated with no clinical signs or symptoms observed; mildly increased fibrinogen, total bilirubin, and urea; (c) low cytokine release (IL-2, IL-6, TNF-α, IFN-γ) detected only post first dose, with all levels returning to baseline within 24 hours; all changes were fully or partially reversible, except decreases in lymphocyte counts and serum immunoglobulin levels (IgG, IgM and IgA), as well as decreased lymphocyte cellularity in several lymphoid tissues. (d) No drug-related adverse events were observed. Those findings were considered not adverse based on the finding features, magnitude, and clinical well-being of the animals. Most findings observed were considered related to the pharmacological effect of Ab101-1.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

What is claimed is:

1. A multispecific antibody comprising:
(a) a first chain comprising an anti-BCMA single domain antibody (VHH), an anti-CD3 heavy chain variable region (VH), a CH1, a CH2, and a CH3;
(b) a second chain comprising a first anti-CD19 VHH, an anti-CD3 light chain variable region (VL), and a light chain constant domain (CL); and
(c) a third chain comprising a second anti-CD19 VHH, a CH2, and a CH3,
wherein
(i) the anti-BCMA VHH comprises a CDR1, CDR2, and CDR3 according, respectively, to the amino acid sequences of SEQ ID NOs:8, 9, and 10;
(ii) the first and second anti-CD19 VHH each comprise a CDR1, CDR2, and CDR3 according, respectively, to the amino acid sequences of SEQ ID NOs:3, 4, and 5;
(iii) the anti-CD3 VH comprises a H-CDR1, H-CDR2, and H-CDR3, the anti-CD3 VL comprises a L-CDR1, L-CDR2, and L-CDR3, and the H-CDR1, H-CDR2, H-CDR3, L-CDR1, L-CDR2, and L-CDR3 according, respectively, to the amino acid sequences of SEQ ID NOs:16, 17, 18, 22, 23, and 24.

2. The multispecific antibody of claim 1, wherein the anti-BCMA VHH is N-terminal to the anti-CD3 VH, the first anti-CD19 VHH is N-terminal to the anti-CD3 VL, and the second anti-CD19 VHH is N-terminal to the CH2 of the third chain.

3. The multispecific antibody of claim 1, wherein the anti-BCMA VHH comprises an amino acid sequence having at least 95% sequence identity to SEQ ID NO:6 or 7.

4. The multispecific antibody of claim 1, wherein the anti-BCMA VHH comprises the amino acid sequence of SEQ ID NO:6 or 7.

5. The multispecific antibody of claim 1, further comprising a first linker between the anti-BCMA VHH and the anti-CD3 VH.

6. The multispecific antibody of claim 1, wherein the first linker comprises the amino acid sequence of SEQ ID NO:83 or 84.

7. The multispecific antibody of claim 1, wherein the first anti-CD19 VHH and the second anti-CD19 VHH each, independently, comprises an amino acid sequence having at least 95% sequence identity to SEQ ID NO:1 or 2.

8. The multispecific antibody of claim 1, wherein the first anti-CD19 VHH and the second anti-CD19 VHH each, independently, comprises the amino acid sequence of SEQ ID NO:1 or 2.

9. The multispecific antibody of claim 1, further comprising a second linker between the first anti-CD19 VHH and the anti-CD3 VL.

10. The multispecific antibody of claim 7, wherein the second linker comprises the amino acid sequence of SEQ ID NO:83 or 84.

11. The multispecific antibody of claim 1, wherein
(a) the anti-CD3 VH comprises an amino acid sequence having at least 95% sequence identity to SEQ ID NO:15, and
(b) the anti-CD3 VL comprises an amino acid sequence having at least 95% sequence identity to SEQ ID NO:19, 20, or 21.

12. The multispecific antibody of claim 1, wherein
(a) the anti-CD3 VH comprises the amino acid sequence of SEQ ID NO:15, and
(b) the anti-CD3 VL comprises the amino acid sequence of SEQ ID NO:19, 20, or 21.

13. The multispecific antibody of claim 1, wherein
(a) wherein the anti-BCMA VHH comprises the amino acid sequence of SEQ ID NO:6 or 7,
(b) wherein the first anti-CD19 VHH and the second anti-CD19 VHH each, independently, comprises the amino acid sequence of SEQ ID NO:1 or 2,
(c) the anti-CD3 VH comprises the amino acid sequence of SEQ ID NO:15, and
(d) the anti-CD3 VL comprises the amino acid sequence of SEQ ID NO:19, 20, or 21.

14. The multispecific antibody of claim 1, wherein
(a) wherein the anti-BCMA VHH comprises the amino acid sequence of SEQ ID NO:6,
(b) wherein the first anti-CD19 VHH and the second anti-CD19 VHH each comprise the amino acid sequence of SEQ ID NO:2,
(c) the anti-CD3 VH comprises the amino acid sequence of SEQ ID NO:15, and
(d) the anti-CD3 VL comprises the amino acid sequence of SEQ ID NO:21.

15. The multispecific antibody of claim 1, wherein the CH1, CH2, and CH3 of the first chain, collectively, comprise the amino acid sequence of SEQ ID NO:57.

16. The multispecific antibody of claim 1, wherein the CL comprises the amino acid sequence of SEQ ID NO:71 or 72.

17. The multispecific antibody of claim 1, wherein the CH2 and CH3 of the third chain, collectively, comprise the amino acid sequence of SEQ ID NO:58.

18. The multispecific antibody of claim 1, wherein the first chain comprises the amino acid sequence of SEQ ID NO:118 or an amino acid sequence having at least 95% sequence identity to SEQ ID NO:118.

19. The multispecific antibody of claim 1, wherein the second chain comprises the amino acid sequence of SEQ ID NO:119 or an amino acid sequence having at least 9095% sequence identity to SEQ ID NO:119.

20. The multispecific antibody of claim 1, wherein the third chain comprises the amino acid sequence of SEQ ID NO:117 or an amino acid sequence having at least 95% sequence identity to SEQ ID NO:117.

21. The multispecific antibody of claim 1, wherein the first chain, the second chain, and the third chain comprise, respectively, the amino acid sequences of SEQ ID NOs:118, 119, and 117.

22. The multispecific antibody of claim 1, wherein the first chain, the second chain, and the third chain comprise, respectively, the amino acid sequences of SEQ ID NOs:123, 119, and 122.

23. A multispecific antibody comprising:
(a) a first chain comprising an anti-BCMA single domain antibody (VHH), an anti-CD3 heavy chain variable region (VH), a CH1, a CH2, and a CH3;
(b) a second chain comprising a first anti-CD19 VHH, an anti-CD3 light chain variable region (VL), and a light chain constant domain (CL); and
(c) a third chain comprising a second anti-CD19 VHH, a CH2, and a CH3.

24. A multispecific antibody comprising:
(a) an anti-BCMA VHH, comprising a CDR1, CDR2, and CDR3 according, respectively, to the amino acid sequences of SEQ ID NOs:8, 9, and 10;
(b) at least one anti-CD19 VHH, comprising a CDR1, CDR2, and CDR3 according, respectively, to the amino acid sequences of SEQ ID NOs:3, 4, and 5;
(c) an anti-CD3 antibody or antigen-binding fragment thereof, comprising a VH and a VL, wherein the VH comprises a H-CDR1, H-CDR2, and H-CDR3, and the VL comprises a L-CDR1, L-CDR2, and L-CDR3, and the H-CDR1, H-CDR2, H-CDR3, L-CDR1, L-CDR2, and L-CDR3 correspond, respectively, to the amino acid sequences of SEQ ID NOs:16, 17, 18, 22, 23, and 24.

25. A composition comprising the multispecific antibody of claim 1, and a pharmaceutically acceptable carrier.

26. One or more polynucleotides encoding the multispecific antibody of claim 1.

27. An isolated cell comprising the one or more polynucleotides of claim 21.

28. A method of treating an autoimmune disease or cancer in a patient in need thereof, comprising administering to the patient the multispecific antibody of claim 1.


SEQUENCE LISTING (123 sequences)

SEQ ID NO: 1 - protein, 125 aa, synthetic construct
QVQLVESGGGLVQDGDSLWLSCAASGAFFGVGIGWFRQAPGKEREFVAAITWSGGRTWYTKDVKGRFIISADKPRNTVLLQMNDLKSEDTAVYYCASGDPSGGAGPESVRQYLHWGQGTQVTVSS

SEQ ID NO: 2 - protein, 125 aa, synthetic construct
EVQLVESGGGLVQPGGSLRLSCAASGAFFGVGIGWFRQAPGKGREFVAAITWSGGRTWYTKDVKGRFTISADKSKNTVYLQMNSLRAEDTAVYYCASGDPSGGAGPESVRQYLHWGQGTLVTVSS

SEQ ID NO: 3 - protein, 6 aa, synthetic construct
GAFFGV

SEQ ID NO: 4 - protein, 6 aa, synthetic construct
TWSGGR

SEQ ID NO: 5 - protein, 17 aa, synthetic construct
GDPSGGAGPESVRQYLH

SEQ ID NO: 6 - protein, 120 aa, synthetic construct
EVQLVESGGGLVQPGGSLRLSCATSGFTFSPSAMNWVRQAPGKGLEWVADIYGDGRAVYADSVKGRFTVSRDNSKNLVYLQMNSLRAEDTAVYVCGVGRYTTTPLSEKLWGQGTLVTVSS

SEQ ID NO: 7 - protein, 120 aa, synthetic construct
EVQLVESGGGLVQPGGSLRLSCATSGFTFSPSAMNWVRQAPGEGLEWVADIYGDGRAVYADSVKGRFTVSRDNAKKLVYLEMNDLRPEDTAVYVCGVGRYTTTPLSEKLRGQGTLVTVSS

SEQ ID NO: 8 - protein, 7 aa, synthetic construct
GFTFSPS

SEQ ID NO: 9 - protein, 5 aa, synthetic construct
YGDGR

SEQ ID NO: 10 - protein, 12 aa, synthetic construct
GRYTTTPLSEKL

SEQ ID NO: 11 - protein, 122 aa, synthetic construct
QVQLVESGGGLVQPGGSLRLTCVASGSGDNFFSMGWFRQAPGKQREWVAFIYDQRATLYRDSVKGRFTISRDSAKNTAFLQMNSLKPEDTGVYYCNVILDSGTGDSDRSTYWGQGTQVTVSS

SEQ ID NO: 12 - protein, 7 aa, synthetic construct
GSGDNFF

SEQ ID NO: 13 - protein, 5 aa, synthetic construct
YDQRA

SEQ ID NO: 14 - protein, 14 aa, synthetic construct
ILDSGTGDSDRSTY

SEQ ID NO: 15 - protein, 125 aa, synthetic construct
EVQLVESGGGLVQPGGSLKLSCAASGFTFNTYAMNWVRQASGKGLEWVARIRSKYNNYATYYADSVKDRFTISRDDSLNTLYLQMNSLKTEDTAVYYCVRHGNFGNSYVSWFAYWGQGTLVTVSS

SEQ ID NO: 16 - protein, 5 aa, synthetic construct
TYAMN

SEQ ID NO: 17 - protein, 19 aa, synthetic construct
RIRSKYNNYATYYADSVKD

SEQ ID NO: 18 - protein, 14 aa, synthetic construct
HGNFGNSYVSWFAY

SEQ ID NO: 19 - protein, 109 aa, synthetic construct
QAVVTQEPSLTVSPGGTVTLTCRSSTGAVTTSNYANWVQEKPGQLFRGLIGGTNKRAPWVPARFSGSLLGDKAALTLSGAQPEDEAEYFCALWYSNLWVFGGGTKLTVL

SEQ ID NO: 20 - protein, 109 aa, synthetic construct
QAVVTQEPSLTVSPGGTVTLTCRSSTGAVTTSNYANWVQQKPGQAFRGLIGGTNKRAPWTPARFSGSLLGGKAALTLSGAQPEDEAEYFCALWYSNLWVFGGGTKLTVL

SEQ ID NO: 21 - protein, 109 aa, synthetic construct
QTVVTQEPSLTVSPGGTVTLTCRSSTGAVTTSNYANWVQEKPGQLFRGLIGGTNKRAPWVPARFSGSLLGDKAALTLSGAQPEDEAEYFCALWYSNLWVFGGGTKLTVL

SEQ ID NO: 22 - protein, 14 aa, synthetic construct
RSSTGAVTTSNYAN

SEQ ID NO: 23 - protein, 7 aa, synthetic construct
GTNKRAP

SEQ ID NO: 24 - protein, 9 aa, synthetic construct
ALWYSNLWV

SEQ ID NO: 25 - protein, 125 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 26 - protein, 109 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 27-50 - skipped (24 entries)

SEQ ID NO: 51 - protein, 226 aa, synthetic construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 230 aa, synthetic construct
EPSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 55 - protein, 226 aa, synthetic construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 56 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 57 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 58 - protein, 231 aa, synthetic construct
EPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 59-70 - skipped (12 entries)

SEQ ID NO: 71 - protein, 107 aa, synthetic construct [sequence omitted]

SEQ ID NO: 72 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 73-80 - skipped (8 entries)

SEQ ID NO: 81 - protein, 7 aa, synthetic construct
GSGGGGS

SEQ ID NO: 82 - protein, 7 aa, synthetic construct
GGGGSGS

SEQ ID NO: 83 - protein, 12 aa, synthetic construct
GSGGGGSGGGGS

SEQ ID NO: 84 - protein, 12 aa, synthetic construct
GGGGSGGGGSGS

SEQ ID NO: 85 - protein, 20 aa, synthetic construct
GGGGSGGGGSGGGGSGGGGS

SEQ ID NO: 86 - protein, 22 aa, synthetic construct
GSGGGGSGGGGSGGGGSGGGGS

SEQ ID NO: 87 - protein, 22 aa, synthetic construct
GGGGSGGGGSGGGGSGGGGSGS

SEQ ID NO: 88-100 - skipped (13 entries)

SEQ ID NO: 101 - protein, 454 aa, synthetic construct
EVQLVESGGGLVQPGGSLKLSCAASGFTFNTYAMNWVRQASGKGLEWVARIRSKYNNYATYYADSVKDRFTISRDDSLNTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 102 - protein, 216 aa, synthetic construct
QAVVTQEPSLTVSPGGTVTLTCRSSTGAVTTSNYANWVQEKPGQLFRGLIGGTNKRAPWVPARFSGSLLGDKAALTLSGAQPEDEAEYFCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 351 aa, synthetic construct
QVQLVESGGGLVQDGDSLWLSCAASGAFFGVGIGWFRQAPGKEREFVAAITWSGGRTWYTKDVKGRFIISADKPRNTVLLQMNDLKSEDTAVYYCASGDPSGGAGPESVRQYLHWGQGTQVTVSSDKTHTCPPCPAPEAAGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 454 aa, synthetic construct
EVQLVESGGGLVQPGGSLKLSCAASGFTFNTYAMNWVRQASGKGLEWVARIRSKYNNYATYYADSVKDRFTISRDDSLNTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 105 - protein, 216 aa, synthetic construct
QAVVTQEPSLTVSPGGTVTLTCRSSTGAVTTSNYANWVQQKPGQAFRGLIGGTNKRAPWTPARFSGSLLGGKAALTLSGAQPEDEAEYFCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 496 aa, synthetic construct
EVQLVESGGGLVQPGGSLRLSCAASGAFFGVGIGWFRQAPGKGREFVAAITWSGGRTWYTKDVKGRFTISADKSKNTVYLQMNSLRAEDTAVYYCASGDPSGGAGPESVRQYLHWGQGTLVTVSSGGGGSGGGGSGGGGSGGGGSEVQLVESGGGLVQPGGSLRLSCAASGAFFGVGIGWFRQAPGKGREFVAAITWSGGRTWYTKDVKGRFTISADKSKNTVYLQMNSLRAEDTAVYYCASGDPSGGAGPESVRQYLHWGQGTLVTVSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 107 - protein, 584 aa, synthetic construct
QVQLVESGGGLVQPGGSLRLTCVASGSGDNFFSMGWFRQAPGKQREWVAFIYDQRATLYRDSVKGRFTISRDSAKNTAFLQMNSLKPEDTGVYYCNVILDSGTGDSDRSTYWGQGTQVGSGGGGSGGGGSEVQLVESGGGLVQPGGSLKLSCAASGFTFNTYAMNWVRQASGKGLEWVARIRSKYNNYATYYADSVKDRFTISRDDSLNTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 108 - protein, 348 aa, synthetic construct
QVQLVESGGGLVQDGDSLWLSCAASGAFFGVGIGWFRQAPGKEREFVAAITWSGGRTWYTKDVKGRFIISADKPRNTVLLQMNDLKSEDTAVYYCASGDPSGGAGPESVRQYLHWGQGTQVTVSSGGGGSGSQAVVTQEPSLTVSPGGTVTLTCRSSTGAVTTSNYANWVQEKPGQLFRGLIGGTNKRAPWVPARFSGSLLGDKAALTLSGAQPEDEAEYFCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 230 aa, synthetic construct; identical to SEQ ID NO: 52

SEQ ID NO: 110 - protein, 591 aa, synthetic construct
EVQLVESGGGLVQPGGSLRLSCAASGAFFGVGIGWFRQAPGKGREFVAAITWSGGRTWYTKDVKGRFTISADKSKNTVYLQMNSLRAEDTAVYYCASGDPSGGAGPESVRQYLHWGQGTLVTVSSGSGGGGSGGGGSEVQLVESGGGLVQPGGSLKLSCAASGFTFNTYAMNWVRQASGKGLEWVARIRSKYNNYATYYADSVKDRFTISRDDSLNTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 111 - protein, 230 aa, synthetic construct; identical to SEQ ID NO: 52

SEQ ID NO: 112 - protein, 348 aa, synthetic construct
EVQLVESGGGLVQPGGSLRLSCATSGFTFSPSAMNWVRQAPGKGLEWVADIYGDGRAVYADSVKGRFTVSRDNSKNLVYLQMNSLRAEDTAVYVCGVGRYTTTPLSEKLWGQGTLVTVSSGGGGSGGGGSGSQAVVTQEPSLTVSPGGTVTLTCRSSTGAVTTSNYANWVQEKPGQLFRGLIGGTNKRAPWVPARFSGSLLGDKAALTLSGAQPEDEAEYFCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 348 aa, synthetic construct
EVQLVESGGGLVQPGGSLRLSCATSGFTFSPSAMNWVRQAPGEGLEWVADIYGDGRAVYADSVKGRFTVSRDNAKKLVYLEMNDLRPEDTAVYVCGVGRYTTTPLSEKLRGQGTLVTVSSGGGGSGGGGSGSQAVVTQEPSLTVSPGGTVTLTCRSSTGAVTTSNYANWVQEKPGQLFRGLIGGTNKRAPWVPARFSGSLLGDKAALTLSGAQPEDEAEYFCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 355 aa, synthetic construct
EVQLVESGGGLVQPGGSLRLSCAASGAFFGVGIGWFRQAPGKGREFVAAITWSGGRTWYTKDVKGRFTISADKSKNTVYLQMNSLRAEDTAVYYCASGDPSGGAGPESVRQYLHWGQGTLVTVSSEPSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 586 aa, synthetic construct
EVQLVESGGGLVQPGGSLRLSCATSGFTFSPSAMNWVRQAPGKGLEWVADIYGDGRAVYADSVKGRFTVSRDNSKNLVYLQMNSLRAEDTAVYVCGVGRYTTTPLSEKLWGQGTLVTVSSGSGGGGSGGGGSEVQLVESGGGLVQPGGSLKLSCAASGFTFNTYAMNWVRQASGKGLEWVARIRSKYNNYATYYADSVKDRFTISRDDSLNTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 116 - protein, 353 aa, synthetic construct
EVQLVESGGGLVQPGGSLRLSCAASGAFFGVGIGWFRQAPGKGREFVAAITWSGGRTWYTKDVKGRFTISADKSKNTVYLQMNSLRAEDTAVYYCASGDPSGGAGPESVRQYLHWGQGTLVTVSSGGGGSGGGGSGSQAVVTQEPSLTVSPGGTVTLTCRSSTGAVTTSNYANWVQEKPGQLFRGLIGGTNKRAPWVPARFSGSLLGDKAALTLSGAQPEDEAEYFCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 356 aa, synthetic construct
EVQLVESGGGLVQPGGSLRLSCAASGAFFGVGIGWFRQAPGKGREFVAAITWSGGRTWYTKDVKGRFTISADKSKNTVYLQMNSLRAEDTAVYYCASGDPSGGAGPESVRQYLHWGQGTLVTVSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - protein, 586 aa, synthetic construct; identical to SEQ ID NO: 115

SEQ ID NO: 119 - protein, 353 aa, synthetic construct
EVQLVESGGGLVQPGGSLRLSCAASGAFFGVGIGWFRQAPGKGREFVAAITWSGGRTWYTKDVKGRFTISADKSKNTVYLQMNSLRAEDTAVYYCASGDPSGGAGPESVRQYLHWGQGTLVTVSSGGGGSGGGGSGSQTVVTQEPSLTVSPGGTVTLTCRSSTGAVTTSNYANWVQEKPGQLFRGLIGGTNKRAPWVPARFSGSLLGDKAALTLSGAQPEDEAEYFCALWYSNLWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 232 aa, synthetic construct
EPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 122 - protein, 357 aa, synthetic construct
EVQLVESGGGLVQPGGSLRLSCAASGAFFGVGIGWFRQAPGKGREFVAAITWSGGRTWYTKDVKGRFTISADKSKNTVYLQMNSLRAEDTAVYYCASGDPSGGAGPESVRQYLHWGQGTLVTVSSEPKSSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 587 aa, synthetic construct
EVQLVESGGGLVQPGGSLRLSCATSGFTFSPSAMNWVRQAPGKGLEWVADIYGDGRAVYADSVKGRFTVSRDNSKNLVYLQMNSLRAEDTAVYVCGVGRYTTTPLSEKLWGQGTLVTVSSGSGGGGSGGGGSEVQLVESGGGLVQPGGSLKLSCAASGFTFNTYAMNWVRQASGKGLEWVARIRSKYNNYATYYADSVKDRFTISRDDSLNTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK